(19) United States
(12) Patent Application Publication
(10) Pub. No.: US 2024/0199576 A1
(43) Pub. Date: Jun. 20, 2024

(54) NOVEL CYCLOPENTA[C]PYRROL NEGATIVE ALLOSTERIC MODULATORS OF NR2B

(71) Applicants: Novartis AG, Basel (CH); Cadent Therapeutics, Inc., East Hanover, NJ (US)

(72) Inventors: Mark Patrick HEALY, Cambridge, MA (US); Yue PAN, Cambridge, MA (US); Kate Yaping WANG, Cambridge, MA (US)

(21) Appl. No.: 18/551,699

(22) PCT Filed: Mar. 24, 2022
(86) PCT No.: PCT/US2022/021625

§ 371 Date: Sep. 21, 2023

Related U.S. Application Data
(60) Provisional application No. 63/166,520, filed on Mar. 26, 2021.

(51) Int. Cl. C07D 401/06; A61K 31/403; A61K 31/4439; C07D 209/52; C07D 401/12

(52) U.S. Cl. CPC C07D 401/06; A61K 31/403; A61K 31/4439; C07D 209/52; C07D 401/12

(57) ABSTRACT

The present disclosure provides a compound of formula (I), or a pharmaceutically acceptable salt thereof; a method for manufacturing the compounds of the disclosure, and its therapeutic uses. The present disclosure further provides a combination of pharmacologically active agents and a pharmaceutical composition.

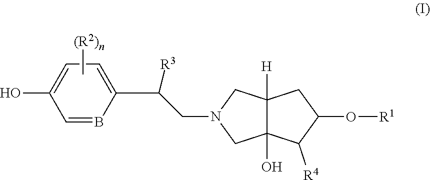

### FIELD OF THE DISCLOSURE

The present disclosure relates to compounds that selectively modulate the activity of NR1/NR2B receptors.

### BACKGROUND OF THE DISCLOSURE

The NMDA receptor is arguably an important signaling mechanism in the human brain. The brain processes a complex array of information to allow humans to function, storing information from the past and analyzing this information in the context of the present to respond and plan for the future. These incredibly complex computations are mediated at the molecular level by the continual adjustment of the strength of synapses, the nodes for communication between nerve cells (estimated at about 60 trillion in the human brain).

Glutamate is the major excitatory neurotransmitter in the brain, utilized at 80% of these synapses. NMDA receptors are one of three classes that mediate synaptic transmission using glutamate. NMDA receptors play a critical role in regulating the strength of synapses, that is, in regulating synaptic plasticity. Thus, the NMDA receptor is at the molecular core of brain function, and in particular the cognitive functions of learning and memory. These facts underlie the tremendous therapeutic utility of modulating NMDA receptor function with new drugs to treat a broad range of neuropsychiatric disease and cognitive dysfunction.

The molecular basis of NMDA receptor function is increasingly well understood. The NMDA receptor is composed of four protein subunits, two NR1 subunits and two NR2 subunits. An NR1 subunit derived from a single gene is ubiquitously expressed throughout the brain and is common to all NMDA receptors. However, the four different NR2 subunits, NR2A-D, are derived from separate genes that are differentially expressed in different brain regions and by distinct populations of neurons within a particular region. Furthermore, individual neurons may express more than one NR2 subunit and individual NMDA receptors expressed by such neurons may contain two of the same NR2 subunits (for example, 2 NR2B subunits) or two different subunits (one NR2A and one NR2B subunit). Therefore, a drug that selectively modulates the activity of one NR2 subunit may do so at receptors that express two of the targeted subunits, or only one of the targeted subunits. Thus there is a need for new treatments for diseases related to the NR1/NR2B receptor.

### SUMMARY OF THE DISCLOSURE

Various embodiments of the disclosure are described herein.

Within certain aspects, provided herein is a compound of formula (I) or a pharmaceutically acceptable salt thereof:

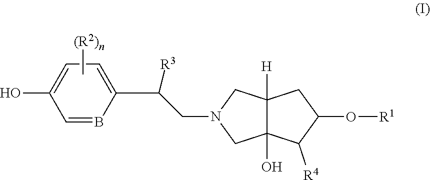

In another aspect, the disclosure provides a pharmaceutical composition comprising a compound of formula (I) or a pharmaceutically acceptable salt thereof.

The present disclosure further pertains to compounds that selectively modulate the activity of NMDA receptors that contain an NR2B subunit, which encompasses receptors containing two NR2B subunits or one NR2B subunit in combination with one other NR2 subunit (i.e., NR2A/NR2B, NR2B/NR2C, or NR2B/NR2D receptors). Such compounds can decrease the activity of NR2B-containing NMDA receptors. The present disclosure also pertains to the therapeutic uses of such compounds.

In a further aspect, the disclosure provides for a compound of formula (I), or a pharmaceutically acceptable salt thereof for use in therapy, in particular in the treatment of Parkinson's disease, Huntington's disease, Rett syndrome, amyotrophic lateral sclerosis, multiple sclerosis, seizure disorders, autism, autism spectrum disorders, Fragile X syndrome, tuberous sclerosis, Down's syndrome, pain, migraine, tinnitus, bipolar disorder, obsessive-compulsive disorder, anxiety disorder, post-traumatic stress disorder (PTSD), cocaine use disorder, major depressive disorder, refractory or treatment resistant depression, or suicidality, comprising administration of a therapeutically effective amount of a compound.

### DETAILED DESCRIPTION OF THE DISCLOSURE

The disclosure therefore provides a compound of formula (I):

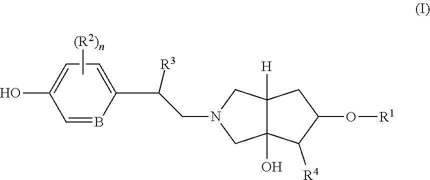

or a pharmaceutically acceptable salt thereof, wherein:
R¹ is a C₃₋₈cycloalkyl, three to seven membered heterocyclyl, phenyl, naphthyl, or heteroaryl, each of which is optionally substituted with one or more R⁵;
R² is OH, CN, halogen, OR⁶, SH, SR⁶, C₁₋₆ alkyl, haloC₁₋₆alkyl, NH₂, NHR⁶, hydroxyC₁₋₆ alkyl, N(R⁶)(R^{6′}), NHS(O)₂R⁶, or NHCOR⁶;
R³ is H, O, or OH;
R⁴ is H or OH;
R⁵ is halogen, OH, C₁₋₆alkyl, haloC₁₋₆alkyl, OR⁶, CN, NH₂, NHR⁶, N(R⁶)(R^{6′}), SH, SR⁶, SOR⁶, SO₂R⁶, SO₂NHR⁶, SO₂N(R⁶)(R^{6′}), CONH₂, CONHR⁶, or CON(R⁶)(R^{6′});
each R⁶ and R^{6′} is independently selected from the group consisting of H, O—C₁₋₆alkyl, C₁₋₆ alkyl, and haloC₁₋₆alkyl;
B is N or CRx;
each Rx is independently H, C₁₋₃ alkyl, or halogen; and
each n is independently 0, 1, or 2.

One embodiment is a compound of Formula (II):

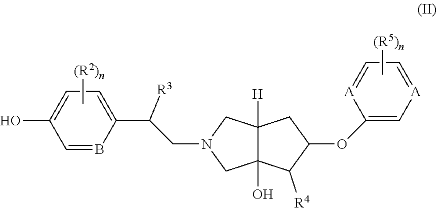

or a pharmaceutically acceptable salt thereof, wherein:
R² is OH, CN, halogen, OR⁶, SH, SR⁶, C₁₋₆ alkyl, haloC₁₋₆alkyl, NH₂, NHR⁶, hydroxyC₁₋₆ alkyl, N(R⁶)(R^{6′}), NHS(O)₂R⁶, or NHCOR⁶;
R³ is H, O, or OH;
R⁴ is H or OH;
R⁵ is halogen, OH, C₁₋₆ alkyl, haloC₁₋₆alkyl, OR⁶, CN, NH₂, NHR⁶, N(R⁶)(R^{6′}), SH, SR⁶, SOR⁶, SO₂R⁶, SO₂NHR⁶, SO₂N(R⁶)(R^{6′}), CONH₂, CONHR⁶, or CON(R⁶)(R^{6′});
each R⁶ and R^{6′} is independently selected from the group consisting of H, O—C₁₋₆alkyl, C₁₋₆ alkyl, and haloC₁₋₆alkyl;
B is N or CRx;
each Rx is independently H, C₁₋₃alkyl, or halogen; and
each n is independently 0, 1, or 2.

Another embodiment is a compound of Formula (III)

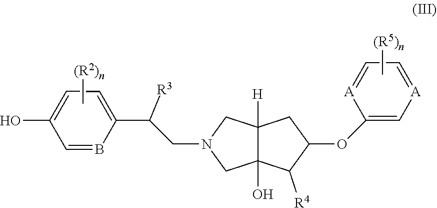

or a pharmaceutically acceptable salt thereof, wherein:
R² is halogen;
R³ is H or OH;
R⁴ is H or OH;
R⁵ is halogen;
each A is independently N or CH, provided that when one A is N the other A is CH;
B is N or CH; and
each n is independently 0, 1, or 2.

Another embodiment is a compound of Formula IIIa:

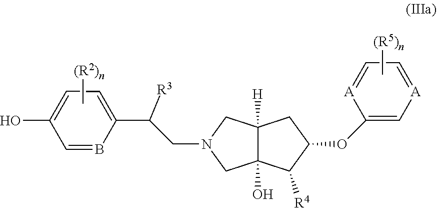

or a pharmaceutically acceptable salt thereof.

Another embodiment is a compound of Formula IIIb:

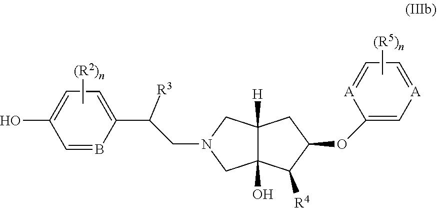

or a pharmaceutically acceptable salt thereof.

Another embodiment is a compound of Formula IIIc:

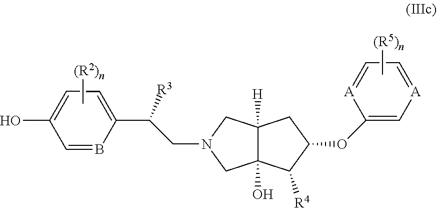

or a pharmaceutically acceptable salt thereof.

Another embodiment is a compound of Formula IIId:

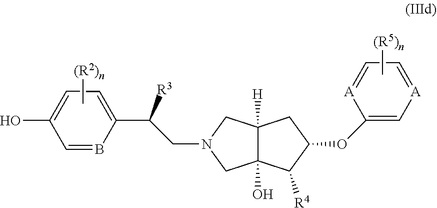

or a pharmaceutically acceptable salt thereof.

Another embodiment is a compound of Formula IIIe:

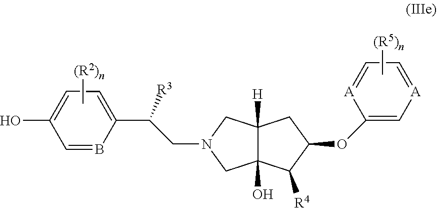

or a pharmaceutically acceptable salt thereof.

Another embodiment is a compound of Formula IIIf:

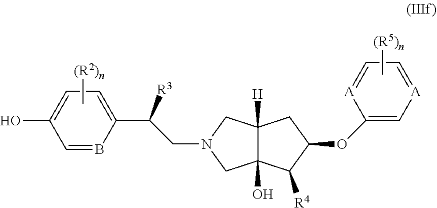

or a pharmaceutically acceptable salt thereof.

In another embodiment, R⁵ is F.

In another embodiment, R² is F or Cl.

In another embodiment, R³ is H.

In another embodiment, R³ is OH.

In another embodiment, R⁴ is H.

In another embodiment, R⁴ is OH.

In another embodiment, B is N.

In another embodiment, B is CH.

In another embodiment, A is N, provided that when one A is N the other A is CH.

In another embodiment, A is CH.

In another embodiment R² is CN, halogen, OR⁶, SH, SR⁶, C₁₋₆alkyl, haloC₁₋₆alkyl, or hydroxyC₁₋₆alkyl.

In another embodiment R² is halogen, C₁₋₆alkyl, haloC₁₋₆alkyl, or hydroxyC₁₋₆alkyl.

In another embodiment R² is halogen, C₁₋₆alkyl, or haloC₁₋₆alkyl.

In another embodiment R⁵ is halogen, OH, C₁₋₆alkyl, OR⁶, CN, SH, or SR⁶.

In another embodiment R⁵ is halogen, OH, C₁₋₆alkyl, or OR⁶.

In another embodiment R⁵ is halogen, OH, or C₁₋₆alkyl.

Specific compounds include:
(3aS,5S,6aR)-5-(2-fluorophenoxy)-2-((S)-2-hydroxy-2-(5-hydroxypyridin-2-yl)ethyl)hexahydrocyclopenta[c]pyrrol-3a(1H)-ol;
(3aS,5S,6aR)-5-(2-fluorophenoxy)-2-((R)-2-hydroxy-2-(5-hydroxypyridin-2-yl)ethyl)hexahydrocyclopenta[c]pyrrol-3a(1H)-ol;
(3aR,5R,6aS)-5-(2-fluorophenoxy)-2-((S)-2-hydroxy-2-(5-hydroxypyridin-2-yl)ethyl)hexahydrocyclopenta[c]pyrrol-3a(1H)-ol;
(3aR,5R,6aS)-5-(2-fluorophenoxy)-2-((R)-2-hydroxy-2-(5-hydroxypyridin-2-yl)ethyl)hexahydrocyclopenta[c]pyrrol-3a(1H)-ol;
(3aS,4S,5S,6aR)-5-(2-fluorophenoxy)-2-((S)-2-hydroxy-2-(5-hydroxypyridin-2-yl)ethyl)hexahydrocyclopenta[c]pyrrole-3a,4(1H)-diol;
(3aS,4S,5S,6aR)-5-(2-fluorophenoxy)-2-((R)-2-hydroxy-2-(5-hydroxypyridin-2-yl)ethyl)hexahydrocyclopenta[c]pyrrole-3a,4(1H)-diol;
(3aR,4R,5R,6aS)-5-(2-fluorophenoxy)-2-((S)-2-hydroxy-2-(5-hydroxypyridin-2-yl)ethyl)hexahydrocyclopenta[c]pyrrole-3a,4(1H)-diol;
(3aR,4R,5R,6aS)-5-(2-fluorophenoxy)-2-((R)-2-hydroxy-2-(5-hydroxypyridin-2-yl)ethyl)hexahydrocyclopenta[c]pyrrole-3a,4(1H)-diol;
(3aS,4S,5S,6aR)-2-((S)-2-(6-fluoro-5-hydroxypyridin-2-yl)-2-hydroxyethyl)-5-phenoxyhexahydrocyclopenta[c]pyrrole-3a,4(1H)-diol;
(3aS,4S,5S,6aR)-2-((R)-2-(6-fluoro-5-hydroxypyridin-2-yl)-2-hydroxyethyl)-5-phenoxyhexahydrocyclopenta[c]pyrrole-3a,4(1H)-diol;
(3aR,4R,5R,6aS)-2-((S)-2-(6-fluoro-5-hydroxypyridin-2-yl)-2-hydroxyethyl)-5-phenoxyhexahydrocyclopenta[c]pyrrole-3a,4(1H)-diol;
(3aR,4R,5R,6aS)-2-((R)-2-(6-fluoro-5-hydroxypyridin-2-yl)-2-hydroxyethyl)-5-phenoxyhexahydrocyclopenta[c]pyrrole-3a,4(1H)-diol;
(3aS,4S,5S,6aR)-2-((S)-2-(6-chloro-5-hydroxypyridin-2-yl)-2-hydroxyethyl)-5-phenoxyhexahydrocyclopenta[c]pyrrole-3a,4(1H)-diol;
(3aS,4S,5S,6aR)-2-((R)-2-(6-chloro-5-hydroxypyridin-2-yl)-2-hydroxyethyl)-5-phenoxyhexahydrocyclopenta[c]pyrrole-3a,4(1H)-diol;
(3aR,4R,5R,6aS)-2-((S)-2-(6-chloro-5-hydroxypyridin-2-yl)-2-hydroxyethyl)-5-phenoxyhexahydrocyclopenta[c]pyrrole-3a,4(1H)-diol;
(3aR,4R,5R,6aS)-2-((R)-2-(6-chloro-5-hydroxypyridin-2-yl)-2-hydroxyethyl)-5-phenoxyhexahydrocyclopenta[c]pyrrole-3a,4(1H)-diol;
(3aS,4S,5S,6aR)-2-((R)-2-(3,5-difluoro-4-hydroxyphenyl)-2-hydroxyethyl)-5-phenoxyhexahydrocyclopenta[c]pyrrole-3a,4(1H)-diol;
(3aS,4S,5S,6aR)-2-((S)-2-(3,5-difluoro-4-hydroxyphenyl)-2-hydroxyethyl)-5-phenoxyhexahydrocyclopenta[c]pyrrole-3a,4(1H)-diol;
(3aR,4R,5R,6aS)-2-((R)-2-(3,5-difluoro-4-hydroxyphenyl)-2-hydroxyethyl)-5-phenoxyhexahydrocyclopenta[c]pyrrole-3a,4(1H)-diol;
(3aR,4R,5R,6aS)-2-((S)-2-(3,5-difluoro-4-hydroxyphenyl)-2-hydroxyethyl)-5-phenoxyhexahydrocyclopenta[c]pyrrole-3a,4(1H)-diol;
(3aS,4S,5S,6aR)-2-((R)-2-(3,5-difluoro-4-hydroxyphenyl)-2-hydroxyethyl)-5-(2-fluorophenoxy)hexahydrocyclopenta[c]pyrrole-3a,4(1H)-diol;
(3aS,4S,5S,6aR)-2-((S)-2-(3,5-difluoro-4-hydroxyphenyl)-2-hydroxyethyl)-5-(2-fluorophenoxy)hexahydrocyclopenta[c]pyrrole-3a,4(1H)-diol;
(3aR,4R,5R,6aS)-2-((R)-2-(3,5-difluoro-4-hydroxyphenyl)-2-hydroxyethyl)-5-(2-fluorophenoxy)hexahydrocyclopenta[c]pyrrole-3a,4(1H)-diol;
(3aR,4R,5R,6aS)-2-((S)-2-(3,5-difluoro-4-hydroxyphenyl)-2-hydroxyethyl)-5-(2-fluorophenoxy)hexahydrocyclopenta[c]pyrrole-3a,4(1H)-diol;
(3aS,5S,6aR)-2-((S)-2-hydroxy-2-(5-hydroxypyridin-2-yl)ethyl)-5-phenoxyhexahydrocyclopenta[c]pyrrol-3a(1H)-ol;
(3aS,5S,6aR)-2-((R)-2-hydroxy-2-(5-hydroxypyridin-2-yl)ethyl)-5-phenoxyhexahydrocyclopenta[c]pyrrol-3a(1H)-ol;
(3aR,5R,6aS)-2-((S)-2-hydroxy-2-(5-hydroxypyridin-2-yl)ethyl)-5-phenoxyhexahydrocyclopenta[c]pyrrol-3a(1H)-ol;
(3aR,5R,6aS)-2-((R)-2-hydroxy-2-(5-hydroxypyridin-2-yl)ethyl)-5-phenoxyhexahydrocyclopenta[c]pyrrol-3a(1H)-ol;
(3aS,5S,6aR)-2-((R)-2-(3,5-difluoro-4-hydroxyphenyl)-2-hydroxyethyl)-5-(2-fluorophenoxy)hexahydrocyclopenta[c]pyrrol-3a(1H)-ol;
(3aS,5S,6aR)-2-((S)-2-(3,5-difluoro-4-hydroxyphenyl)-2-hydroxyethyl)-5-(2-fluorophenoxy)hexahydrocyclopenta[c]pyrrol-3a(1H)-ol;
(3aR,5R,6aS)-2-((R)-2-(3,5-difluoro-4-hydroxyphenyl)-2-hydroxyethyl)-5-(2-fluorophenoxy)hexahydrocyclopenta[c]pyrrol-3a(1H)-ol;
(3aR,5R,6aS)-2-((S)-2-(3,5-difluoro-4-hydroxyphenyl)-2-hydroxyethyl)-5-(2-fluorophenoxy)hexahydrocyclopenta[c]pyrrol-3a(1H)-ol;
(3aS,5S,6aR)-2-((S)-2-(6-fluoro-5-hydroxypyridin-2-yl)-2-hydroxyethyl)-5-phenoxyhexahydrocyclopenta[c]pyrrol-3a(1H)-ol;
(3aS,5S,6aR)-2-((R)-2-(6-fluoro-5-hydroxypyridin-2-yl)-2-hydroxyethyl)-5-phenoxyhexahydrocyclopenta[c]pyrrol-3a(1H)-ol;
(3aR,5R,6aS)-2-((S)-2-(6-fluoro-5-hydroxypyridin-2-yl)-2-hydroxyethyl)-5-phenoxyhexahydrocyclopenta[c]pyrrol-3a(1H)-ol;
(3aR,5R,6aS)-2-((R)-2-(6-fluoro-5-hydroxypyridin-2-yl)-2-hydroxyethyl)-5-phenoxyhexahydrocyclopenta[c]pyrrol-3a(1H)-ol;
(3aS,5S,6aR)-2-((R)-2-(3,5-difluoro-4-hydroxyphenyl)-2-hydroxyethyl)-5-phenoxyhexahydrocyclopenta[c]pyrrol-3a(1H)-ol;
(3aS,5S,6aR)-2-((S)-2-(3,5-difluoro-4-hydroxyphenyl)-2-hydroxyethyl)-5-phenoxyhexahydrocyclopenta[c]pyrrol-3a(1H)-ol;
(3aR,5R,6aS)-2-((R)-2-(3,5-difluoro-4-hydroxyphenyl)-2-hydroxyethyl)-5-phenoxyhexahydrocyclopenta[c]pyrrol-3a(1H)-ol;
(3aR,5R,6aS)-2-((S)-2-(3,5-difluoro-4-hydroxyphenyl)-2-hydroxyethyl)-5-phenoxyhexahydrocyclopenta[c]pyrrol-3a(1H)-ol;
(3aS,5S,6aR)-2-((S)-2-(6-chloro-5-hydroxypyridin-2-yl)-2-hydroxyethyl)-5-phenoxyhexahydrocyclopenta[c]pyrrol-3a(1H)-ol;
(3aS,5S,6aR)-2-((R)-2-(6-chloro-5-hydroxypyridin-2-yl)-2-hydroxyethyl)-5-phenoxyhexahydrocyclopenta[c]pyrrol-3a(1H)-ol;
(3aS,5S,6aR)-2-((R)-2-(3,5-difluoro-4-hydroxyphenyl)-2-hydroxyethyl)-5-(2,4-difluorophenoxy)hexahydrocyclopenta[c]pyrrol-3a(1H)-ol;
(3aS,5S,6aR)-2-((S)-2-(3,5-difluoro-4-hydroxyphenyl)-2-hydroxyethyl)-5-(2,4-difluorophenoxy)hexahydrocyclopenta[c]pyrrol-3a(1H)-ol;
(3aR,5R,6aS)-2-((R)-2-(3,5-difluoro-4-hydroxyphenyl)-2-hydroxyethyl)-5-(2,4-difluorophenoxy)hexahydrocyclopenta[c]pyrrol-3a(1H)-ol;
(3aR,5R,6aS)-2-((S)-2-(3,5-difluoro-4-hydroxyphenyl)-2-hydroxyethyl)-5-(2,4-difluorophenoxy)hexahydrocyclopenta[c]pyrrol-3a(1H)-ol;
(3aS,5S,6aR)-5-(2,4-difluorophenoxy)-2-((R)-2-hydroxy-2-(4-hydroxyphenyl)ethyl)hexahydrocyclopenta[c]pyrrol-3a(1H)-ol;
(3aS,5S,6aR)-5-(2,4-difluorophenoxy)-2-((S)-2-hydroxy-2-(4-hydroxyphenyl)ethyl)hexahydrocyclopenta[c]pyrrol-3a(1H)-ol;
(3aR,5R,6aS)-5-(2,4-difluorophenoxy)-2-((R)-2-hydroxy-2-(4-hydroxyphenyl)ethyl)hexahydrocyclopenta[c]pyrrol-3a(1H)-ol;
(3aR,5R,6aS)-5-(2,4-difluorophenoxy)-2-((S)-2-hydroxy-2-(4-hydroxyphenyl)ethyl)hexahydrocyclopenta[c]pyrrol-3a(1H)-ol;
(3aS,5S,6aR)-2-(3,5-difluoro-4-hydroxyphenethyl)-5-(2-fluorophenoxy)hexahydrocyclopenta[c]pyrrol-3a(1H)-ol;
(3aR,5R,6aS)-2-(3,5-difluoro-4-hydroxyphenethyl)-5-(2-fluorophenoxy)hexahydrocyclopenta[c]pyrrol-3a(1H)-ol;
(3aS,5S,6aR)-2-(3,5-difluoro-4-hydroxyphenethyl)-5-(2,4-difluorophenoxy)hexahydrocyclopenta[c]pyrrol-3a(1H)-ol;
(3aR,5R,6aS)-2-(3,5-difluoro-4-hydroxyphenethyl)-5-(2,4-difluorophenoxy)hexahydrocyclopenta[c]pyrrol-3a(1H)-ol;
(3aS,5S,6aR)-2-(3,5-difluoro-4-hydroxyphenethyl)-5-phenoxyhexahydrocyclopenta[c]pyrrol-3a(1H)-ol;
(3aR,5R,6aS)-2-(3,5-difluoro-4-hydroxyphenethyl)-5-phenoxyhexahydrocyclopenta[c]pyrrol-3a(1H)-ol;
(3aS,5S,6aR)-5-((3-fluoropyridin-2-yl)oxy)-2-(4-hydroxyphenethyl)hexahydrocyclopenta[c]pyrrol-3a(1H)-ol;
(3aR,5R,6aS)-5-((3-fluoropyridin-2-yl)oxy)-2-(4-hydroxyphenethyl)hexahydrocyclopenta[c]pyrrol-3a(1H)-ol;
(3aS,5S,6aR)-5-((2-fluoropyridin-3-yl)oxy)-2-(4-hydroxyphenethyl)hexahydrocyclopenta[c]pyrrol-3a(1H)-ol;
(3aR,5R,6aS)-5-((2-fluoropyridin-3-yl)oxy)-2-(4-hydroxyphenethyl)hexahydrocyclopenta[c]pyrrol-3a(1H)-ol;
(3aS,5S,6aR)-5-(2,4-difluorophenoxy)-2-(2-(5-hydroxypyridin-2-yl)ethyl)hexahydrocyclopenta[c]pyrrol-3a(1H)-ol;
(3aR,5R,6aS)-5-(2,4-difluorophenoxy)-2-(2-(5-hydroxypyridin-2-yl)ethyl)hexahydrocyclopenta[c]pyrrol-3a(1H)-ol;
(3aS,5S,6aR)-5-((2-fluoropyridin-3-yl)oxy)-2-(2-(5-hydroxypyridin-2-yl)ethyl)hexahydrocyclopenta[c]pyrrol-3a(1H)-ol; and
(3aR,5R,6aS)-5-((2-fluoropyridin-3-yl)oxy)-2-(2-(5-hydroxypyridin-2-yl)ethyl)hexahydrocyclopenta[c]pyrrol-3a(1H)-ol, or a pharmaceutically acceptable salt thereof.

One embodiment is a pharmaceutical composition comprising a compound of Formula (I) or a pharmaceutically acceptable salt thereof.

Another embodiment is a method for the treatment of Parkinson's disease, Huntington's disease, amyotrophic lateral sclerosis, multiple sclerosis, seizure disorders, autism, autism spectrum disorders, Fragile X syndrome, tuberous sclerosis, Down's syndrome, bipolar disorder, obsessive-compulsive disorder, anxiety disorder, major depressive disorder, refractory or treatment resistant depression, or suicidality comprising administration of a therapeutically effective amount of a compound of Formula (I), or a pharmaceutically acceptable salt thereof to a patient in need of treatment thereof.

Another embodiment is a method for the treatment of post-traumatic stress disorder (PTSD).

Another embodiment is a method for the treatment of cocaine use disorder.

Another embodiment is a method for the treatment of pain and migraine.

Another embodiment is a method for the treatment of Rett Syndrome.

Another embodiment is a method for the treatment of tinnitus.

Unless specified otherwise, the term “compounds of the present disclosure” or “compound of the present disclosure” refers to compounds of formula (I) subformulae thereof, and exemplified compounds, and salts thereof, as well as all stereoisomers (including diastereoisomers and enantiomers), rotamers, tautomers and isotopically labeled compounds (including deuterium substitutions), as well as inherently formed moieties.

### Definitions

As used herein, the terms “Halogen”, “halide”, or, alternatively, “halo” refer to bromo, chloro, fluoro or iodo.

As used herein, the term “C₁₋₆alkyl” refers to a straight or branched hydrocarbon chain radical consisting solely of carbon and hydrogen atoms, containing no unsaturation, having from one to six carbon atoms, and which is attached to the rest of the molecule by a single bond. The term “C₁₋₄alkyl” is to be construed accordingly. Examples of C₁₋₆alkyl include, but are not limited to, methyl, ethyl, n-propyl, 1-methylethyl (iso-propyl), n-butyl, n-pentyl and 1,1-dimethylethyl (t-butyl).

As used herein, the term “C₃₋₈cycloalkyl” refers to a monocyclic or polycyclic radical that contains only carbons and hydrogen, having from three to eight ring atoms, and can be saturated or partially unsaturated. Examples of C₃₋₈cycloalkyl include, but are not limited to cyclopropyl, cyclobutyl, cyclopentyl, cyclopentyenyl, cyclohexyl, cycloheptyl, and cyclooctyl.

As used herein, the term “hydroxyC₁₋₆alkyl” refers to a C₁₋₆alkyl radical as defined above, wherein one of the hydrogen atoms of the C₁₋₆alkyl radical is replaced by OH. Examples of hydroxyC₁₋₆alkyl include, but are not limited to, hydroxy-methyl, 2-hydroxy-ethyl, 2-hydroxy-propyl, 3-hydroxy-propyl and 5-hydroxy-pentyl.

As used herein, the term “haloC₁₋₆alkyl” refers to C₁₋₆alkyl radical, as defined above, substituted by one or more halo radicals, as defined above. Examples of halo C₁₋₆alkyl include, but are not limited to, trifluoromethyl, difluoromethyl, fluoromethyl, trichloromethyl, 2,2,2-trifluoroethyl, 1,3-dibromopropan-2-yl, 3-bromo-2-fluoropropyl and 1,4,4-trifluorobutan-2-yl.

As used herein, the term “Aryl” refers to an aromatic hydrocarbon ring system. Aryl groups are monocyclic ring systems or bicyclic ring systems. Monocyclic aryl ring refers to phenyl. Bicyclic aryl rings refer to naphthyl. Aryl groups may be optionally substituted with one or more substituents as defined in formula (I).

As used herein, the term “Heterocyclic” or “heterocyclyl” refers to a 3 to 8 membered saturated or partially unsaturated monocyclic or bicyclic ring containing from 1 to 5 heteroatoms. Heterocyclic ring systems are not aromatic. Heterocyclic groups containing more than one heteroatom may contain different heteroatoms. Heterocyclic includes ring systems wherein a carbon atom is oxidized forming a cyclic ketone or lactam group. Heterocyclic also includes ring systems wherein a sulfur atom is oxidized to form SO or SO₂. Heterocyclic groups may be optionally substituted with one or more substituents as defined in formula (I). Heterocyclic groups are monocyclic, spiro, or fused or bridged bicyclic ring systems. Monocyclic heterocyclic have 3 to 7 ring atoms, unless otherwise defined. Examples of monocyclic heterocyclic groups include tetrahydrofuranyl, dihydrofuranyl, 1,4-dioxanyl, morpholinyl, 1,4-dithianyl, piperazinyl, piperidinyl, 1,3-dioxolanyl, imidazolidinyl, imidazolinyl, pyrrolinyl, pyrrolidinyl, tetrahydropyranyl, dihydropyranyl, oxathiolanyl, dithiolanyl, 1,3-dioxanyl, 1,3-dithianyl, oxathianyl, thiomorpholinyl and the like. Fused heterocyclic ring systems have from 8 to 11 ring atoms and include groups wherein a heterocyclic ring is fused to a phenyl or monocyclic heteroaryl ring. Examples of fused heterocyclic rings include 3,4-dihydroquinolin-2(1H)-onyl and the like.

As used herein, the term “Heteroaryl” refers to an aromatic ring system containing from 1 to 5 heteroatoms. Heteroaryl groups containing more than one heteroatom may contain different heteroatoms. Heteroaryl groups may be optionally substituted with one or more substituents as defined in formula (I). Heteroaryl groups are monocyclic ring systems or are fused bicyclic ring systems. Monocyclic heteroaryl rings have from 5 to 6 ring atoms. Bicyclic heteroaryl rings have from 8 to 10 member atoms. Heteroaryl includes, but is not limited to, pyrrolyl, pyrazolyl, imidazolyl, oxazolyl, isoxazolyl, oxadiazolyl, thiazolyl, isothiazolyl, thiadiazolyl, furanyl, furanzanyl, thienyl, triazolyl, pyridinyl, pyrimidinyl, pyridazinyl, trazinyl, tetrazinyl, tetrzolyl, indonyl, isoindolyl, indolizinyl, indazolyl, purinyl, quinolinyl, isoquinolinyl, quinoxalinyl, quinazolinyl, benzimidazolyl, benzopyranyl, benzopyranyl, benzoxazolyl, benzoisoxazolyl, benzofuranyl, benzothiazolyl, benzothienyl, and naphthyridinyl.

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present disclosure is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present disclosure. “Salts” include in particular “pharmaceutical acceptable salts”. The term “pharmaceutically acceptable salts” refers to salts that retain the biological effectiveness and properties of the compounds of this disclosure and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present disclosure provides compounds of the present disclosure in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the disclosure include, for example, isotopes of hydrogen.

For example, Formula (III) is deuterated as shown in the compound of formula (IIIg):

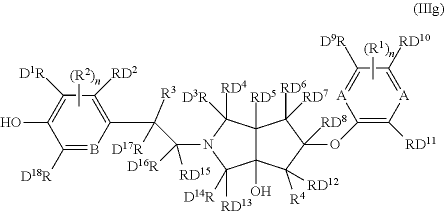

or a pharmaceutically acceptable salt thereof, wherein R¹, R², and n are defined as in Formula (I), RD¹ through RD¹⁸ are independently H or D, and R³, R⁴ are independently H, D, or OH, each A is independently N, CH, or CD, provided that when one A is N the other A is CH or CD; B is N, CH, or CD.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present disclosure. The concentration of deuterium, may be defined by the isotopic enrichment factor. The term “isotopic enrichment factor” as used herein means the ratio between the isotopic abundance and the natural abundance of a specified isotope. If a substituent in a compound of this disclosure is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the disclosure include isotopes of hydrogen, carbon, nitrogen, oxygen, phosphorous, fluorine, and chlorine, such as ³H, ¹¹C, ¹³C, ¹⁴C, ¹⁵N, ¹⁸F ³¹P, ³²P, ³⁵S, ³⁶Cl, ¹²³I, ¹²⁴I, ¹²⁵I respectively. Accordingly it should be understood that the disclosure includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present disclosure can generally be prepared by conventional techniques known to those skilled in the art or by processes analogous to those described in the accompanying Examples and Preparations using an appropriate isotopically-labeled reagent in place of the non-labeled reagent previously employed.

As used herein, the term “pharmaceutical composition” refers to a compound of the disclosure, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

The term “a therapeutically effective amount” of a compound of the present disclosure refers to an amount of the compound of the present disclosure that will elicit the biological or medical response of a subject, for example, reduction or inhibition of an enzyme, receptor, ion channel, or a protein activity, or ameliorate symptoms, alleviate conditions, slow or delay disease progression, or prevent a disease, etc. In one embodiment, the term “a therapeutically effective amount” refers to the amount of the compound of the present disclosure that, when administered to a subject, is effective to (1) at least partially alleviate, prevent and/or ameliorate a condition, or a disorder or a disease (i) mediated by NR2B receptor, or (ii) associated with NR2B receptor activity, or (iii) characterized by activity (normal or abnormal) of NR2B receptor; or (2) reduce or inhibit the activity of NR2B receptor; or (3) reduce or inhibit the expression of NR2B receptor. In another embodiment, the term “a therapeutically effective amount” refers to the amount of the compound of the present disclosure that, when administered to a cell, or a tissue, or a non-cellular biological material, or a medium, is effective to at least partially reducing or inhibiting the activity of NR2B receptor; or at least partially reducing or inhibiting the expression of NR2B receptor. The meaning of the term “a therapeutically effective amount” as illustrated in the above embodiment for NR2B receptor also applies by the same means to any other relevant proteins/peptides/enzymes/receptors/ion channels, such as NMDA receptor, and the like.

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder

As used herein, a subject is “in need of” a treatment if such subject would benefit biologically, medically or in quality of life from such treatment.

As used herein, the term “a,” “an,” “the” and similar terms used in the context of the present disclosure (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the disclosure otherwise claimed.

Any asymmetric atom (e.g., carbon or the like) of the compound(s) of the present disclosure can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present disclosure can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present disclosure or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present disclosure or racemic intermediates can also be resolved by chiral chromatography, e.g., high pressure liquid chromatography (HPLC) using a chiral adsorbent.

The disclosure further includes any variant of the present processes, in which an intermediate obtainable at any stage thereof is used as starting material and the remaining steps are carried out, or in which the starting materials are formed in situ under the reaction conditions, or in which the reaction components are used in the form of their salts or optically pure material. Compounds of the present disclosure and intermediates can also be converted into each other according to methods generally known to those skilled in the art.

Pharmaceutical Compositions

In another aspect, the present disclosure provides a pharmaceutical composition comprising a compound of the present disclosure, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. Topical administration may also pertain to inhalation or intranasal application. The pharmaceutical compositions of the present disclosure can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Methods of Use

The compounds of the present disclosure in free form or in pharmaceutically acceptable salt form, exhibit valuable pharmacological properties, e.g. NR2B receptor modulating properties, for example as negative allosteric modulators of the NR2B receptor, e.g. as indicated in vitro and in vivo tests as provided in the next sections, and are therefore indicated for therapy or for use as research chemicals, e.g. as tool compounds.

Compounds of the present disclosure may be useful in the treatment of an indication selected from: Parkinson's disease, Huntington's disease, Rett syndrome, amyotrophic lateral sclerosis, multiple sclerosis, seizure disorders, autism, autism spectrum disorders, Fragile X syndrome, tuberous sclerosis, Down's syndrome, pain, migraine, tinnitus, bipolar disorder, obsessive-compulsive disorder, anxiety disorder, post-traumatic stress disorder (PTSD), cocaine use disorder, major depressive disorder, refractory or treatment resistant depression, or suicidality. Specifically compounds of the present disclosure may be useful in the treatment of an indication selected from: major depressive disorder, refractory or treatment resistant depression, and suicidality.

Thus, as a further aspect, the present disclosure provides the use of a compound of the present disclosure or a pharmaceutically acceptable salt thereof in therapy. In a further embodiment, the therapy is selected from a disease which may be treated by negative allosteric modulation of NR2B receptor. In another embodiment, the disease is selected from the afore-mentioned list.

Thus, as a further aspect, the present disclosure provides the use of a compound of the present disclosure or a pharmaceutically acceptable salt thereof, for the manufacture of a medicament. In a further embodiment, the medicament is for treatment of a disease which may be treated by negative allosteric modulation of NR2B receptor. In another embodiment, the disease is selected from the afore-mentioned list.

In one embodiment of the present disclosure, there is provided the compound of Formula (I) for use in the treatment of Parkinson's disease, Huntington's disease, Rett syndrome, amyotrophic lateral sclerosis, multiple sclerosis, seizure disorders, autism, autism spectrum disorders, Fragile X syndrome, tuberous sclerosis, Down's syndrome, pain, migraine, tinnitus, bipolar disorder, obsessive-compulsive disorder, anxiety disorder, post-traumatic stress disorder (PTSD), cocaine use disorder, major depressive disorder, refractory or treatment resistant depression, or suicidality. Specifically there is provided the compound of Formula (I) for use in the treatment of major depressive disorder, refractory or treatment resistant depression, or suicidality.

The pharmaceutical composition or combination of the present disclosure can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present disclosure can be applied in vitro in the form of solutions, e.g., aqueous solutions, and in vivo either internally, parenterally, advantageously intravenously, e.g., as a suspension or in aqueous solution. The dosage in vitro may range between about 10-3 molar and 10-9 molar concentrations. A therapeutically effective amount in vivo may range depending on the route of administration, between about 0.1-500 mg/kg, or between about 1-100 mg/kg.

Combinations

“Combination” refers to either a fixed combination in one dosage unit form, or a combined administration where a compound of the present disclosure and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present disclosure and a combination partner, are both administered to a patient simultaneously in the form of a single entity or dosage. The term “non-fixed combination” means that the therapeutic agents, e.g. a compound of the present disclosure and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The compound of the present disclosure may be administered either simultaneously with, or before or after, one or more other therapeutic agent. The compound of the present disclosure may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the present disclosure.

In one embodiment, the disclosure provides a product comprising a compound of the present disclosure and at least one other therapeutic agent as a combined preparation for simultaneous, separate or sequential use in therapy. In one embodiment, the therapy is the treatment of a disease or condition mediated by negative allosteric modulation of NR2B receptor. Products provided as a combined preparation include a composition comprising the compound of the present disclosure and the other therapeutic agent(s) together in the same pharmaceutical composition, or the compound of the present disclosure and the other therapeutic agent(s) in separate form, e.g. in the form of a kit.

In one embodiment, the disclosure provides a pharmaceutical composition comprising a compound of the present disclosure and another therapeutic agent(s). Optionally, the pharmaceutical composition may comprise a pharmaceutically acceptable carrier, as described above.

In one embodiment, the disclosure provides a kit comprising two or more separate pharmaceutical compositions, at least one of which contains a compound of the present disclosure. In one embodiment, the kit comprises means for separately retaining said compositions, such as a container, divided bottle, or divided foil packet. An example of such a kit is a blister pack, as typically used for the packaging of tablets, capsules and the like. The kit of the disclosure may be used for administering different dosage forms, for example, oral and parenteral, for administering the separate compositions at different dosage intervals, or for titrating the separate compositions against one another. To assist compliance, the kit of the disclosure typically comprises directions for administration.

In the combination therapies of the disclosure, the compound of the present disclosure and the other therapeutic agent may be manufactured and/or formulated by the same or different manufacturers. Moreover, the compound of the present disclosure and the other therapeutic may be brought together into a combination therapy: (i) prior to release of the combination product to physicians (e.g. in the case of a kit comprising the compound of the present disclosure and the other therapeutic agent); (ii) by the physician themselves (or under the guidance of the physician) shortly before administration; (iii) in the patient themselves, e.g. during sequential administration of the compound of the present disclosure and the other therapeutic agent.

Accordingly, the disclosure provides the use of a compound of the present disclosure for treating a disease or condition mediated by negative allosteric modulation of NR2B receptor, wherein the medicament is prepared for administration with another therapeutic agent. The disclosure also provides the use of another therapeutic agent for treating a disease or condition mediated by negative allosteric modulation of NR2B receptor, wherein the medicament is administered with a compound of the present disclosure.

The disclosure also provides a compound of the present disclosure for use in a method of treating a disease or condition mediated by negative allosteric modulation of NR2B receptor, wherein the compound of the present disclosure is prepared for administration with another therapeutic agent. The disclosure also provides another therapeutic agent for use in a method of treating a disease or condition mediated by negative allosteric modulation of NR2B receptor, wherein the other therapeutic agent is prepared for administration with a compound of the present disclosure. The disclosure also provides a compound of the present disclosure for use in a method of treating a disease or condition mediated by negative allosteric modulation of NR2B receptor, wherein the compound of the present disclosure is administered with another therapeutic agent. The disclosure also provides another therapeutic agent for use in a method of treating a disease or condition mediated by negative allosteric modulation of NR2B receptor, wherein the other therapeutic agent is administered with a compound of the present disclosure.

The disclosure also provides the use of a compound of the present disclosure for treating a disease or condition mediated by NR2B receptor, wherein the patient has previously (e.g. within 24 hours) been treated with another therapeutic agent. The disclosure also provides the use of another therapeutic agent for treating a disease or condition mediated by NR2B receptor, wherein the patient has previously (e.g. within 24 hours) been treated with compound of the present disclosure.

In one embodiment, the other therapeutic agent is selected from:
(a) lithium;
(b) stimulants, such as amphetamine and dextroamphetamine, (Adderall™) or methylphenidate Italin™);
(c) acetylcholinesterase inhibitors, such as donepezil (Aricept™), rivastigmine (Exelon™) and galantamine (Razadyne™);
(d) antidepressant medications for low mood and irritability, such as citalopram (Celexa™) fluoxetine (Prozac™), paroxeine (Paxil™), sertraline (Zoloft™), trazodone (Desyrel™), and tricyclic antidepressants such as amitriptyline (Elavil™);
(e) anxiolytics for anxiety, restlessness, verbally disruptive behavior and resistance, such as lorazepam (Ativan™) and oxazepam (Serax™);
(f) antipsychotic medications for hallucinations, delusions, aggression, agitation, hostility and uncooperativeness, such as aripiprazole (Abilify™), clozapine (Clozaril™), haloperidol (Haldol™), olanzapine (Zyprexa™), quetiapine (Seroquel™), risperidone (Risperdal™) and ziprasidone (Geodon™);
(g) mood stabilizers, such as carbamazepine (Tegretol™) and divalproex (Depakote™); (h) pregabalin;
(i) gabapentin (Neurontin™);
(j) dopamine agonists such as L-DOPA, pramipexole (Mirapex™) and ropinerol (Requip™);
(k) analgesics including opiates and non-opiates;
(k) carbidopa;
(l) triptans such as sumatriptan (Imitrex™) and zolmitriptan (Zomig™);
(m) nicotinic apha-7 agonists;
(n) mGluR5 antagonists;
(o) H3 agonists;
(p) amyloid therapy vaccines; and
(q) chemotherapy agents.

In one embodiment of the disclosure, there is provided a product comprising a NR2B modulator and aforementioned combination partners as a combined preparation for simultaneous, separate or sequential use in therapy.

In another embodiment of the disclosure, there is provided a product comprising a NR2B modulator and aforementioned combination partners as a combined preparation for simultaneous, separate or sequential use in therapy.

In one embodiment of the disclosure, there is provided a pharmaceutical composition comprising a NR2B modulator, aforementioned combination partners, and a pharmaceutically acceptable carrier.

In a further embodiment of the disclosure, there is provided a pharmaceutical composition comprising a NR2B modulator, aforementioned combination partners, and a pharmaceutically acceptable carrier.

### Preparation of Compounds

Compounds of the present disclosure can be prepared as described in the following Examples.

Intermediates described herein can be prepared as shown in Scheme 1 below.

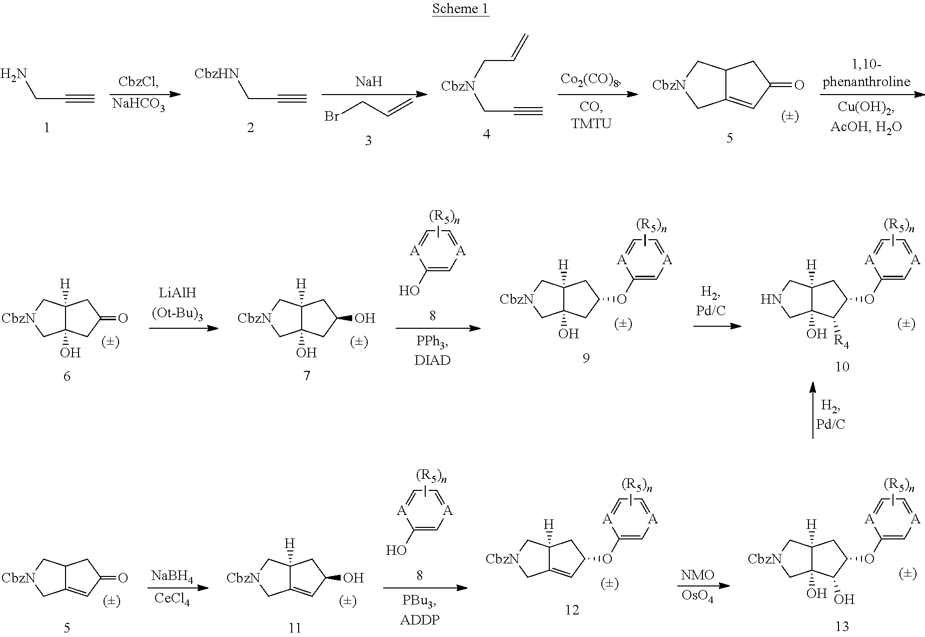

In Scheme 1, propargylamine 1 can be treated with benzyl chloroformate to give protected amine 2, which can then be allylated with allyl bromide to provide 4. This can undergo a Pauson-Khand cycloaddition to provide the bicyclic enone 5. This key intermediate can be oxidized at the bridgehead position to give cis-fused alcohol 6, which can be reduced to diol 7 with control of the relative stereochemistry. The Mitsunobu reaction with a phenol such as 8 (where R₅, n, and A are as defined in the claims) proceeds with inversion of stereochemistry, generating the desired all-cis configuration of an ether such as 9, which can be deprotected by hydrogenation to yield a free amine such as 10 (where R₄ is H). Alternatively, 5 can first be reduced under Luche conditions to allylic alcohol 11. The Mitsunobu-type reaction with a phenol such as 8 now gives an olefin such as 12, which can be subjected to dihydroxylation with osmium tetroxide, providing a diol such as 13. As before, hydrogenation of the protecting group can give a free amine such as 10 (where R₄ is OH). This can either be brought forward as a racemic mixture, or intermediates 7 or 13 can be chirally separated into their enantiomers, which can be brought separately through the rest of the sequence.

Compounds provided herein can be prepared as shown in Scheme 2 below.

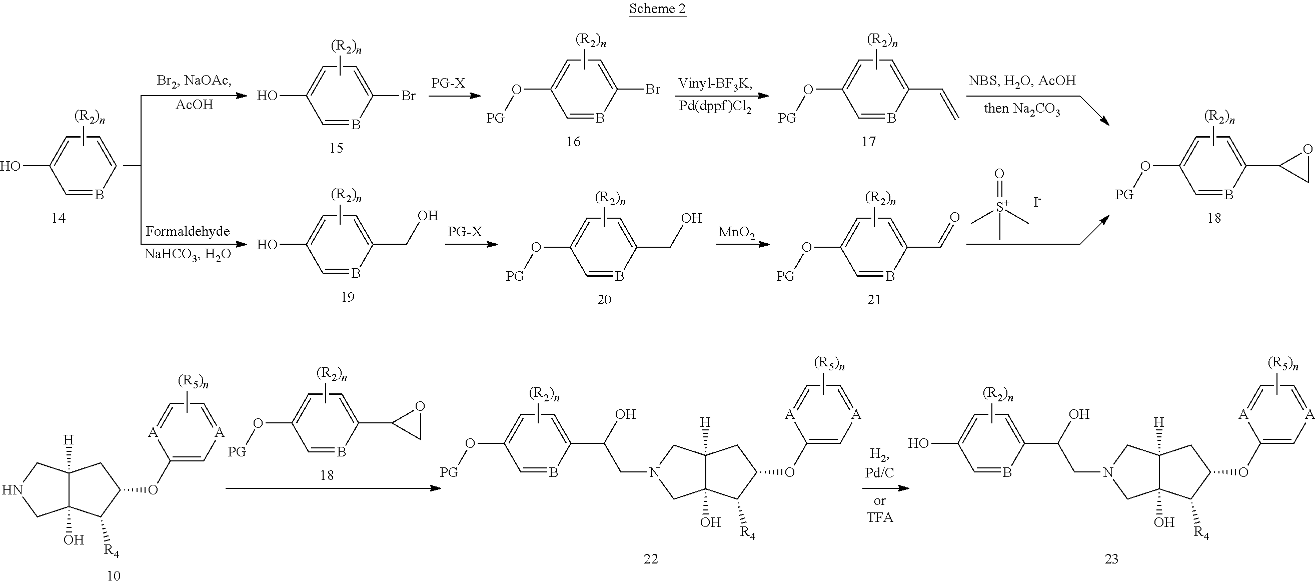

In Scheme 2, a phenol such as 14 (where R₂, n, and B are as defined in the claims) can be treated with a brominating agent such as bromine to give 15, which can be protected using reagents such as benzyl bromide or 4-methoxybenzyl chloride to give a protected phenol such as 16. This can undergo Suzuki-Miyaura coupling with potassium vinyltrifluoroborate in the presence of a palladium catalyst and base to yield an olefin such as 17, which can be converted to an epoxide such as 18 with a brominating reagent such as N-bromosuccinimide under aqueous conditions. Alternatively, 14 can be treated with formaldehyde under basic aqueous conditions to provide an alcohol such as 19, which can be protected selectively at the phenol position as before to give a protected phenol such as 20. The free primary alcohol can be oxidized with an oxidizing agent such as manganese(IV) oxide to give an aldehyde such as 21, which can then be treated with an ylide formed from a sulfoxonium halide such as trimethylsulfoxonium iodide and a base such as sodium hydride to generate an epoxide such as 18. This epoxide can be opened with an amine such as 10 (where R₄, R₅, n, and A are as defined in the claims) to give amino-alcohols such as 22, which can be deprotected by hydrogenation (for benzyl and p-methoxybenzyl protecting groups) or by treatment with an acid such as trifluoroacetic acid (for p-methoxybenzyl) to provide examples such as 23, which can be separated into its single diastereomers by chiral chromatography.

Alternatively, compounds provided herein can be prepared as shown in Scheme 3 below.

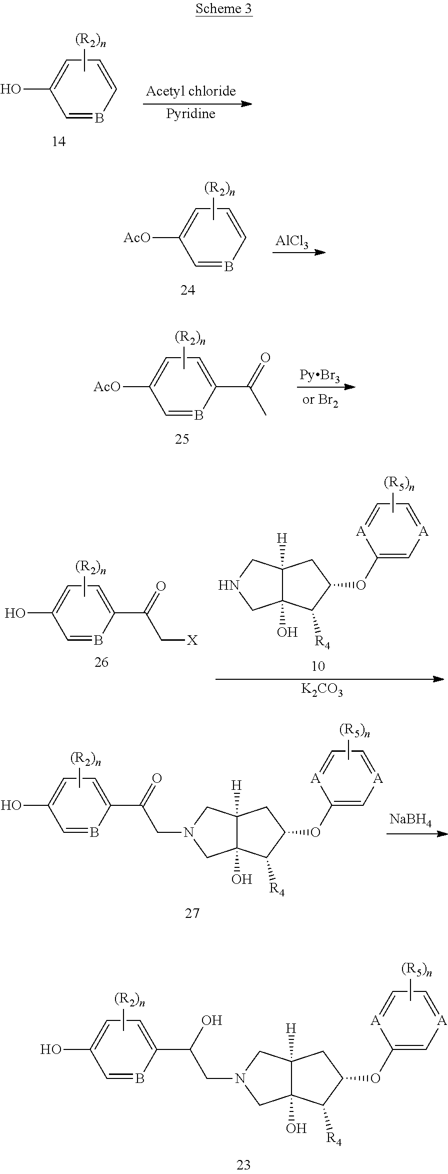

In Scheme 3, a phenol such as 14 (where R₂, n, and B are as defined in the claims) can react with acetyl chloride in the presence of a base such as pyridine to provide an acetate such as 24, which can be treated with a Lewis acid such as aluminum chloride with heating to trigger a rearrangement to a ketone such as 25. This can be treated with a halogenating agent such as pyridinium tribromide or bromine to form an α-haloketone such as 26, which can undergo a nucleophilic displacement with an amine such as 10 (where R₄, R₅, n, and A are as defined in the claims) in the presence of a base such as potassium carbonate to yield a ketone such as 27. This can be reduced with a reducing agent such as sodium borohydride to provide examples such as 23, which can be separated into its single diastereomers by chiral chromatography.

Alternatively, compounds provided herein can be prepared as shown in Scheme 4 below.

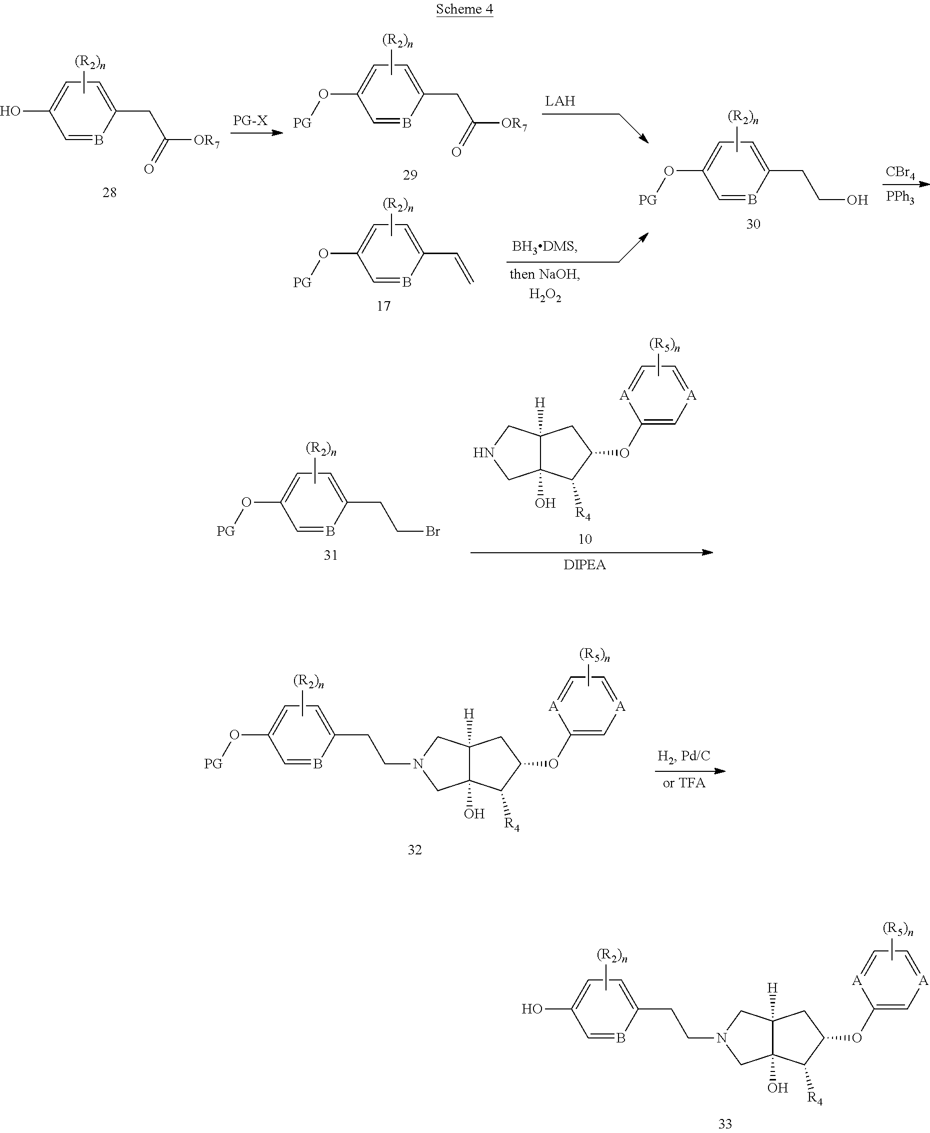

In Scheme 4, a phenol with a pendant ester group such as 28 (where R₂, n, and B are as defined in the claims, and R₇ is an alkyl group such as methyl or ethyl) can be protected using reagents such as benzyl bromide or 4-methoxybenzyl chloride to give a protected phenol such as 29. The ester can be reduced with a reducing agent such as lithium aluminum hydride to give a primary alcohol such as 30, which can be converted to the bromide 31 with a brominating agent such as carbon tetrabromide in the presence of triphenylphosphine. Alternatively, alcohol 30 can be generated by subjecting an olefin such as 17 to a hydroboration/oxidation sequence. The bromide of 31 can be displaced by an amine such as 10 in the presence of a base such as diisopropylethylamine to provide compounds such as 32, which can be deprotected by hydrogenation (for benzyl and p-methoxybenzyl protecting groups) or by treatment with an acid such as trifluoroacetic acid (for p-methoxybenzyl) to provide examples such as 33, which can be separated into its single enantiomers by chiral chromatography.

Alternatively, compounds provided herein can be prepared as shown in Scheme 5 below.

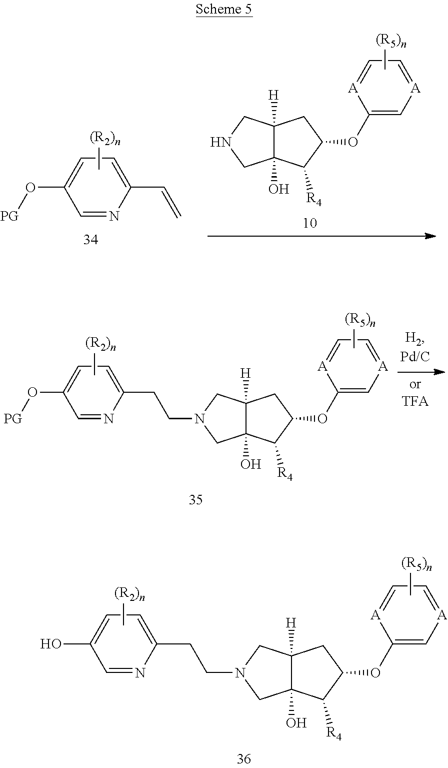

In Scheme 5, olefin 34 (equivalent to compound 17 where B is N) can be reacted with amine 10 to provide compounds such as 35, which can be deprotected by hydrogenation (for benzyl and p-methoxybenzyl protecting groups) or by treatment with an acid such as trifluoroacetic acid (for p-methoxybenzyl) to provide examples such as 36, which can be separated into its single enantiomers by chiral chromatography.

### Intermediates and Examples

The following examples are intended to illustrate the disclosure and are not to be construed as being limitations thereon.

Many examples were made as mixtures of two or four stereoisomers, then separated into single isomers which were tested individually in the NR2B rat cortical neuron calcium influx assay described in the Biological Data section below. However, the stereochemistry of every enantiomer was not determined. The stereochemistry of Example 10D was determined by single crystal x-ray crystallography to be (3aS,5S,6aR)-2-((S)-2-(3,5-difluoro-4-hydroxyphenyl)-2-hydroxyethyl)-5-phenoxyhexahydrocyclopenta[c]pyrrol-3a(1H)-ol, as depicted below.

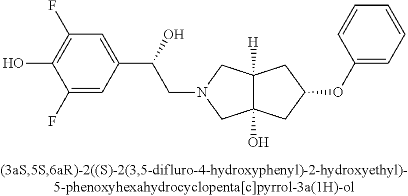 [SMILES: Oc1c(F)cc([C@H](O)CN2C[C@H]3C[C@H](Oc4ccccc4)C[C@@]3(O)C2)cc1F]

From this crystal structure, structure-activity relationship analysis, chemical correlation, and knowledge from WO 2016/049165 A1, it is assumed that the (3aS,5S,6aR) configuration of the hexahydrocyclopenta[c]pyrrole core [or the (3aS,4S,5S,6aR) configuration when R₄ is OH] is more active than the (3aR,5R,6aS) configuration [or the (3aR,4R,5R,6aS) configuration when R₄ is OH] in all of the Examples. Although there is strong evidence to suggest that the (3aS,5S,6aR) [or (3aS,4S,5S,6aR)] configuration is the more active configuration, there is still the chance that the (3aR,5R,6aS) [or (3aR,4R,5R,6aS)] configuration could be the more active configuration in some of the Examples.

Within sets of Examples where the stereochemistry of each Example has not been fully determined, the possible names and chemical structures have been listed according to their structural orientation. Generally, compounds containing the (3aS,5S,6aR) [or (3aS,4S,5S,6aR)] core have been listed before compounds containing the (3aR,5R,6aS) [or (3aR,4R,5R,6aS)] core, and compounds where the benzylic alcohol is in the “up” orientation as drawn have been listed before compounds where the benzylic alcohol is in the “down” orientation as drawn (note that using R/S notation at this position is not always consistent, as the prioritization of groups around this chiral center will change based on whether B, as defined in the claims, corresponds to CH or N). This order does not necessarily correspond to the NB or NB/C/D order within that set of Examples (the NB or NB/C/D order generally refers to the order that the compounds were obtained from chiral separation).

For illustration, within the set of Examples 10A/10B/10C/10D, the four possible names and chemical structures are listed as follows:
(3aS,5S,6aR)-2-((R)-2-(3,5-difluoro-4-hydroxyphenyl)-2-hydroxyethyl)-5-phenoxyhexahydrocyclopenta[c]pyrrol-3a(1H)-ol
(3aS,5S,6aR)-2-((S)-2-(3,5-difluoro-4-hydroxyphenyl)-2-hydroxyethyl)-5-phenoxyhexahydrocyclopenta[c]pyrrol-3a(1H)-ol
(3aR,5R,6aS)-2-((R)-2-(3,5-difluoro-4-hydroxyphenyl)-2-hydroxyethyl)-5-phenoxyhexahydrocyclopenta[c]pyrrol-3a(1H)-ol
(3aR,5R,6aS)-2-((S)-2-(3,5-difluoro-4-hydroxyphenyl)-2-hydroxyethyl)-5-phenoxyhexahydrocyclopenta[c]pyrrol-3a(1H)-ol

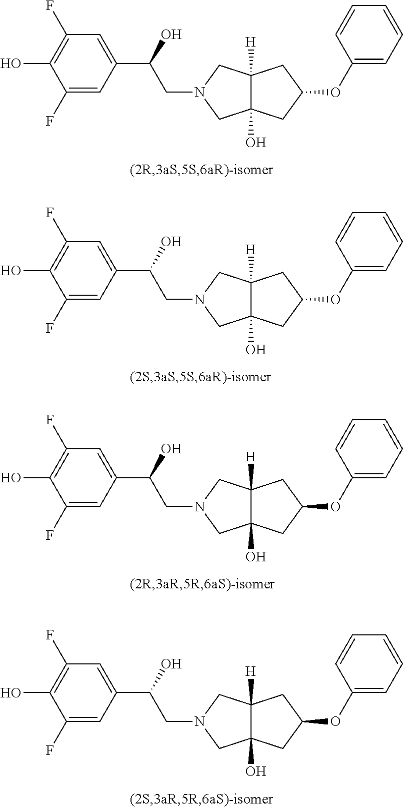 [SMILES: Oc1c(F)cc([C@@H](O)CN2C[C@H]3C[C@H](Oc4ccccc4)C[C@@]3(O)C2)cc1F ; Oc1c(F)cc([C@H](O)CN2C[C@H]3C[C@H](Oc4ccccc4)C[C@@]3(O)C2)cc1F ; Oc1c(F)cc([C@@H](O)CN2C[C@@H]3C[C@@H](Oc4ccccc4)C[C@]3(O)C2)cc1F ; Oc1c(F)cc([C@H](O)CN2C[C@@H]3C[C@@H](Oc4ccccc4)C[C@]3(O)C2)cc1F]

In this case, although Example 10D has been determined by x-ray crystallography to be (3aS,5S,6aR)-2-((S)-2-(3,5-difluoro-4-hydroxyphenyl)-2-hydroxyethyl)-5-phenoxyhexahydrocyclopenta[c]pyrrol-3a(1H)-ol, its name and structure are still listed second out of the four within this set, in accordance with the ordering system used throughout the Examples.

### Abbreviations

Abbreviations used are those conventional in the art or the following:
Ac acetyl
ACN acetonitrile
AcOH acetic acid
ADDP 1,1′-(azodicarbonyl)dipiperidine
aq aqueous
atm atmosphere
BINAP 2,2′-bis(diphenylphosphino)-1,1′-binaphthyl
Bn benzyl
Boc tert-butyloxycarbonyl
Bu butyl
B₂(pin)₂ bis(pinacolato)diboron
C Celsius
Cbz carboxybenzyl
DCM dichloromethane
DEA diethylamine
DIAD diisopropyl azodicarboxylate
DIPEA N,N-diisopropylethylamine
DMF N,N-dimethylformamide
DMS dimethylsulfide
DMSO dimethylsulfoxide
dppf 1,1′-bis(diphenylphosphino)ferrocene
EtOAc ethyl acetate
EtOH ethanol
Et₂O diethyl ether
FCC flash column chromatography
g gram(s)
h hour(s)
HBSS Hanks' balanced salt solution
HPLC high performance liquid chromatography
IACUC Institutional Animal Care and Use Committee
IC₅₀ half maximal inhibitory concentration
IPA isopropyl alcohol
L liter(s)
LAH lithium aluminum hydride
LCMS liquid chromatography and mass spectrometry
Me methyl
MeOH methanol
mg milligram(s)
MHz megahertz
min minute(s)
mL milliliter(s)
mm millimeter(s)
mM millimolar
mmol millimole(s)
MS mass spectrometry
MTBE methyl tert-butyl ether
m/z mass to charge ratio
NaOAc sodium acetate
NADPH nicotinamide adenine dinucleotide phosphate
NBS N-bromosuccinimide
nm nanometer(s)
nM nanomolar
NMO N-methylmorpholine N-oxide
NMR nuclear magnetic resonance
Pd/C palladium on carbon
PE petroleum ether
PG protecting group
Ph phenyl
PMB para-methoxybenzyl
ppm parts per million
Py pyridine; pyridinium
rac racemic
Rt retention time
RT room temperature
SFC supercritical fluid chromatography
t-Bu tert-butyl
TEA triethylamine
TFA trifluoroacetic acid
THF tetrahydrofuran
TLC thin-layer chromatography
TMTU N,N,N,N-tetramethylthiourea
μL microliter(s)
μm micrometer(s); micron(s)
μM micromolar
UPLC ultra performance liquid chromatography
UV ultraviolet

General Procedures

Where no preparative route is described, the material is commercially available. Commercial reagents were used without additional purification unless otherwise stated. Room temperature (RT) is approximately 20-25° C. ¹H NMR were recorded on a 300 MHz Varian, a 400 MHz Varian or a 400 MHz Bruker NMR instrument. Chemical shifts are reported as parts per million (ppm) relative to tetramethylsilane and coupling constants (J) are reported in Hertz. Abbreviations for multiplicity are: s=singlet, d=doublet, t=triplet, q=quartet, dd=doublet of doublet, dt=doublet of triplet, br=broad.

LCMS Method A:

Instrument: Waters Acquity UPLC, photodiode array detector; Column: AcQuity UPLC BEH C₁₈, 1.7 μm, 2.1×30 mm; 2 min run time, 2% solvent B from 0 to 0.1 min, 2→98% solvent B from 0.1 to 1.8 min, 2% solvent B for 0.2 min. Solvents: Solvent A=0.1% formic acid in water (v/v), solvent B=0.1% formic acid in acetonitrile (v/v). Injection volume 2-5 uL; UV detection array 210-400 nm; mass detection 120-1250 (electrospray ionization); column at 50° C.; flow rate 1.0 mL/min.

LCMS Method B:

Instrument: Waters Acquity UPLC, photodiode array detector; Column AcQuity UPLC BEH C₁₈ 1.7 μm 21×30 mm; 5.2 min run time, 2→98% solvent B from 0 to 5.15 min, 98% solvent B from 5.15 to 5.20 min. Solvents: Solvent A=0.1% formic acid in water (v/v), solvent B=0.1% formic acid in acetonitrile (v/v). Injection volume 2-5 uL; UV detection array 210-400 nm; mass detection 120-1600; column at 50° C., flow rate 1.0 mL/min.

LCMS Method C:

Instrument: Waters Acquity UPLC, photodiode array detector; Column: AcQuity UPLC BEH C₁₈, 1.7 μm, 21×30 mm; 1.2 min run time, 2% solvent B from 0 to 0.1 min, 2→80% solvent B from 0.1 to 0.5 min, 80→95% solvent B from 0.5 to 0.6 min, 95% solvent B from 0.6 to 0.8 min, 95→2% solvent B from 0.8 to 0.9 min, 2% solvent B from 0.9 to 1.20 min. Solvents: Solvent A=0.05% formic acid in water (v/v), solvent B=0.04% formic acid in methanol (v/v). UV detection array 200-300 nm; mass detection 100-1600 (electrospray ionization); column at 55° C.; flow rate 1.0 mL/min.

LCMS Method D:

Instrument: API 2000, photodiode array detector; Column: Synergi 2.5 micron MAX-RP 100 A Mercury; 3.0 min run time, 30% solvent B from 0 to 0.5 min, 30→95% solvent B from 0.5 to 1.5 min, 95% solvent B from 1.5 to 2.4 min, 95→30% solvent B from 2.4 to 2.5 min, 30% solvent B from 2.5 to 3.0 min. Solvents: Solvent A=0.1% formic acid in water (v/v), solvent B=acetonitrile. UV detection array 190-400; Mass detection 100-1000 (electrospray ionization); Column at 30° C.; flow rate 2.0 mL/min.

LCMS Method E:

Instrument: API 2000, photodiode array detector; Column: Synergi 2.5 micron MAX-RP 100 A Mercury; 4.0 min run time, 20→50% solvent B from 0.0 to 0.2 min, 50→95% solvent B from 0.2 to 1.0 min, 95% solvent B from 1.0 to 2.5 min, 95→50% solvent B from 2.5 to 2.9 min, 50→20% solvent B from 2.9 to 3.2 min, 20% solvent B from 3.2 to 4.0 min. Solvents: Solvent A=0.1% formic acid in water (v/v), solvent B=acetonitrile. UV detection array 190-400; Mass detection 100-1000 (electrospray ionization); Column at 30° C.; flow rate 1.4 mL/min.

LCMS Method F:

Instrument: Shimadzu Nexera LCMS-2020, photodiode array detector; Column: Synergi 2.5 micron MAX-RP 100 A Mercury (20×4 mm); 3.0 min run time, 5% solvent B from 0 to 0.5 min, 5→95% solvent B from 0.5 to 1.0 min, 95% solvent B from 1.0 to 1.5 min, 95-5% solvent B from 1.5 to 2.0 min, 5% solvent B from 2.0 to 3.0 min. Solvents: Solvent A=0.1% formic acid in water (v/v), solvent B=0.1% formic acid in acetonitrile (v/v). UV detection array 200-400; Mass detection 100-1000 (electrospray ionization); Column at 40° C.; flow rate 2.0 mL/min.

LCMS Method G:

Instrument: API 3000, photodiode array detector; Column: Synergi 2.5 micron MAX-RP 100 A Mercury; 3.0 min run time, 10→20% solvent B from 0.0 to 0.5 min, 20→95% solvent B from 0.5 to 1.5 min, 95% solvent B from 1.5 to 2.0 min, 95→10% solvent B from 2.0 to 2.5 min, 10% solvent B from 2.5 to 3.0 min, 20% solvent B from 3.2 to 4.0 min. Solvents: Solvent A=0.1% formic acid in water (v/v), solvent B=acetonitrile. UV detection array 190-400; Mass detection 100-1000 (electrospray ionization); Column at 30° C.; flow rate 1.4 mL/min.

LCMS Method H:

Instrument: Waters Acquity UPLC, photodiode array detector; Column: SunFire C18 3.5 μm 3.0×30 mm; 2.2 min run time, 5→95% solvent B from 0.0 to 1.7 min, 95% solvent B from 1.7 to 2.0 min, 95→5% solvent B from 2.0 to 2.1 min, 5% solvent B from 2.1 to 2.2 min. Solvents: Solvent A=0.05% TFA in water (v/v), solvent B=acetonitrile. UV detection array 200-400 nm; mass detection 150-1600 (electrospray ionization); column at 40° C.; flow rate 2.0 mL/min.

LCMS Method I:

Column: Kinetex EVO C18 2.1×30 mm, 5 μm; 1.5 min run time, 5→95% solvent B from 0.0 to 0.8 min, 95% solvent B from 0.8 to 1.2 min, 95→5% solvent B from 1.2 to 1.21 min, 5% B from 1.21 to 1.5 min. Solvents: solvent A=0.05% NH₃·H₂O in water (v/v), solvent B=Acetonitrile. Mass detection 100-1000 (electrospray ionization); column at 40° C.; flow rate 1.5 mL/min.

LCMS Method J:

Column: Chromolith Flash RP-18e 25×2 mm; 1.5 min run time, 5% solvent B from 0.0 to 0.01 min, 5→95% solvent B from 0.01 to 0.80 min, 95% solvent B from 0.80 to 1.2 min, 95→5% solvent B from 1.2 to 1.21 min, 5% B from 1.21 to 1.5 min. Solvents: solvent A=0.0375% TFA in water (v/v), solvent B=0.01875% TFA in acetonitrile (v/v). Mass detection 100-1000 (electrospray ionization); column at 50° C.; flow rate 1.5 mL/min.

LCMS Method K:

Instrument: Waters Acquity UPLC, photodiode array detector; Column: AcQuity UPLC BEH C₁₈ 1.7 μm, 2.1×30 mm; 2 min run time, 2% solvent B from 0 to 0.1 min, 2→98% solvent B from 0.1 to 1.8 min, 2% solvent B for 0.2 min. Solvents: Solvent A=5 mM Ammonium Hydroxide in Water, solvent B=5 mM Ammonium Hydroxide in Acetonitrile. Injection volume 2-5 uL; UV detection array 210-400 nm; mass detection 120-1250 (electrospray ionization); column at 50° C.; flow rate 1.0 mL/min.

### Synthesis of Intermediates and Examples

### Intermediate 1

A racemic mixture of:
(3aS,5S,6aR)-5-phenoxyhexahydrocyclopenta[c]pyrrol-3a(1H)-ol
(3aR,5R,6aS)-5-phenoxyhexahydrocyclopenta[c]pyrrol-3a(1H)-ol

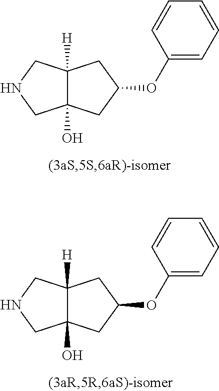 [SMILES: O[C@]12CNC[C@H]1C[C@H](Oc1ccccc1)C2 ; O[C@@]12CNC[C@@H]1C[C@@H](Oc1ccccc1)C2]

### Step 1: Benzyl prop-2-yn-1-ylcarbamate

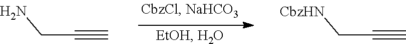

Benzyl chloroformate (273 g, 1.60 mol) was added dropwise to a stirred solution of propargylamine (80 g, 1.45 mol) and NaHCO₃ (243.6 g, 2.9 mol) in ethanol/water (2.4 L, 1:1, v/v) at 0° C. After stirring for 2 h at 0° C. and 12 h at 25° C., the mixture was diluted with water (1.0 L) and extracted with MTBE (1.0 L). The phases were separated and the aqueous layer was extracted with MTBE (500 mL×2). The combined organic layers were dried over anhydrous Na₂SO₄, filtered and evaporated to give the title intermediate (280 g, crude) as a yellow solid which was used without purification. ¹H NMR (400 MHz, CDCl₃) δ 7.38-7.32 (m, 5H), 5.24-5.08 (m, 3H), 4.05-3.93 (m, 2H), 2.26 (s, 1H).

### Step 2: Benzyl allyl(prop-2-yn-1-yl)carbamate

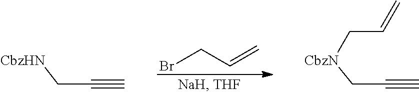

NaH (60% in mineral oil, 39 g, 0.98 mol) was added to a solution of benzyl prop-2-yn-1-ylcarbamate (155 g, 0.817 mol) and allyl bromide (149 g, 1.23 mol) in THF (2.0 L) at 0° C. and the reaction was stirred for 2 h at 25° C. The mixture was quenched with saturated aq. NH₄Cl (500 mL) and the aqueous layer was extracted with EtOAc (3×500 mL). The combined organic layers were dried over anhydrous Na₂SO₄ and concentrated. The crude material was purified by FCC (10% EtOAc:PE) to give the title intermediate (135 g) as a colorless oil.

¹H NMR (400 MHz, CDCl₃) δ 7.44-7.31 (m, 5H), 5.87-5.74 (m, 1H), 5.29-5.15 (m, 4H), 4.17-3.96 (m, 4H), 2.23 (s, 1H).

### Step 3: (±)-Benzyl 5-oxo-3,3a,4,5-tetrahydrocyclopenta[c]pyrrole-2(1H)-carboxylate

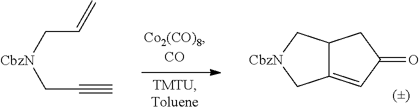 [SMILES: C#CCN(CC=C)C(=O)OCc1ccccc1 ; O=C1C=C2CN(C(=O)OCc3ccccc3)CC2C1]

To a solution of benzyl allyl(prop-2-yn-1-yl)carbamate (20 g, 89.6 mmol) and N,N,N,N-tetramethylthiourea (5.89 g, 44.5 mmol) in toluene (1.0 L) was added CO₂(CO)₈ (7.6 g, 22.4 mmol) at 25° C. under 1 atm CO pressure. The solution was heated to 80° C. and stirred for 3 h. The reaction mixture was cooled to RT, filtered through a pad of Celite and concentrated. The crude material was purified by FCC (15-50% EtOAc:PE) to provide the title intermediate (12 g) as a colorless oil.

¹H NMR (400 MHz, CDCl₃) δ 7.38-7.33 (m, 5H), 6.11-6.07 (m, 1H), 5.21-5.14 (m, 2H), 4.36-4.28 (m, 2H), 4.18-4.11 (m, 1H), 3.28-3.26 (m, 1H), 2.97-2.92 (m, 1H), 2.68-2.64 (m, 1H), 2.23-2.19 (m, 1H).

### Step 4: A Racemic Mixture of: Benzyl (3aS,6aR)-3a-hydroxy-5-oxohexahydrocyclopenta[c]pyrrole-2(1H)-carboxylate Benzyl (3aR,6aS)-3a-hydroxy-5-oxohexahydrocyclopenta[c]pyrrole-2(1H)-carboxylate

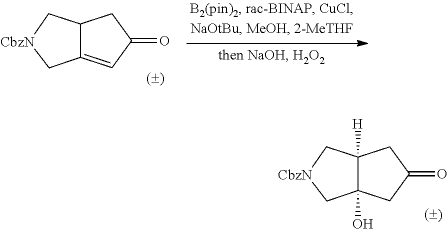 [SMILES: O=C1C=C2CN(C(=O)OCc3ccccc3)CC2C1 ; O=C1C[C@@H]2CN(C(=O)OCc3ccccc3)C[C@]2(O)C1]

2-methyltetrahydrofuran (125 mL) was purged with nitrogen for 10 minutes, then CuCl (485 mg, 4.9 mmol) and rac-BINAP (3.03 g, 4.9 mmol) were added. After 5 minutes NaOt-Bu (470 mg, 4.9 mmol) and bis(pinacolato)diboron (30 g, 117 mmol) were added and the reaction was purged with nitrogen for another 15 minutes. A solution of (±)-benzyl 5-oxo-3,3a,4,5-tetrahydrocyclopenta[c]pyrrole-2(1H)-carboxylate (25 g, 97 mmol) in 2-methyltetrahydrofuran (125 mL) was added and the reaction was stirred under nitrogen at RT for 2 h. The reaction was cooled to 10° C. and MeOH (6.25 g, 7.89 mL, 194 mmol) was added. This was stirred for 10 min, then warmed to RT for 30 min, then cooled again to 10° C. NaOH (4.66 g, 117 mmol) was added followed by 30% aq. H₂O₂ (33 g, 99 mL, 292 mmol) dropwise, and this was stirred for 50 min. This was diluted with water (150 mL) and extracted with EtOAc (3×100 mL). The combined organic layers were washed with saturated aq. sodium thiosulfate (100 mL), dried with Na₂SO₄, filtered and concentrated. The crude material was purified by FCC (0-100% EtOAc:PE) to provide the title intermediate (20 g, 90% purity) as a light yellow oil.

¹H NMR (400 MHz, DMSO-d₆) δ 7.43-7.24 (m, 5H), 5.55 (s, 1H), 5.12-4.99 (m, 2H), 3.79-3.65 (m, 1H), 3.53-3.38 (m, 2H), 3.22-3.11 (m, 1H), 2.70-2.62 (m, 1H), 2.58-2.52 (m, 1H), 2.34-2.29 (m, 1H), 2.17-2.06 (m, 1H). 1H under solvent peak.

### Step 5: A Racemic Mixture of: Benzyl (3aS,5R,6aR)-3a,5-dihydroxyhexahydrocyclopenta[c]pyrrole-2(1H)-carboxylate Benzyl (3aR,5S,6aS)-3a,5-dihydroxyhexahydrocyclopenta[c]pyrrole-2(1H)-carboxylate

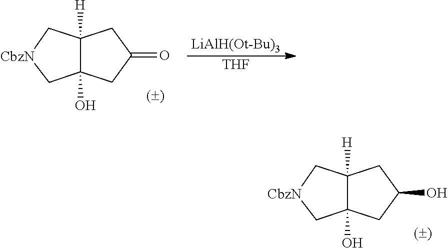 [SMILES: O=C1C[C@@H]2CN(C(=O)OCc3ccccc3)C[C@]2(O)C1 ; O=C(OCc1ccccc1)N1C[C@H]2C[C@@H](O)C[C@@]2(O)C1 ; "LiAlH(Ot-Bu)3" ; "THF"]

To a solution of a racemic mixture of benzyl (3aS,6aR)-3a-hydroxy-5-oxohexahydrocyclopenta[c]pyrrole-2(1H)-carboxylate and benzyl (3aR,6aS)-3a-hydroxy-5-oxohexahydrocyclopenta[c]pyrrole-2(1H)-carboxylate (20 g, 62.48 mmol, 90% purity) in THF (200 mL) was added dropwise a solution of LiAlH(Ot-Bu)₃ (124.9 mL, 124.9 mmol, 1.0 M in THF) at 0° C. The reaction was warmed to 25° C. and stirred for 2 h. The reaction mixture was added dropwise to a saturated solution of NH₄Cl (100 mL) at 0° C. The mixture was extracted with EtOAc (2×100 mL). The combined organic layers were washed with saturated brine (100 mL). The organic layer was dried over Na₂SO₄ and concentrated. The crude material was purified by FCC (0-15% MeOH:DCM) to provide the title intermediate (16 g) as a colorless oil.

LCMS: Rt 0.56 min; MS m/z 278.1 [M+H]⁺; Method J.

¹H NMR (400 MHz, DMSO-d₆) δ 7.39-7.29 (m, 5H), 5.06-5.01 (m, 3H), 4.67-4.65 (m, 1H), 4.28-4.19 (m, 1H), 3.65-3.52 (m, 2H), 3.38-3.34 (m, 1H), 3.27-3.17 (m, 1H), 2.32-2.13 (m, 2H), 2.05-1.92 (m, 1H), 1.73-1.64 (m, 1H), 1.29-1.16 (m, 1H).

### Step 6: A Racemic Mixture of: Benzyl (3aS,5S,6aR)-3a-hydroxy-5-phenoxyhexahydrocyclopenta[c]pyrrole-2(1H)-carboxylate Benzyl (3aR,5R,6aS)-3a-hydroxy-5-phenoxyhexahydrocyclopenta[c]pyrrole-2(1H)-carboxylate

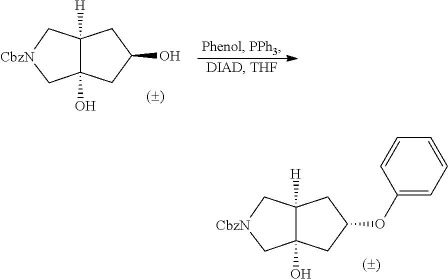

A dried reaction flask was charged with triphenylphosphine (12.58 g, 48.0 mmol), anhydrous THF (100 mL) and phenol (4.84 g, 51.4 mmol) with stirring under nitrogen at ambient temperature. A racemic mixture of benzyl (3aS,5R,6aR)-3a,5-dihydroxyhexahydrocyclopenta[c]pyrrole-2(1H)-carboxylate and benzyl (3aR,5S,6aS)-3a,5-dihydroxyhexahydrocyclopenta[c]pyrrole-2(1H)-carboxylate (9.5 g, 34.3 mmol) in anhydrous THF (10.5 mL) was added and the solution was cooled in an ice bath. A solution of DIAD (9.32 mL, 48.0 mmol) in anhydrous THF (50 mL) was added dropwise over 15-20 minutes with vigorous stirring, and a light yellow color persisted upon complete addition. The maximum internal temperature reached about 14° C. during the addition, and the reaction was aged in the bath for 45 minutes. The reaction was quenched with water (50 mL), and the mixture was stirred for about 30 minutes. The mixture was diluted with EtOAc (100 mL), and the organic layer was washed a second time with water (50 mL). The combined aqueous washes were back-extracted with EtOAc (100 mL), and the combined organic extracts were washed with saturated brine (2×100 mL), and then dried over Na₂SO₄, filtered and concentrated to a yellow oil. The residue was triturated with Et₂O (100 mL), resulting in an off-white precipitate, and the mixture was stirred in an ice/water bath while heptanes (50 mL) was added dropwise with vigorous stirring. The precipitate was collected and washed with 1:2 heptanes/Et₂O. The light yellow solid product was slurried again with Et₂O first by rotation on the rotovap at 35° C., and then with stirring at room temperature overnight. The slurry was filtered and all the filtrate was combined. The filtrate/wash was concentrated to dryness and the yellow oil was treated with Et₂O/heptane (2:1) and purified by FCC (10-60% EtOAc:Hexane) to give the title intermediate (11.46 g).

LCMS: Rt 2.29 min; MS m/z 354.4 [M+H]⁺; Method B.

¹H NMR (400 MHz, CDCl₃) δ 7.38-7.28 (m, 7H), 7.01-6.96 (m, 1H), 6.88-6.85 (m, 2H), 5.14 (s, 2H), 4.95-4.92 (m, 1H), 3.81-3.78 (m, 2H), 3.50-3.46 (m, 1H), 3.30-3.25 (m, 1H), 2.76-2.72 (m, 2H), 2.47-2.41 (m, 1H), 2.32-2.27 (m, 1H), 2.18-2.10 (m, 1H), 1.75 (m, 1H).

### Step 7: A Racemic Mixture of: (3aS,5S,6aR)-5-phenoxyhexahydrocyclopenta[c]pyrrol-3a(1H)-ol (3aR,5R,6aS)-5-phenoxyhexahydrocyclopenta[c]pyrrol-3a(1H)-ol

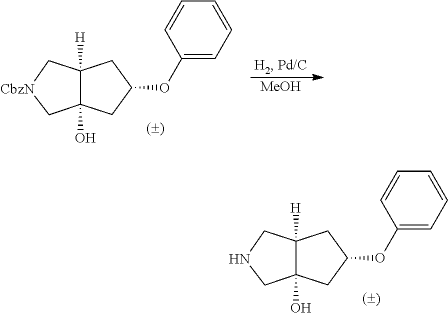 [SMILES: "MeOH" ; O=C(OCc1ccccc1)N1C[C@H]2C[C@H](Oc3ccccc3)C[C@@]2(O)C1 ; "H2, Pd/C" ; O[C@]12CNC[C@H]1C[C@H](Oc1ccccc1)C2]

The flask containing benzyl (3aS,5S,6aR)-3a-hydroxy-5-phenoxyhexahydrocyclopenta[c]pyrrole-2(1H)-carboxylate and benzyl (3aR,5R,6aS)-3a-hydroxy-5-phenoxyhexahydrocyclopenta[c]pyrrole-2(1H)-carboxylate (11.46 g, 32.4 mmol) was equipped with a magnetic stirbar and purged with nitrogen. To the flask was added anhydrous MeOH (200 mL) with stirring at ambient temperature. The flask was purged of oxygen by performing two vacuum-to-N₂ cycles on the manifold, and then Pd/C (10% Pd loading, Degussa wet-type, 0.724 g, 6.80 mmol) was charged with stirring. The flask was stoppered with a rubber septum and vacuum purged twice cycling from nitrogen to vacuum. The H₂ balloon was affixed to a long syringe needle extending below the level of the liquid, and the vacuum was broken by opening the H₂ balloon to the evacuated flask using a plastic Luer stopcock. The reaction was vigorously stirred at room temperature for 2 h. A nitrogen inlet was placed into the flask and the flask was purged for 15 min. The reaction mixture was filtered through a pad of Celite, washing through with DCM. The filtrate was concentrated to yield the title intermediate as a white solid (6.3 g), which was used in the next step without purification.

LCMS: Rt 0.85 min; MS m/z 220.3 [M+H]⁺; Method B.

¹H NMR (400 MHz, Methanol-d₄) δ 7.31-7.19 (m, 2H), 6.97-6.82 (m, 3H), 3.24 (dd, J=11.6, 7.7 Hz, 1H), 2.94-2.81 (m, 2H), 2.66-2.48 (m, 2H), 2.31-2.15 (m, 2H), 2.09 (ddd, J=13.9, 4.7, 1.8 Hz, 1H), 1.81-1.69 (m, 1H). 1H under solvent peak.

### Intermediate 2

### (3aS,5S,6aR)-5-phenoxyhexahydrocyclopenta[c]pyrrol-3a(1H)-ol

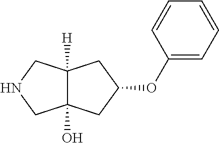 [SMILES: O[C@]12CNC[C@H]1C[C@H](Oc1ccccc1)C2]

### Step 1: Benzyl (3aS,5R,6aR)-3a,5-dihydroxyhexahydrocyclopenta[c]pyrrole-2(1H)-carboxylate

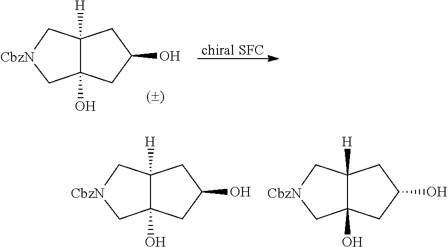 [SMILES: O=C(OCc1ccccc1)N1C[C@H]2C[C@@H](O)C[C@@]2(O)C1 ; "chiral SFC" ; O=C(OCc1ccccc1)N1C[C@H]2C[C@@H](O)C[C@@]2(O)C1 ; O=C(OCc1ccccc1)N1C[C@@H]2C[C@H](O)C[C@]2(O)C1]

The racemic mixture of benzyl (3aS,5R,6aR)-3a,5-dihydroxyhexahydrocyclopenta[c]pyrrole-2(1H)-carboxylate and benzyl (3aR,5S,6aS)-3a,5-dihydroxyhexahydrocyclopenta[c]pyrrole-2(1H)-carboxylate (from step 5 of Intermediate 1) (450 mg) was separated by chiral SFC using the condition below to provide benzyl (3aS,5R,6aR)-3a,5-dihydroxyhexahydrocyclopenta[c]pyrrole-2(1H)-carboxylate (190 mg, peak 1) as a colorless oil and benzyl (3aR,5S,6aS)-3a,5-dihydroxyhexahydrocyclopenta[c]pyrrole-2(1H)-carboxylate (220 mg, peak 2) as a colorless oil.

Column: Chiralpak AD (250 mm×30 mm, 10 μm), Flow rate: 70 g/min

Mobile phase: CO₂ (A), MeOH with 0.1% NH₄OH (B), Isocratic 60:40 (A:B)

Peak 1:

Chiral SFC: Rt 1.58 min (Column: Chiralpak AD-3 50×4.6 mm I.D., 3 μm, Flow rate: 3 mL/min, Mobile phase: CO₂ (A), MeOH with 0.05% DEA (B), Gradient elution: 5-40% B). ¹H NMR (400 MHz, CDCl₃) δ 7.38-7.29 (m, 5H), 5.16 (s, 2H), 4.56-4.52 (m, 1H), 3.82-3.76 (m, 2H), 3.56-3.53 (m, 1H), 3.44-3.41 (m, 1H), 2.48-2.39 (m, 2H), 2.24-2.18 (m, 1H), 1.99-1.94 (m, 1H), 1.81 (br s, 1H), 1.65 (br s, 1H), 1.54-1.41 (m, 1H).

Peak 2:

Chiral SFC: Rt 2.04 min (Column: Chiralpak AD-3 50×4.6 mm I.D., 3 μm, Flow rate: 3 mL/min, Mobile phase: CO₂ (A), MeOH with 0.05% DEA (B), Gradient elution: 5-40% B). ¹H NMR (400 MHz, CDCl₃) δ 7.38-7.31 (m, 5H), 5.14 (s, 2H), 4.56-4.51 (m, 1H), 3.82-3.76 (m, 2H), 3.56-3.52 (m, 1H), 3.44-3.41 (m, 1H), 2.47-2.39 (m, 2H), 2.24-2.18 (m, 1H), 1.99-1.94 (m, 1H), 1.82 (br s, 1H), 1.65 (br s, 1H), 1.51-1.41 (m, 1H).

### Step 2: Benzyl (3aS,5S,6aR)-3a-hydroxy-5-phenoxyhexahydrocyclopenta[c]pyrrole-2(1H)-carboxylate

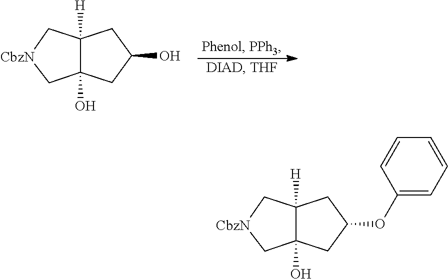

Starting with benzyl (3aS,5R,6aR)-3a,5-dihydroxyhexahydrocyclopenta[c]pyrrole-2(1H)-carboxylate (peak 1 from the previous step), and following the procedure used in step 6 of Intermediate 1, provided the title intermediate.

LCMS: Rt 0.84 min; MS m/z 354.2 [M+H]⁺; Method J.

¹H NMR (400 MHz, CDCl₃) δ 7.38-7.28 (m, 7H), 7.01-6.96 (m, 1H), 6.88-6.85 (m, 2H), 5.14 (s, 2H), 4.95-4.92 (m, 1H), 3.81-3.78 (m, 2H), 3.50-3.46 (m, 1H), 3.30-3.25 (m, 1H), 2.76-2.72 (m, 2H), 2.47-2.41 (m, 1H), 2.32-2.27 (m, 1H), 2.18-2.10 (m, 1H), 1.75 (m, 1H).

### Step 3: (3aS,5S,6aR)-5-phenoxyhexahydrocyclopenta[c]pyrrol-3a(1H)-ol

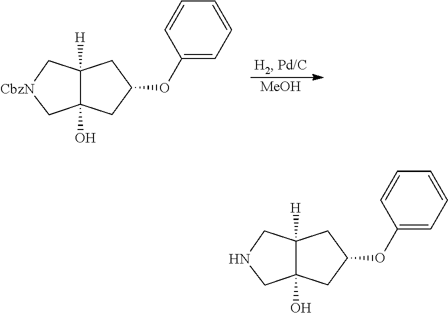 [SMILES: "MeOH" ; O=C(OCc1ccccc1)N1C[C@H]2C[C@H](Oc3ccccc3)C[C@@]2(O)C1 ; "H2, Pd/C" ; O[C@]12CNC[C@H]1C[C@H](Oc1ccccc1)C2]

Starting with benzyl (3aS,5S,6aR)-3a-hydroxy-5-phenoxyhexahydrocyclopenta[c]pyrrole-2(1H)-carboxylate, and following the procedure used in step 7 of Intermediate 1, provided the title intermediate.

LCMS: Rt 0.86 min; MS m/z 220.0 [M+H]⁺; Method I.

¹H NMR (400 MHz, DMSO-d₆) δ 7.33-7.21 (m, 2H), 6.94-6.84 (m, 3H), 4.88-4.66 (m, 2H), 3.06-3.01 (m, 1H), 2.72-2.65 (m, 2H), 2.53-2.51 (m, 1H), 2.46-2.42 (m, 1H), 2.30-2.14 (m, 2H), 2.04-1.94 (m, 1H), 1.92-1.86 (m, 1H), 1.80-1.71 (m, 1H).

### Intermediate 3

A racemic mixture of:
(3aS,5S,6aR)-5-(2-fluorophenoxy)hexahydrocyclopenta[c]pyrrol-3a(1H)-ol
(3aR,5R,6aS)-5-(2-fluorophenoxy)hexahydrocyclopenta[c]pyrrol-3a(1H)-ol

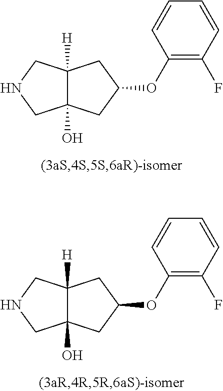 [SMILES: O[C@]12CNC[C@H]1C[C@H](Oc1ccccc1F)C2 ; O[C@@]12CNC[C@@H]1C[C@@H](Oc1ccccc1F)C2]

This was synthesized in a similar manner as Intermediate 1, using 2-fluorophenol in step 6.

LCMS: Rt 0.66 min; MS m/z 238.3 [M+H]⁺; Method B.

¹H NMR (400 MHz, DMSO-d₆) δ 7.26-7.14 (m, 2H), 7.14-7.02 (m, 1H), 6.96-6.87 (m, 1H), 4.88-4.79 (m, 1H), 4.73 (br s, 1H), 3.07-3.01 (m, 1H), 2.73-2.66 (m, 2H), 2.47-2.43 (m, 1H), 2.36-2.26 (m, 1H), 2.23-2.17 (m, 1H), 2.08-1.99 (m, 1H), 1.96-1.91 (m, 1H), 1.80-1.73 (m, 1H). 1H under solvent peak.

### Intermediate 4

### (3aS,5S,6aR)-5-(2-fluorophenoxy)hexahydrocyclopenta[c]pyrrol-3a(1H)-ol

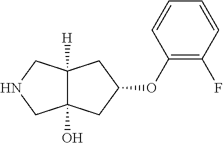 [SMILES: O[C@]12CNC[C@H]1C[C@H](Oc1ccccc1F)C2]

This was synthesized in a similar manner as Intermediate 2, using 2-fluorophenol in step 2.

LCMS: Rt 0.87 min; MS m/z 238.3 [M+H]⁺; Method I.

¹H NMR (400 MHz, CDCl₃) δ 7.21-7.15 (m, 2H), 7.12-7.08 (m, 1H), 6.95-6.89 (m, 1H), 4.85-4.79 (m, 1H), 4.74 (br s, 1H), 3.07-3.01 (m, 1H), 2.73-2.66 (m, 2H), 2.47-2.43 (m, 1H), 2.36-2.25 (m, 1H), 2.23-2.17 (m, 1H), 2.08-1.99 (m, 1H), 1.97-1.91 (m, 1H), 1.79-1.73 (m, 1H). 1H under solvent peak.

### Intermediates 5 and 6

### (3aS,4S,5S,6aR)-5-phenoxyhexahydrocyclopenta[c]pyrrole-3a,4(1H)-diol

### (3aR,4R,5R,6aS)-5-phenoxyhexahydrocyclopenta[c]pyrrole-3a,4(1H)-diol

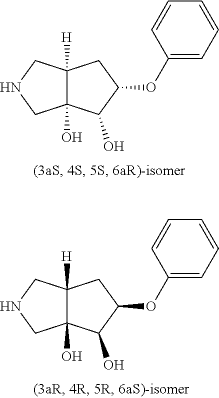 [SMILES: O[C@H]1[C@@H](Oc2ccccc2)C[C@@H]2CNC[C@@]21O ; O[C@@H]1[C@H](Oc2ccccc2)C[C@H]2CNC[C@]21O]

### Step 1: A Racemic Mixture of: Benzyl (3aS,5R)-5-hydroxy-3,3a,4,5-tetrahydrocyclopenta[c]pyrrole-2(1H)-carboxylate Benzyl (3aR,5S)-5-hydroxy-3,3a,4,5-tetrahydrocyclopenta[c]pyrrole-2(1H)-carboxylate

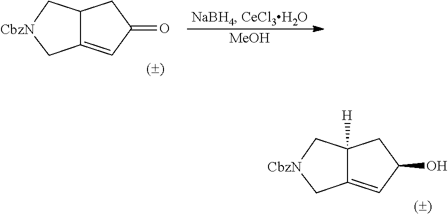

To a stirred solution of (±)-benzyl 5-oxo-3,3a,4,5-tetrahydrocyclopenta[c]pyrrole-2(1H)-carboxylate (from step 3 of Intermediate 1) (2.0 g, 7.8 mmol) in methanol (500 mL) was added CeCl₃·H₂O (5.7 g, 23.3 mmol) followed by NaBH₄ (0.35 g, 9.36 mmol) at −70° C. The reaction mixture was stirred at RT for 4 h. The reaction mixture was concentrated, and the material was dissolved in EtOAc and washed with water. The organic layer was dried over Na₂SO₄, concentrated, and purified by FCC (60% EtOAc:Hexane) to provide the title intermediate (1.6 g).

LCMS: Rt 0.50 min; MS m/z 260.2 [M+H]⁺; Method D.

¹H NMR (400 MHz, CDCl₃) δ 7.37-7.29 (m, 5H), 5.59 (d, J=16 Hz, 1H), 5.14 (m, 3H), 4.04 (dd, J=16.0, 6.0 Hz, 1H), 3.97-3.88 (m, 2H), 3.08-2.96 (m, 1H), 2.88 (t, J=9.6 Hz, 1H), 2.72-2.61 (m, 1H), 1.83 (t, J=10.0 Hz, 1H), 1.40-1.28 (m, 1H).

### Step 2: A Racemic Mixture of: Benzyl (3aS,5S)-5-phenoxy-3,3a,4,5-tetrahydrocyclopenta[c]pyrrole-2(1H)-carboxylate Benzyl (3aR,5R)-5-phenoxy-3,3a,4,5-tetrahydrocyclopenta[c]pyrrole-2(1H)-carboxylate

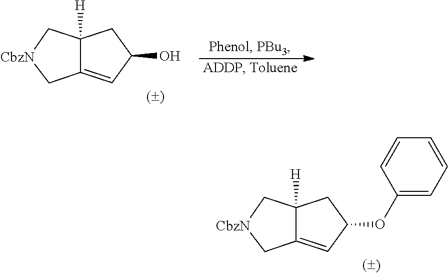

To a solution of the racemate of benzyl (3aS,5R)-5-hydroxy-3,3a,4,5-tetrahydrocyclopenta[c]pyrrole-2(1H)-carboxylate and benzyl (3aR,5S)-5-hydroxy-3,3a,4,5-tetrahydrocyclopenta[c]pyrrole-2(1H)-carboxylate (6.0 g, 23.1 mmol), phenol (2.6 g, 27.7 mmol) and 1,1′-(azodicarbonyl)dipiperidine (11.6 g, 46.2 mmol) in toluene (500 mL) was added tributylphosphine (14 g, 69.3 mmol) at RT and the reaction mixture was stirred at 100° C. for 16 h. The reaction mixture was cooled to RT, filtered and the filtrate was concentrated. The crude material was purified by FCC (10% EtOAc:Hexane) to provide the title intermediate (3.5 g).

¹H NMR (400 MHz, CDCl₃) δ 7.39-7.26 (m, 7H), 6.96-6.92 (m, 1H), 6.89 (d, J=8 Hz, 2H), 5.87 (d, J=14.8 Hz, 1H), 5.46 (dd, J=3.6, 2.4 Hz, 1H), 5.19-5.12 (m, 2H), 4.08-3.95 (m, 3H), 3.60-3.50 (m, 1H), 2.80 (dt, 10.4, 1.2 Hz, 1H), 2.39-2.30 (m, 1H), 1.90-1.83 (m, 1H).

### Step 3: A Racemic Mixture of: Benzyl (3aS,4S,5S,6aR)-3a,4-dihydroxy-5-phenoxyhexahydrocyclopenta[c]pyrrole-2(1H)-carboxylate Benzyl (3aR,4R,5R,6aS)-3a,4-dihydroxy-5-phenoxyhexahydrocyclopenta[c]pyrrole-2(1H)-carboxylate

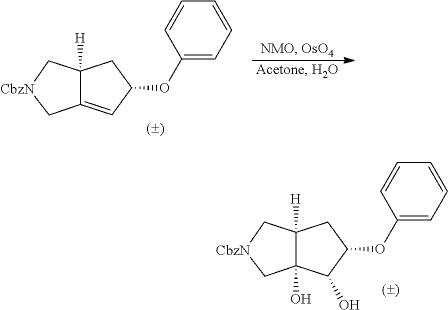

To a solution of the racemate of benzyl (3aS,5S)-5-phenoxy-3,3a,4,5-tetrahydrocyclopenta[c]pyrrole-2(1H)-carboxylate and benzyl (3aR,5R)-5-phenoxy-3,3a,4,5-tetrahydrocyclopenta[c]pyrrole-2(1H)-carboxylate (2.5 g, 7.4 mmol) and N-methyl morpholine N-oxide monohydrate (17 g, 126.5 mmol) in acetone (200 mL) and water (200 mL) was added a solution of OSO₄ (96 mg, 0.37 mmol) in t-BuOH (20 mL) at RT and the reaction mixture was stirred for 16 h. The reaction mixture was extracted with ethyl acetate, dried over Na₂SO₄, concentrated, and purified by FCC (50% EtOAc:Hexane) to provide the title intermediate (2.5 g).

LCMS: Rt 1.40 min; MS m/z 370.3 [M+H]⁺; Method D.

### Step 4: Chiral Separation of: Benzyl (3aS,4S,5S,6aR)-3a,4-dihydroxy-5-phenoxyhexahydrocyclopenta[c]pyrrole-2(1H)-carboxylate Benzyl (3aR,4R,5R,6aS)-3a,4-dihydroxy-5-phenoxyhexahydrocyclopenta[c]pyrrole-2(1H)-carboxylate

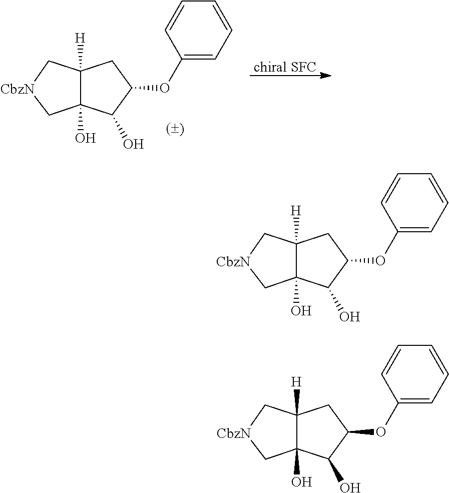 [SMILES: O=C(OCc1ccccc1)N1C[C@H]2C[C@H](Oc3ccccc3)[C@H](O)[C@@]2(O)C1 ; "chiral SFC" ; O=C(OCc1ccccc1)N1C[C@H]2C[C@H](Oc3ccccc3)[C@H](O)[C@@]2(O)C1 ; O=C(OCc1ccccc1)N1C[C@@H]2C[C@@H](Oc3ccccc3)[C@@H](O)[C@]2(O)C1]

The racemic mixture of benzyl (3aS,4S,5S,6aR)-3a,4-dihydroxy-5-phenoxyhexahydrocyclopenta[c]pyrrole-2(1H)-carboxylate and benzyl (3aR,4R,5R,6aS)-3a,4-dihydroxy-5-phenoxyhexahydrocyclopenta[c]pyrrole-2(1H)-carboxylate (2.5 g) was separated by chiral SFC using the method below to provide benzyl (3aS,4S,5S,6aR)-3a,4-dihydroxy-5-phenoxyhexahydrocyclopenta[c]pyrrole-2(1H)-carboxylate (chiral SFC Rt 7.23 min, 1.2 g) and benzyl (3aR,4R,5R,6aS)-3a,4-dihydroxy-5-phenoxyhexahydrocyclopenta[c]pyrrole-2(1H)-carboxylate (chiral SFC Rt 5.86 min, 1.2 g).

Column: Chiralpak IG (10 mm×250 mm, 5 micron), Flow: 13 mL/min

Mobile phase: CO₂ (A), EtOH:IPA, 1:1 (B), Isocratic 70:30 (A:B)

### Step 5: (3aS,4S,5S,6aR)-5-phenoxyhexahydrocyclopenta[c]pyrrole-3a,4(1H)-diol (Intermediate 5)

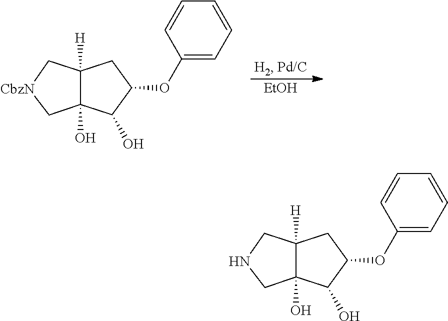 [SMILES: "EtOH" ; O=C(OCc1ccccc1)N1C[C@H]2C[C@H](Oc3ccccc3)[C@H](O)[C@@]2(O)C1 ; "H2, Pd/C" ; O[C@H]1[C@@H](Oc2ccccc2)C[C@@H]2CNC[C@@]21O]

A solution of benzyl (3aS,4S,5S,6aR)-3a,4-dihydroxy-5-phenoxyhexahydrocyclopenta[c]pyrrole-2(1H)-carboxylate (chiral SFC Rt 7.23 min from step 4) (1.2 g, 3.24 mmol) in EtOH (100 mL) was shaken with 10% Pd on carbon (120 mg) under H₂ (balloon pressure) for 6 h. The reaction mixture was filtered through Celite and concentrated to provide the title intermediate (750 mg) which was used without further purification.

LCMS: Rt 0.55 min; MS m/z 236.0 [M+H]⁺; Method E.

¹H NMR (400 MHz, Methanol-d₄) δ 7.27-7.23 (m, 2H), 7.01-6.99 (m, 2H), 6.92 (t, J=7.2 Hz, 1H), 4.78-4.73 (m, 1H), 3.94 (d, J=3.6 Hz, 1H), 3.23-3.19 (m, 1H), 2.97 (d, J=12.0 Hz, 1H), 2.86 (d, J=12.0 Hz, 1H), 2.70-2.65 (m, 1H), 2.54-2.49 (m, 1H), 2.30-2.23 (m, 1H), 1.60-1.55 (m, 1H).

### Step 6: (3aR,4R,5R,6aS)-5-phenoxyhexahydrocyclopenta[c]pyrrole-3a,4(1H)-diol (Intermediate 6)

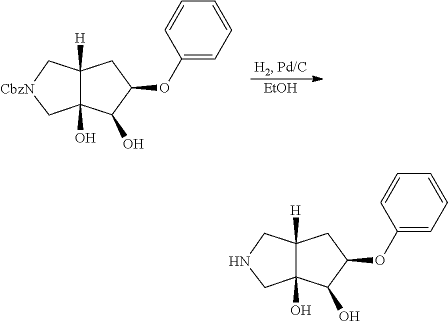 [SMILES: "EtOH" ; O=C(OCc1ccccc1)N1C[C@@H]2C[C@@H](Oc3ccccc3)[C@@H](O)[C@]2(O)C1 ; "H2, Pd/C" ; O[C@@H]1[C@H](Oc2ccccc2)C[C@H]2CNC[C@]21O]

Using the same method as step 5, starting from benzyl (3aR,4R,5R,6aS)-3a,4-dihydroxy-5-phenoxyhexahydrocyclopenta[c]pyrrole-2(1H)-carboxylate (chiral SFC Rt 5.86 min from step 4) (1.2 g, 3.24 mmol), provided the title intermediate (750 mg).

LCMS: Rt 0.55 min; MS m/z 236.0 [M+H]⁺; Method E.

¹H NMR (400 MHz, Methanol-d₄) δ 7.27-7.23 (m, 2H), 7.01-6.99 (m, 2H), 6.92 (t, J=7.2 Hz, 1H), 4.78-4.73 (m, 1H), 3.93 (d, J=4.0 Hz, 1H), 3.20-3.15 (m, 1H), 2.94 (d, J=12.4 Hz, 1H), 2.82 (d, J=12.0 Hz, 1H), 2.66-2.63 (m, 1H), 2.52-2.46 (m, 1H), 2.30-2.23 (m, 1H), 1.60-1.52 (m, 1H).

### Intermediate 7

A racemic mixture of:
(3aS,4S,5S,6aR)-5-(2-fluorophenoxy)hexahydrocyclopenta[c]pyrrole-3a,4(1H)-diol
(3aR,4R,5R,6aS)-5-(2-fluorophenoxy)hexahydrocyclopenta[c]pyrrole-3a,4(1H)-diol

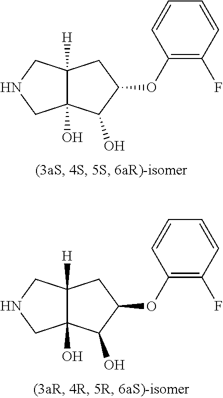 [SMILES: O[C@H]1[C@@H](Oc2ccccc2F)C[C@@H]2CNC[C@@]21O ; O[C@@H]1[C@H](Oc2ccccc2F)C[C@H]2CNC[C@]21O]

### Steps 1-3: A Racemic Mixture of: Benzyl (3aS,4S,5S,6aR)-5-(2-fluorophenoxy)-3a,4-dihydroxyhexahydrocyclopenta[c]pyrrole-2(1H)-carboxylate Benzyl (3aR,4R,5R,6aS)-5-(2-fluorophenoxy)-3a,4-dihydroxyhexahydrocyclopenta[c]pyrrole-2(1H)-carboxylate

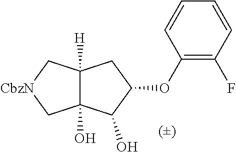 [SMILES: O=C(OCc1ccccc1)N1C[C@H]2C[C@H](Oc3ccccc3F)[C@H](O)[C@@]2(O)C1]

The title intermediate was synthesized using the same methods as steps 1-3 of Intermediates 5 and 6, using 2-fluorophenol in step 2 instead of phenol.

LCMS: Rt 1.44 min; MS m/z 388.0 [M+H]⁺; Method D.

### Step 4: A Racemic Mixture of: (3aS,4S,5S,6aR)-5-(2-fluorophenoxy)hexahydrocyclopenta[c]pyrrole-3a,4(1H)-diol (3aR,4R,5R,6aS)-5-(2-fluorophenoxy)hexahydrocyclopenta[c]pyrrole-3a,4(1H)-diol

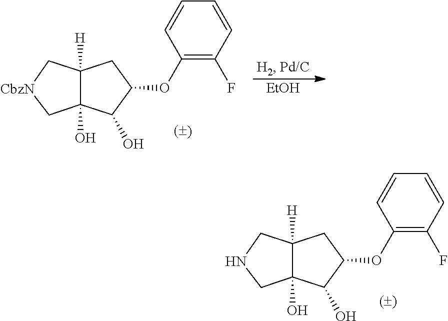 [SMILES: "EtOH" ; O=C(OCc1ccccc1)N1C[C@H]2C[C@H](Oc3ccccc3F)[C@H](O)[C@@]2(O)C1 ; "H2, Pd/C" ; O[C@H]1[C@@H](Oc2ccccc2F)C[C@@H]2CNC[C@@]21O]

Using the same method as step 5 of Intermediate 5, starting from a racemic mixture of benzyl (3aS,4S,5S,6aR)-5-(2-fluorophenoxy)-3a,4-dihydroxyhexahydrocyclopenta[c]pyrrole-2(1H)-carboxylate and benzyl (3aR,4R,5R,6aS)-5-(2-fluorophenoxy)-3a,4-dihydroxyhexahydrocyclopenta[c]pyrrole-2(1H)-carboxylate (200 mg), provided the title intermediate (130 mg).

LCMS: Rt 0.11 min; MS m/z 253.9 [M+H]⁺; Method D.

### Intermediate 8

### (3aS,4S,5S,6aR)-5-(2-fluorophenoxy)hexahydrocyclopenta[c]pyrrole-3a,4(1H)-diol

### Step 1: Chiral Separation of

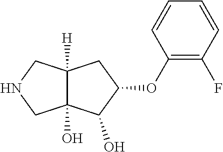 [SMILES: O[C@H]1[C@@H](Oc2ccccc2F)C[C@@H]2CNC[C@@]21O] [0423] Benzyl (3aS,4S,5S,6aR)-5-(2-fluorophenoxy)-3a,4-dihydroxyhexahydrocyclopenta[c]pyrrole-2(1H)-carboxylate
Benzyl (3aR,4R,5R,6aS)-5-(2-fluorophenoxy)-3a,4-dihydroxyhexahydrocyclopenta[c]pyrrole-2(1H)-carboxylate

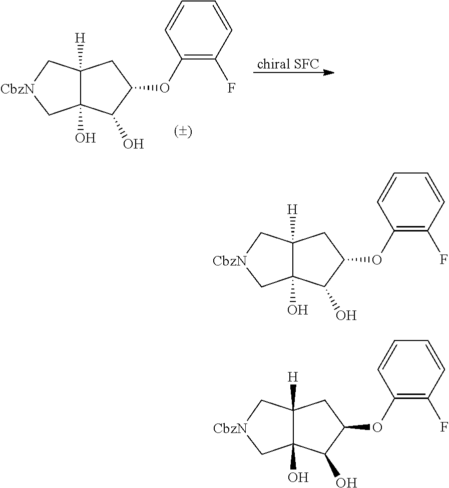 [SMILES: O=C(OCc1ccccc1)N1C[C@H]2C[C@H](Oc3ccccc3F)[C@H](O)[C@@]2(O)C1 ; "chiral SFC" ; O=C(OCc1ccccc1)N1C[C@H]2C[C@H](Oc3ccccc3F)[C@H](O)[C@@]2(O)C1 ; O=C(OCc1ccccc1)N1C[C@@H]2C[C@@H](Oc3ccccc3F)[C@@H](O)[C@]2(O)C1]

The racemic mixture of benzyl (3aS,4S,5S,6aR)-5-(2-fluorophenoxy)-3a,4-dihydroxyhexahydrocyclopenta[c]pyrrole-2(1H)-carboxylate and benzyl (3aR,4R,5R,6aS)-5-(2-fluorophenoxy)-3a,4-dihydroxyhexahydrocyclopenta[c]pyrrole-2(1H)-carboxylate (step 3 of Intermediate 7, 1.0 g) was separated by chiral SFC using the method below to provide benzyl (3aS,4S,5S,6aR)-5-(2-fluorophenoxy)-3a,4-dihydroxyhexahydrocyclopenta[c]pyrrole-2(1H)-carboxylate (chiral SFC Rt 13.24 min, 0.5 g) and benzyl (3aR,4R,5R,6aS)-5-(2-fluorophenoxy)-3a,4-dihydroxyhexahydrocyclopenta[c]pyrrole-2(1H)-carboxylate (chiral SFC Rt 19.13 min, 0.5 g).

Column: Chiralpak IG (10 mm×250 mm, 5 micron), Flow: 15 mL/min

Mobile phase: CO₂ (A), EtOH:IPA, 1:1 (B), Isocratic 70:30 (A:B)

### Step 2: (3aS,4S,5S,6aR)-5-(2-fluorophenoxy)hexahydrocyclopenta[c]pyrrole-3a,4(1H)-diol

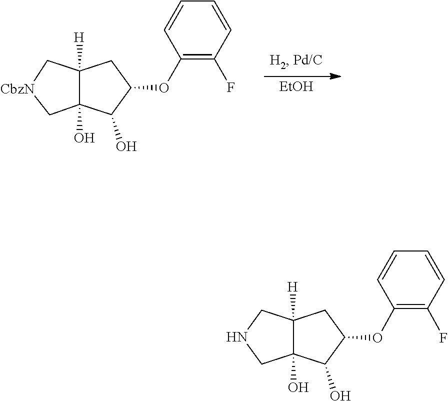 [SMILES: "EtOH" ; O=C(OCc1ccccc1)N1C[C@H]2C[C@H](Oc3ccccc3F)[C@H](O)[C@@]2(O)C1 ; O[C@H]1[C@@H](Oc2ccccc2F)C[C@@H]2CNC[C@@]21O]

Using the same method as step 5 of Intermediate 5, starting from benzyl (3aS,4S,5S,6aR)-5-(2-fluorophenoxy)-3a,4-dihydroxyhexahydrocyclopenta[c]pyrrole-2(1H)-carboxylate (chiral SFC Rt 13.24 min from step 1) (500 mg), provided the title intermediate (260 mg).

LCMS: Rt 0.11 min; MS m/z 254.3 [M+H]⁺; Method D.

¹H NMR (400 MHz, Methanol-d₄) δ 7.19 (dt, J=8.4, 1.6 Hz, 1H), 7.11-7.06 (m, 2H), 6.97-6.91 (m, 1H), 4.78-4.73 (m, 1H), 3.92 (d, J=3.2 Hz, 1H), 3.16 (dd, J=12.0, 7.6 Hz, 1H), 2.93 (d, J=12.4 Hz, 1H), 2.78 (d, J=12.0 Hz, 1H), 2.62 (dd, J=11.2, 2.8 Hz, 1H), 2.55-2.49 (m, 1H), 2.32-2.24 (m, 1H), 1.55-1.49 (m, 1H).

### Intermediate 9

### (3aR,4R,5R,6aS)-5-(2-fluorophenoxy)hexahydrocyclopenta[c]pyrrole-3a,4(1H-diol

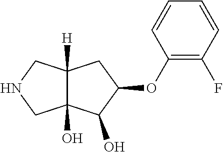 [SMILES: O[C@@H]1[C@H](Oc2ccccc2F)C[C@H]2CNC[C@]21O]

Using the same method as step 5 of Intermediate 5, starting from benzyl (3aR,4R,5R,6aS)-5-(2-fluorophenoxy)-3a,4-dihydroxyhexahydrocyclopenta[c]pyrrole-2(1H)-carboxylate (chiral SFC Rt 19.13 min from step 1 of Intermediate 8) (500 mg), provided the title intermediate (270 mg).

LCMS: Rt 0.10 min; MS m/z 254.0 [M+H]⁺; Method D.

¹H NMR (400 MHz, Methanol-d₄) δ 7.20 (dt, J=8.0, 1.6 Hz, 1H), 7.12-7.06 (m, 2H), 6.98-6.94 (m, 1H), 4.83-4.79 (m, 1H), 4.03 (d, J=4.0 Hz, 1H), 3.47-3.42 (m, 1H), 3.16-3.06 (m, 2H), 2.92-2.87 (m, 1H), 2.72-2.68 (m, 1H), 2.37-2.30 (m, 1H), 1.69-1.62 (m, 1H).

The following intermediates were made using similar procedures with the relevant starting materials:

| Intermediate | Name and structure | LCMS | ¹H NMR |
|---|---|---|---|
| 10 | 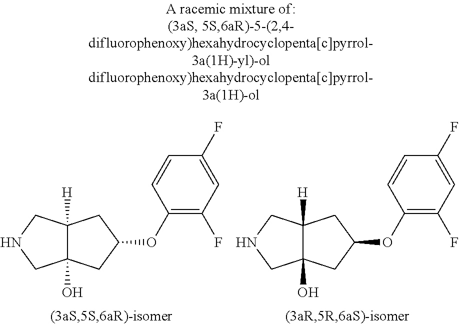 | Rt 0.73 min; MS m/z 256.3 [M + H]⁺; Method H. | (400 MHz, Methanol-d₄) δ 7.11 (td, J = 9.2, 5.4 Hz, 1H), 6.95 (ddd, J = 11.5, 8.6, 3.0 Hz, 1H), 6.91-6.77 (m, 1H), 4.81- 4.74 (m, 1H), 3.28-3.18 (m, 1H), 2.94- 2.80 (m, 2H), 2.68-2.49 (m, 2H), 2.32- 2.17 (m, 2H), 2.17-2.07 (m, 1H), 1.78- 1.66 (m, 1H). |
| 11 | 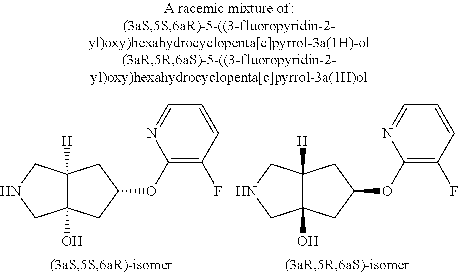 | Rt 0.70 min; MS m/z 239.0 [M + H]⁺; Method F. | |
| 12 | 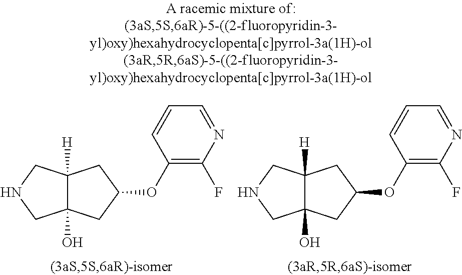 | Rt 0.09 min; MS m/z 239.4 [M + H]⁺; Method D. | |

### Intermediate 13

### 5-((4-methoxybenzyl)oxy)-2-vinylpyridine

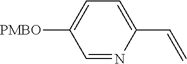

### Step 1: 2-bromo-5-((4-methoxybenzyl)oxy)pyridine

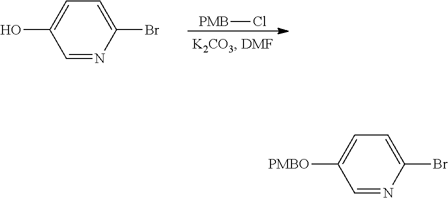

To a stirred solution of 2-bromo-5-hydroxypyridine (CAS #55717-45-8) (5.0 g, 26 mmol) in DMF (52 ml-) was added potassium carbonate (14.4 g, 104 mmol) at RT. After 10 min, the reaction was cooled to 0° C. and 4-methoxybenzyl chloride (8.13 g, 52 mmol) was added dropwise. The reaction was stirred for 16 h at RT, then quenched with water and extracted with ethyl acetate. The combined organic layer was washed with water (twice), brine, dried over Na₂SO₄, filtered and concentrated. The crude material was purified by FCC (10-20% EtOAc:Hexane) to provide the title intermediate (7.0 g) as a white solid.

LCMS: Rt 1.63 min; MS m/z 293.7 and 295.7 [M+H]⁺; Method D.

¹H NMR (400 MHz, CDCl₃) δ 8.12 (d, J=3.2 Hz, 1H), 7.37-7.32 (m, 3H), 7.14 (dd, J=8.4, 3.2 Hz, 1H), 6.93-6.91 (m, 2H), 5.01 (s, 2H), 3.82 (s, 3H).

### Step 2: 5-((4-methoxybenzyl)oxy)-2-vinylpyridine

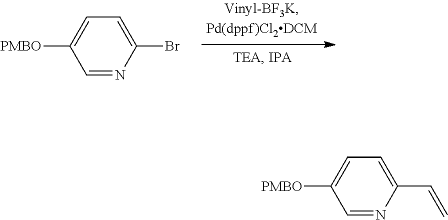

A stirred solution of 2-bromo-5-((4-methoxybenzyl)oxy)pyridine (3.0 g, 10.2 mmol), potassium vinyltrifluoroborate (2.7 g, 20 mmol) and triethylamine (4.2 mL, 30.6 mmol) in isopropanol (35 mL) was degassed with argon for 20 min. Pd(dppf)Cl₂·CH₂Cl₂ (0.83 g, 1.02 mmol) was added and the solution was again degassed with argon for 10 min, then stirred at 80° C. for 16 h. The reaction was cooled to RT, diluted with ethyl acetate and filtered through Celite, rinsing through with ethyl acetate. The filtrate was concentrated and purified by FCC (10-20% EtOAc:Hexane) to provide the title intermediate (2.1 g) as a colorless oil.

LCMS: Rt 0.72 min; MS m/z 242.4 [M+H]⁺; Method D.

¹H NMR (400 MHz, CDCl₃) δ 8.30 (d, J=2.9 Hz, 1H), 7.32 (d, J=8.7 Hz, 2H), 7.23 (d, J=3.5 Hz, 1H), 7.20-7.14 (m, 1H), 6.89 (d, J=8.7 Hz, 2H), 6.73 (dd, J=10.9, 17.5 Hz, 1H), 5.99 (dd, J=0.9, 17.6 Hz, 1H), 5.36-5.25 (m, 1H), 5.01 (s, 2H), 3.79 (s, 3H).

### Intermediate 14

### (±)-5-((4-methoxybenzyl)oxy)-2-(oxiran-2-yl)pyridine

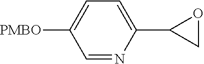

To a solution of NBS (2.4 g, 51 mmol) in dioxane (80 mL) and water (160 mL) was added AcOH (0.9 g, 2.5 mmol) followed by 5-((4-methoxybenzyl)oxy)-2-vinylpyridine (Intermediate 13, 3.3 g, 25 mmol) under argon at 0° C. The reaction was stirred for 2 h at RT to form the bromohydrin intermediate. Saturated Na₂CO₃ solution was added until the pH was fully basic, then the mixture was stirred overnight. The reaction was extracted with ethyl acetate, dried over Na₂SO₄ and concentrated. The crude material was purified by FCC (3% EtOAc:Hexane) to provide the title intermediate (1.3 g) as a brown solid.

¹H NMR (300 MHz, CDCl₃) δ 8.31 (d, J=3.0 Hz, 1H), 7.36-7.31 (m, 2H), 7.23 (dd, J=4.8, 1.8 Hz, 1H), 7.15 (d, J=9.0 Hz, 1H), 6.94-6.89 (m, 2H), 5.02 (s, 2H), 3.97-3.95 (m, 1H), 3.81 (s, 3H), 3.14 (dd, J=6.0, 4.8 Hz, 1H), 2.94 (dd, J=5.7, 2.4 Hz, 1H).

### Intermediate 15

### (±)-3-(benzyloxy)-2-fluoro-6-(oxiran-2-yl)pyridine

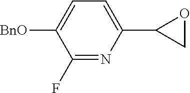 [SMILES: Fc1nc(C2CO2)ccc1OCc1ccccc1]

### Step 1: 6-bromo-2-fluoropyridin-3-ol

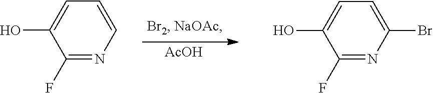 [SMILES: "AcOH" ; Oc1cccnc1F ; Oc1ccc(Br)nc1F]

To a solution of 2-fluoropyridin-3-ol (CAS #174669-74-0) (15.0 g, 44.2 mmol) in acetic acid (130 mL) was added sodium acetate (12.0 g, 48.6 mmol), followed by dropwise addition of bromine (21.3 g, 44.2 mmol) at 0° C. The reaction was stirred at RT for 1 h, then poured into ice-cold water (100 mL) and neutralized with 1M NaOH solution. The crude solution was extracted with ethyl acetate 3×, washed with brine, dried over Na₂SO₄, filtered and concentrated to provide the title intermediate as a yellow oil (24.0 g) which was used in the next step without purification.

### Step 2: 3-(benzyloxy)-6-bromo-2-fluoropyridine

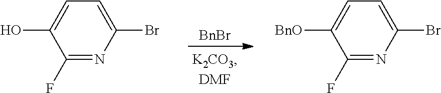 [SMILES: Oc1ccc(Br)nc1F ; "BnBr" ; Fc1nc(Br)ccc1OCc1ccccc1]

To a solution of 6-bromo-2-fluoropyridin-3-ol (24 g, 125 mmol) in DMF (150 mL) was added K₂CO₃ (69 g, 500 mmol), followed by benzyl bromide (30 mL, 250 mmol) at 0° C. The reaction was stirred at RT for 12 h, then diluted with water and extracted with EtOAc (3×50 mL). The combined organic layers were washed with brine, dried over Na₂SO₄, filtered and concentrated. The crude material was purified by FCC (0-1% EtOAc:Hexane) to provide the title intermediate (17.0 g) as a white solid.

LCMS: Rt 2.16 min; MS m/z 282.1 and 284.0 [M+H]⁺; Method E.

### Step 3: 3-(benzyloxy)-2-fluoro-6-vinylpyridine

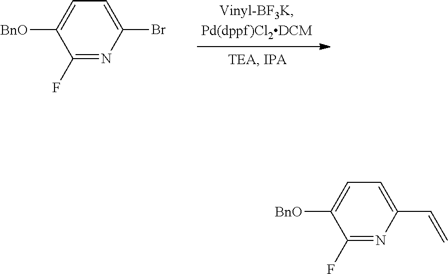 [SMILES: Fc1nc(Br)ccc1OCc1ccccc1 ; C=Cc1ccc(OCc2ccccc2)c(F)n1]

Using the same method as step 2 of Intermediate 13, starting with 3-(benzyloxy)-6-bromo-2-fluoropyridine (17 g, 60.5 mmol) and purifying with FCC (5-10% EtOAc:Hexane), provided the title intermediate (9.0 g) as a white solid.

LCMS: Rt 1.60 min; MS m/z 229.9 [M+H]⁺; Method F.

### Step 4: (±)-3-(benzyloxy)-2-fluoro-6-(oxiran-2-yl)pyridine

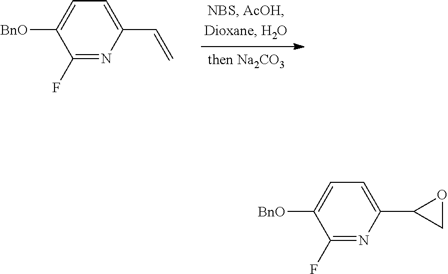 [SMILES: C=Cc1ccc(OCc2ccccc2)c(F)n1 ; Fc1nc(C2CO2)ccc1OCc1ccccc1]

Using the same method as Intermediate 14, starting with 3-(benzyloxy)-2-fluoro-6-vinylpyridine (9.0 g, 39 mmol), but with the first half of the reaction run for 48 h and the second half for 72 h, and purifying by FCC (12% EtOAc:Hexane), provided the title intermediate (4.1 g) as an off white solid.

LCMS: Rt 1.56 min; MS m/z 245.9 [M+H]⁺; Method F.

### Intermediates 16 and 17

### (S)-3-(benzyloxy)-2-fluoro-6-(oxiran-2-yl)pyridine

### (R)-3-(benzyloxy)-2-fluoro-6-(oxiran-2-yl)pyridine

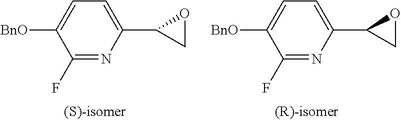 [SMILES: Fc1nc([C@H]2CO2)ccc1OCc1ccccc1 ; Fc1nc([C@@H]2CO2)ccc1OCc1ccccc1]

The two enantiomers of (±)-3-(benzyloxy)-2-fluoro-6-(oxiran-2-yl)pyridine (Intermediate 15, 1.5 g, 6.1 mmol) were separated using the following chiral HPLC method:

Column: C-4, Flow: 20 mL/min

Mobile phase: Hexane (A), IPA:EtOH 1:1 (B), Isocratic 80:20 (A:B)

Intermediate 16: chiral HPLC Rt 4.78 min, 0.6 g.

Intermediate 17: chiral HPLC Rt 5.48 min, 0.6 g.

### Intermediate 18

### (±)-3-(benzyloxy)-2-chloro-6-(oxiran-2-yl)pyridine

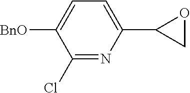 [SMILES: Clc1nc(C2CO2)ccc1OCc1ccccc1]

### Step 1: 2-chloro-6-(hydroxymethyl)pyridin-3-ol

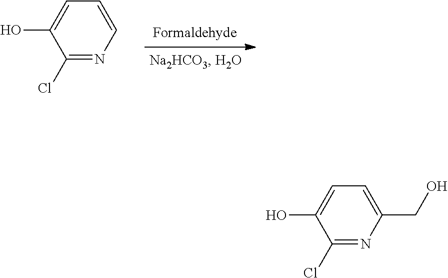 [SMILES: Oc1cccnc1Cl ; OCc1ccc(O)c(Cl)n1]

2-chloropyridin-3-ol (CAS #6636-78-8) (15 g, 115 mmol) and NaHCO₃ (14.5 g, 172 mmol) were dissolved in water (120 mL) and heated to 90° C. Aqueous formaldehyde (37%, 30 mL) was added dropwise. The reaction was heated at 90° C. for 16 h, then cooled to 0° C. and acidified with 6N HCl until pH=1. The solution was stirred at 0° C. for 1 h and then filtered. The filtrate was extracted with ethyl acetate 3×, dried over Na₂SO₄, filtered and concentrated to provide the title intermediate (15 g) which was used in the next step without purification.

LCMS: Rt 0.41 min; MS m/z 160.0 [M+H]⁺; Method F.

### Step 2: (5-(benzyloxy)-6-chloropyridin-2-yl)methanol

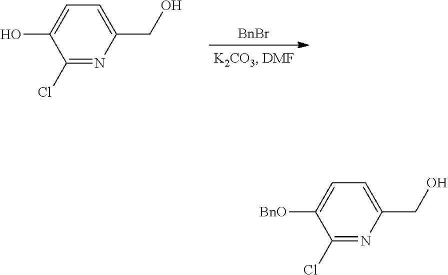

Using the same method as step 2 of Intermediate 15, starting with 2-chloro-6-(hydroxymethyl)pyridin-3-ol (10 g, 63 mmol) and purifying with FCC (20% EtOAc:Hexane), provided the title intermediate (12 g).

LCMS: Rt 2.20 min; MS m/z 250.1 [M+H]⁺; Method D.

¹H NMR (400 MHz, CDCl₃) δ 7.45-7.34 (m, 5H), 7.26-7.22 (m, 1H), 7.17 (d, J=8.4 Hz, 1H), 5.18 (s, 2H), 4.67 (d, J=5.6 Hz, 2H).

### Step 3: 5-(benzyloxy)-6-chloropicolinaldehyde

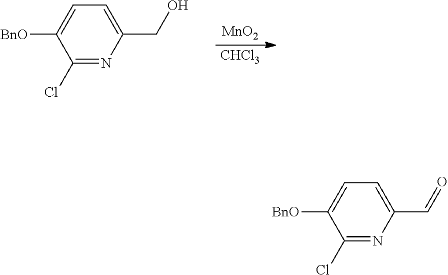

To a solution of (5-(benzyloxy)-6-chloropyridin-2-yl)methanol (9.0 g, 36 mmol) in CHCl₃ (300 mL) was added MnO₂ (15.7 g, 181 mmol) at RT, and the reaction was refluxed for 10 h. The reaction mixture was filtered through Celite, washing through with chloroform. The filtrate was concentrated to provide the title intermediate (6.0 g) which was used in the next step without purification.

¹H NMR (300 MHz, CDCl₃) δ 9.91 (s, 1H), 7.88 (d, J=6.9 Hz, 1H), 7.48-7.26 (m, 6H), 5.28 (s, 2H).

### Step 4: (±)-3-(benzyloxy)-2-chloro-6-(oxiran-2-yl)pyridine

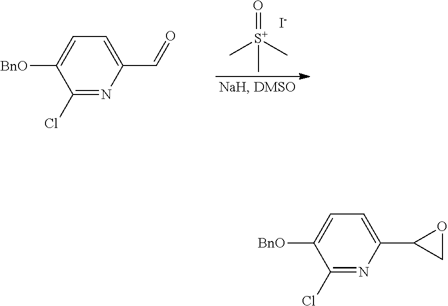

To a stirred suspension of NaH (60% in mineral oil, 1.42 g, 35.5 mmol) in DMSO (20.0 mL) was added trimethylsulfoxonium iodide (7.1 g, 32 mmol) at 0° C. and the reaction was stirred for 15 minutes at 0° C. A solution of 5-(benzyloxy)-6-chloropicolinaldehyde (4.0 g, 16 mmol) in DMSO (20.0 mL) was added dropwise and the solution was stirred for 45 min at RT. The reaction was quenched slowly with ice cold water, extracted with ethyl acetate 3×, washed with brine, dried over Na₂SO₄, filtered and concentrated. The crude material was purified by FCC (20% EtOAc:Hexane) to provide the title intermediate (1.2 g).

LCMS: Rt 1.62 min; MS m/z 262.1 [M+H]⁺; Method D.

¹H NMR (400 MHz, CDCl₃) δ 7.47-7.36 (m, 5H), 7.23 (d, J=8.4 Hz, 1H), 7.11 (d, J=8.0 Hz, 1H), 5.20 (s, 2H), 3.97-3.95 (m, 1H), 3.15 (dd, J=5.6, 4.0 Hz, 1H), 2.91 (dd, J=5.6, 2.4 Hz, 1H).

### Intermediate 19

### (±)-2-chloro-3-((4-methoxybenzyl)oxy)-6-(oxiran-2-yl)pyridine

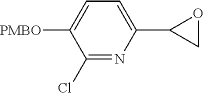

This was synthesized in a similar manner as Intermediate 18, using 4-methoxybenzylchloride instead of benzyl bromide in step 2.

LCMS: Rt 1.56 min; MS m/z 292.1 [M+H]⁺; Method D.

### Intermediate 20

### (±)-2-(4-(benzyloxy)-3,5-difluorophenyl)oxirane

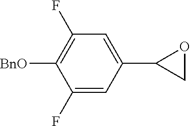 [SMILES: Fc1cc(C2CO2)cc(F)c1OCc1ccccc1]

This was made following the methods of Intermediates 13 and 14, using benzyl bromide in step 1.

¹H NMR (400 MHz, CDCl₃) δ 7.45-7.42 (m, 2H), 7.39-7.31 (m, 3H), 6.85-6.78 (m, 2H), 5.16 (s, 2H), 3.77-3.75 (m, 1H), 3.13-3.10 (m, 1H), 2.71-2.69 (m, 1H).

### Intermediate 21

### 2-bromo-1-(4-hydroxyphenyl)ethan-1-one

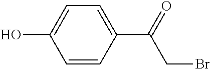 [SMILES: O=C(CBr)c1ccc(O)cc1]

To a solution of 1-(4-hydroxyphenyl)ethan-1-one (CAS #99-93-4) (3.0 g, 22 mmol) in AcOH (30 mL) was added pyridinium tribromide (7.0 g, 22 mmol) and the reaction was stirred at RT for 3 h. The mixture was adjusted to pH 6-7 and extracted with EtOAc (2×100 mL), dried with Na₂SO₄, filtered and concentrated. The crude material was purified by FCC (5-10% EtOAc:PE) to provide the title intermediate (1.0 g) as a yellow solid.

### Intermediate 22

### 2-bromo-1-(3,5-difluoro-4-hydroxyphenyl)ethan-1-one

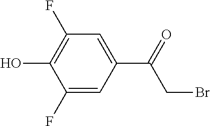 [SMILES: O=C(CBr)c1cc(F)c(O)c(F)c1]

### Step 1: 2,6-difluorophenyl acetate

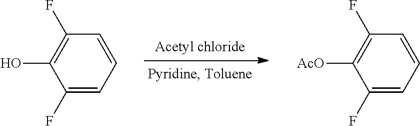 [SMILES: Oc1c(F)cccc1F ; CC(=O)Oc1c(F)cccc1F]

To a solution of 2,6-difluorophenol (CAS #28177-48-2) (15 g, 115 mmol) in toluene (130 mL) and pyridine (30 mL, 368 mmol) was added acetyl chloride (25 g, 24 mL, 322 mmol) dropwise at RT and the reaction was stirred for 1 h. The reaction was diluted with water (300 mL) and the organic layer was separated and concentrated. The residue was diluted with EtOAc (200 mL) and washed with 1M HCl (2×100 mL), then with 5% aq. K₂CO₃ (100 mL). The organic layer was dried with Na₂SO₄, filtered and concentrated to provide the title intermediate (15 g) as a yellow oil which was used without further purification.

¹H NMR (400 MHz, CDCl₃) δ 7.23-7.12 (m, 1H), 7.03-6.90 (m, 2H), 2.38 (s, 3H).

### Step 2: 1-(3,5-difluoro-4-hydroxyphenyl)ethan-1-one

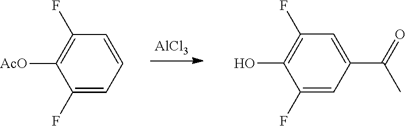

To 2,6-difluorophenyl acetate (15 g, 87.1 mmol) was added AlCl₃ (24.4 g, 183 mmol) and the mixture was stirred at 150° C. for 1 h. The reaction was cooled and poured onto ice-cold 3N HCl (300 mL). A yellow solid formed which was filtered, dissolved in EtOAc (200 mL) and washed with brine (100 mL). The organic layer was dried with Na₂SO₄, filtered and concentrated to provide the title intermediate (11 g) as a yellow solid which was used without further purification.

¹H NMR (300 MHz, CDCl₃) δ 7.64-7.48 (m, 2H), 5.97 (s, 1H), 2.56 (s, 3H).

### Step 3: 2-bromo-1-(3,5-difluoro-4-hydroxyphenyl)ethan-1-one

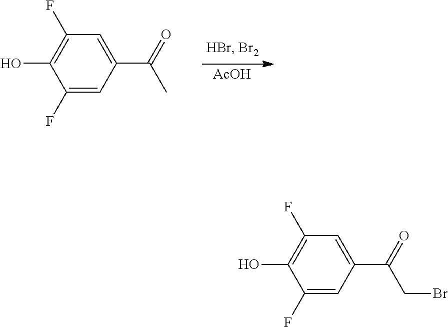

To a solution of 1-(3,5-difluoro-4-hydroxyphenyl)ethan-1-one (5 g, 29 mmol) in AcOH (80 mL) was added 35% HBr in AcOH (6.7 g, 29 mmol). Br₂ (4.6 g, 29 mmol) was added dropwise and the reaction was stirred at RT for 30 min. The reaction was quenched with saturated aq. NaHCO₃ (300 mL), extracted with EtOAc (3×50 mL), dried with Na₂SO₄, filtered and concentrated. The residue was diluted with 5:1 PE:EtOAc, stirred at RT for 30 min, then filtered. The filtrate was concentrated to provide the title intermediate (4 g) as a yellow solid which was used without further purification.

¹H NMR (400 MHz, CDCl₃) δ 7.66-7.55 (m, 2H), 4.35 (s, 2H).

### Intermediate 23

### 2-(benzyloxy)-5-(2-bromoethyl)-1,3-difluorobenzene

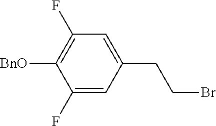 [SMILES: Fc1cc(CCBr)cc(F)c1OCc1ccccc1]

### Step 1: 2-(4-(benzyloxy)-3,5-difluorophenyl)ethan-1-ol

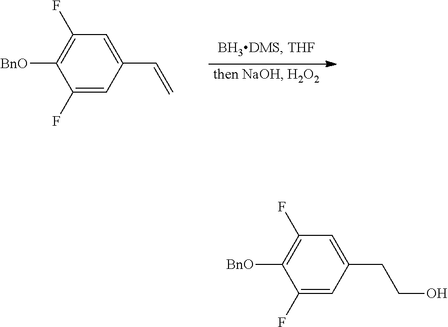

To a solution of 2-(benzyloxy)-1,3-difluoro-5-vinylbenzene (from Step 2 of Intermediate 20) (150 mg, 0.607 mmol) in THF (10 mL) was added borane dimethylsulfide (93 mg, 1.2 mmol) dropwise at 0° C. The reaction was stirred at RT for 16 h, then 3N aqueous NaOH solution (1.2 mL, 3.6 mmol) and H₂O₂ (30%, 0.375 mL, 3.6 mmol) were added and the mixture was stirred at 55° C. for 4 h. The reaction was cooled, extracted with EtOAc, dried with Na₂SO₄, filtered and concentrated. The crude material was purified by FCC (10% EtOAc:Hexane) to provide the title intermediate (70 mg).

¹H NMR (400 MHz, CDCl₃) δ 7.47-7.44 (m, 2H), 7.39-7.31 (m, 3H), 6.77 (d, J=8.8 Hz, 2H), 5.13 (s, 2H), 3.82 (t, J=6.4 Hz, 2H), 2.77 (t, J=6.0 Hz, 2H).

### Step 2: 2-(benzyloxy)-5-(2-bromoethyl)-1,3-difluorobenzene

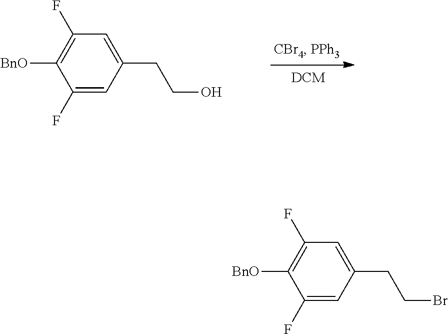 [SMILES: OCCc1cc(F)c(OCc2ccccc2)c(F)c1 ; Fc1cc(CCBr)cc(F)c1OCc1ccccc1]

To a solution of 2-(4-(benzyloxy)-3,5-difluorophenyl)ethan-1-ol (70 mg, 0.26 mmol) in DCM (10 mL) at 0° C. were added triphenylphosphine (104 mg, 0.396 mmol) and tetrabromomethane (131 mg, 0.396 mmol). The reaction was stirred for 2 h at RT, then concentrated. The crude material was purified by FCC (2% EtOAc:Hexane) to provide the title intermediate (20.0 mg).

¹H NMR (400 MHz, CDCl₃) δ 7.46-7.44 (m, 2H), 7.39-7.31 (m, 3H), 6.78-6.72 (m, 2H), 5.15 (s, 2H), 3.52 (t, J=6.8 Hz, 2H), 3.07 (t, J=7.6 Hz, 2H).

### Intermediate 24

### 1-(benzyloxy)-4-(2-bromoethyl)benzene

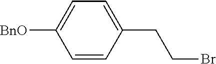 [SMILES: BrCCc1ccc(OCc2ccccc2)cc1]

### Step 1: Methyl 2-(4-(benzyloxy)phenyl)acetate

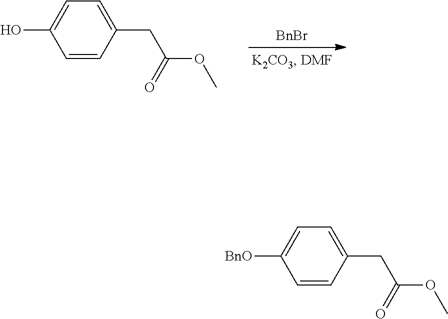 [SMILES: COC(=O)Cc1ccc(O)cc1 ; "BnBr" ; COC(=O)Cc1ccc(OCc2ccccc2)cc1]

Using the same method as step 2 of Intermediate 15, starting with methyl 2-(4-hydroxyphenyl) acetate (CAS #14199-15-6) (5.0 g, 30 mmol), provided the title intermediate (6.5 g).

¹H NMR (400 MHz, CDCl₃) δ 7.45-7.31 (m, 5H), 7.21-7.19 (m, 2H), 6.96-6.92 (m, 2H), 5.05 (s, 2H), 3.69 (s, 3H), 3.57 (s, 2H).

### Step 2: 2-(4-(benzyloxy)phenyl)ethan-1-ol

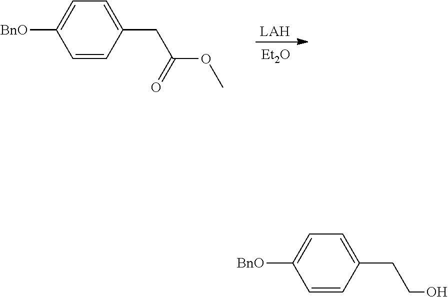 [SMILES: "Et2O" ; COC(=O)Cc1ccc(OCc2ccccc2)cc1 ; "LAH" ; OCCc1ccc(OCc2ccccc2)cc1]

To a solution of methyl 2-(4-(benzyloxy)phenyl)acetate (2.5 g, 9.8 mmol) in diethyl ether (80 mL) was added LiAlH₄ (1 M in THF, 19.5 mL, 19.5 mmol) at 0° C. and the reaction was stirred at RT for 16 h. The reaction was quenched with water and extracted with ethyl acetate. The organic layer was dried with Na₂SO₄, filtered and concentrated to provide the title intermediate (2.0 g) which was used without further purification.

¹H NMR (400 MHz, CDCl₃) δ 7.46-7.30 (m, 5H), 7.18-7.12 (m, 2H), 6.97-6.90 (m, 2H), 5.05 (s, 2H), 3.83 (q, J=6.4 Hz, 2H), 2.82 (t, J=6.4 Hz, 2H), 1.38 (t, J=6.4 Hz, 1H).

### Step 3: 1-(benzyloxy)-4-(2-bromoethyl)benzene

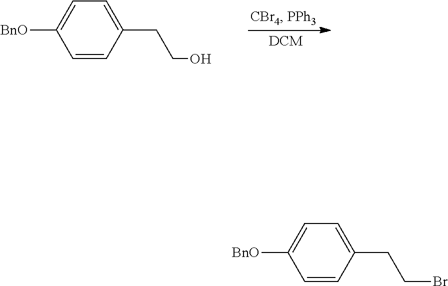

Using the same method as step 2 of Intermediate 23, starting with 2-(4-(benzyloxy)phenyl)ethan-1-ol (2.0 g, 8.8 mmol), provided the title intermediate (1.5 g).

¹H NMR (400 MHz, CDCl₃) δ 7.45-7.32 (m, 5H), 7.15-7.12 (m, 2H), 6.96-6.92 (m, 2H), 5.06 (s, 2H), 3.54 (t, J=8.0 Hz, 2H), 3.11 (t, J=8.0 Hz, 2H).

### Examples 1A, 1B, 1C and 1D

(3aS,5S,6aR)-5-(2-fluorophenoxy)-2-((S)-2-hydroxy-2-(5-hydroxypyridin-2-yl)ethyl)hexahydrocyclopenta[c]pyrrol-3a(1H)-ol
(3aS,5S,6aR)-5-(2-fluorophenoxy)-2-((R)-2-hydroxy-2-(5-hydroxypyridin-2-yl)ethyl)hexahydrocyclopenta[c]pyrrol-3a(1H)-ol
(3aR,5R,6aS)-5-(2-fluorophenoxy)-2-((S)-2-hydroxy-2-(5-hydroxypyridin-2-yl)ethyl)hexahydrocyclopenta[c]pyrrol-3a(1H)-ol
(3aR,5R,6aS)-5-(2-fluorophenoxy)-2-((R)-2-hydroxy-2-(5-hydroxypyridin-2-yl)ethyl)hexahydrocyclopenta[c]pyrrol-3a(1H)-ol

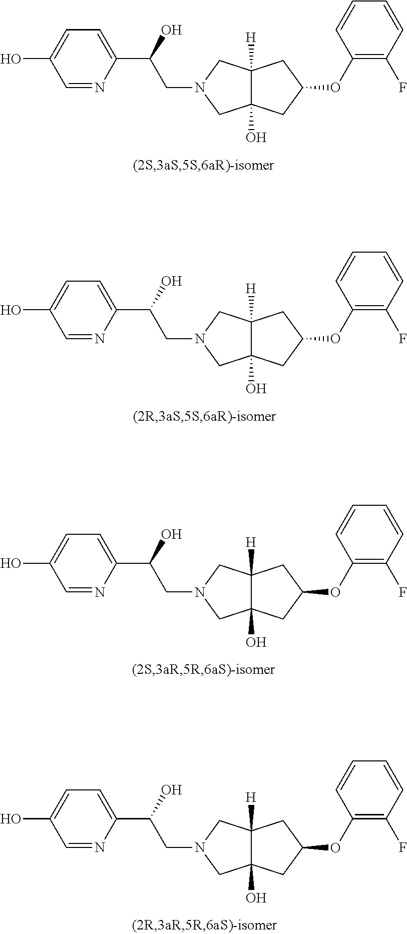 [SMILES: Oc1ccc([C@@H](O)CN2C[C@H]3C[C@H](Oc4ccccc4F)C[C@@]3(O)C2)nc1 ; Oc1ccc([C@H](O)CN2C[C@H]3C[C@H](Oc4ccccc4F)C[C@@]3(O)C2)nc1 ; Oc1ccc([C@@H](O)CN2C[C@@H]3C[C@@H](Oc4ccccc4F)C[C@]3(O)C2)nc1 ; Oc1ccc([C@H](O)CN2C[C@@H]3C[C@@H](Oc4ccccc4F)C[C@]3(O)C2)nc1]

### Step 1: A Mixture of: (3aS,5S,6aR)-5-(2-fluorophenoxy)-2-((S)-2-hydroxy-2-(5-((4-methoxybenzyl)oxy)pyridin-2-yl)ethyl)hexahydrocyclopenta[c]pyrrol-3a(1H)-ol (3aS,5S,6aR)-5-(2-fluorophenoxy)-2-((R)-2-hydroxy-2-(5-((4-methoxybenzyl)oxy)pyridin-2-yl)ethyl)hexahydrocyclopenta[c]pyrrol-3a(1H)-ol (3aR,5R,6aS)-5-(2-fluorophenoxy)-2-((S)-2-hydroxy-2-(5-((4-methoxybenzyl)oxy)pyridin-2-yl)ethyl)hexahydrocyclopenta[c]pyrrol-3a(1H)-ol (3aR,5R,6aS)-5-(2-fluorophenoxy)-2-((R)-2-hydroxy-2-(5-((4-methoxybenzyl)oxy)pyridin-2-yl)ethyl)hexahydrocyclopenta[c]pyrrol-3a(1H)-ol

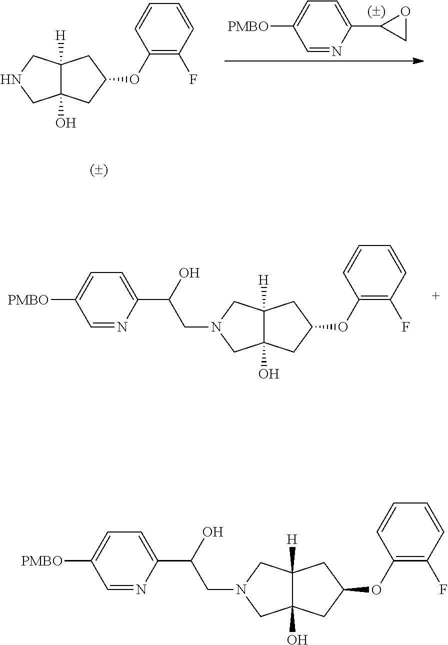

A solution of Intermediate 3 (120 mg, 0.505 mmol) and Intermediate 14 (130 mg, 0.505 mmol) in EtOH (10 mL) was stirred at 90° C. for 4 h. The reaction was cooled, concentrated, and purified by FCC (5% MeOH:DCM) to provide the title intermediate (130 mg).

LCMS: Rt 0.48 min; MS m/z 495.2 [M+H]⁺; Method D.

### Step 2: A Mixture of: (3aS,5S,6aR)-5-(2-fluorophenoxy)-2-((S)-2-hydroxy-2-(5-hydroxypyridin-2-yl)ethyl)hexahydrocyclopenta[c]pyrrol-3a(1H)-ol (3aS,5S,6aR)-5-(2-fluorophenoxy)-2-((R)-2-hydroxy-2-(5-hydroxypyridin-2-yl)ethyl)hexahydrocyclopenta[c]pyrrol-3a(1H)-ol (3aR,5R,6aS)-5-(2-fluorophenoxy)-2-((S)-2-hydroxy-2-(5-hydroxypyridin-2-yl)ethyl)hexahydrocyclopenta[c]pyrrol-3a(1H)-ol (3aR,5R,6aS)-5-(2-fluorophenoxy)-2-((R)-2-hydroxy-2-(5-hydroxypyridin-2-yl)ethyl)hexahydrocyclopenta[c]pyrrol-3a(1H-ol

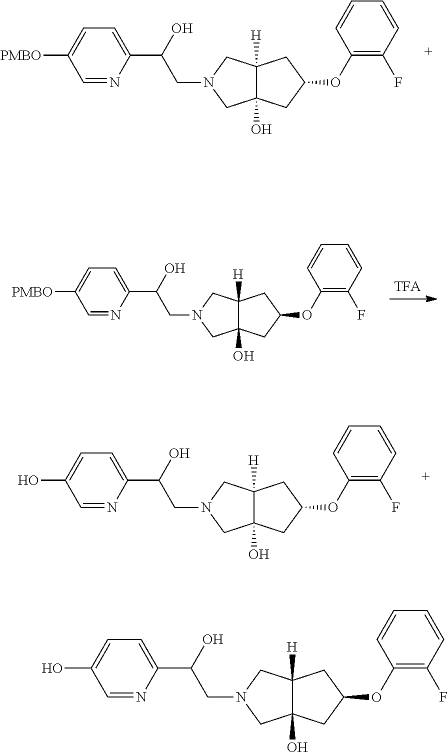

A mixture of (3aS,5S,6aR)-5-(2-fluorophenoxy)-2-((S)-2-hydroxy-2-(5-((4-methoxybenzyl)oxy)pyridin-2-yl)ethyl)hexahydrocyclopenta[c]pyrrol-3a(1H)-ol, (3aS,5S,6aR)-5-(2-fluorophenoxy)-2-((R)-2-hydroxy-2-(5-((4-methoxybenzyl)oxy)pyridin-2-yl)ethyl)hexahydrocyclopenta[c]pyrrol-3a(1H)-ol, (3aR,5R,6aS)-5-(2-fluorophenoxy)-2-((S)-2-hydroxy-2-(5-((4-methoxybenzyl)oxy)pyridin-2-yl)ethyl)hexahydrocyclopenta[c]pyrrol-3a(1H)-ol, and (3aR,5R,6aS)-5-(2-fluorophenoxy)-2-((R)-2-hydroxy-2-(5-((4-methoxybenzyl)oxy)pyridin-2-yl)ethyl)hexahydrocyclopenta[c]pyrrol-3a(1H)-ol (130 mg, 0.262 mmol) in TFA (3 mL) was stirred at RT for 4 h. The solution was concentrated and the crude material was purified using the HPLC condition below to provide the title intermediate (40 mg).

Column: Kinetex (150 mm×21.2 mm), 5.0 μm, Flow: 20 ml/min

Mobile phase: 0.05% TFA in water (A), Acetonitrile (B)

LCMS: Rt 0.12 min; MS m/z 375.2 [M+H]⁺; Method D.

### Step 3: Chiral Separation

The four diastereomers were separated using the following chiral HPLC condition:

Column: Chiralpak IC (10 mm×250 mm, 5 micron), Flow: 9 mL/min

Mobile phase: Hexane (A), 0.1% DEA in EtOH:MeOH, 80:20 (B), Isocratic 94:6 (A:B)

Example 1A (chiral HPLC Rt 5.68 min): 6.0 mg.

LCMS: Rt 0.12 min; MS m/z 375.1 [M+H]⁺; Method D.

¹H NMR (400 MHz, Methanol-d₄) δ 8.02 (d, J=2.4 Hz, 1H), 7.41 (d, J=8.4 Hz, 1H), 7.23 (dd, J=8.8, 2.8 Hz, 1H), 7.09-7.00 (m, 3H), 6.94-6.88 (m, 1H), 4.85-4.77 (m, 2H), 3.00 (t, J=8.0 Hz, 1H), 2.90-2.77 (m, 4H), 2.61-2.51 (m, 2H), 2.29 (dd, J=13.2, 4.8 Hz, 1H), 2.23-2.15 (m, 1H), 2.05 (dd, J=14.0, 5.6 Hz, 1H), 1.85-1.79 (m, 1H).

Example 1B (chiral HPLC Rt 8.59 min): 5.6 mg.

LCMS: Rt 0.70 min; MS m/z 375.2 [M+H]⁺; Method G.

¹H NMR (400 MHz, Methanol-d₄) δ 8.01 (d, J=2.8 Hz, 1H), 7.40 (d, J=8.4 Hz, 1H), 7.22 (dd, J=8.4, 2.8 Hz, 1H), 7.08-6.99 (m, 3H), 6.93-6.90 (m, 1H), 4.82-4.76 (m, 2H), 2.97 (t, J=8.0 Hz, 1H), 2.86 (d, J=9.6 Hz, 1H), 2.78-2.77 (m, 2H), 2.64 (d, J=9.6 Hz, 1H), 2.55-2.49 (m, 2H), 2.26-2.17 (m, 2H), 2.05 (dd, J=13.6, 6.0 Hz, 1H), 1.85-1.81 (m, 1H).

Example 1C (chiral HPLC Rt 4.93 min): 5.5 mg.

LCMS: Rt 0.83 min; MS m/z 375.0 [M+H]⁺; Method G.

¹H NMR (400 MHz, Methanol-d₄) δ 8.02 (d, J=2.4 Hz, 1H), 7.41 (d, J=8.4 Hz, 1H), 7.23 (dd, J=8.8, 2.8 Hz, 1H), 7.09-7.00 (m, 3H), 6.94-6.88 (m, 1H), 4.85-4.77 (m, 2H), 3.00 (t, J=8.0 Hz, 1H), 2.90-2.77 (m, 4H), 2.61-2.51 (m, 2H), 2.29 (dd, J=13.2, 4.8 Hz, 1H), 2.23-2.15 (m, 1H), 2.05 (dd, J=14.0, 6.0 Hz, 1H), 1.83-1.77 (m, 1H).

Example 1D (chiral HPLC Rt 3.90 min): 6.0 mg.

LCMS: Rt 0.70 min; MS m/z 375.0 [M+H]⁺; Method G.

¹H NMR (400 MHz, Methanol-d₄) δ 8.00 (d, J=2.4 Hz, 1H), 7.40 (d, J=8.4 Hz, 1H), 7.22 (dd, J=8.4, 3.2 Hz, 1H), 7.08-6.98 (m, 3H), 6.93-6.88 (m, 1H), 4.80-4.74 (m, 2H), 2.90-2.81 (m, 2H), 2.73-2.63 (m, 2H), 2.55 (d, J=9.2 Hz, 1H), 2.50-2.46 (m, 2H), 2.24-2.16 (m, 2H), 2.04 (dd, J=13.2, 6.0 Hz, 1H), 1.86-1.80 (m, 1H).

### Examples 2A, 2B, 2C and 2D

(3aS,4S,5S,6aR)-5-(2-fluorophenoxy)-2-((S)-2-hydroxy-2-(5-hydroxypyridin-2-yl)ethyl)hexahydrocyclopenta[c]pyrrole-3a,4(1H)-diol
(3aS,4S,5S,6aR)-5-(2-fluorophenoxy)-2-((R)-2-hydroxy-2-(5-hydroxypyridin-2-yl)ethyl)hexahydrocyclopenta[c]pyrrole-3a,4(1H)-diol
(3aR,4R,5R,6aS)-5-(2-fluorophenoxy)-2-((S)-2-hydroxy-2-(5-hydroxypyridin-2-yl)ethyl)hexahydrocyclopenta[c]pyrrole-3a,4(1H)-diol
(3aR,4R,5R,6aS)-5-(2-fluorophenoxy)-2-((R)-2-hydroxy-2-(5-hydroxypyridin-2-yl)ethyl)hexahydrocyclopenta[c]pyrrole-3a,4(1H)-diol

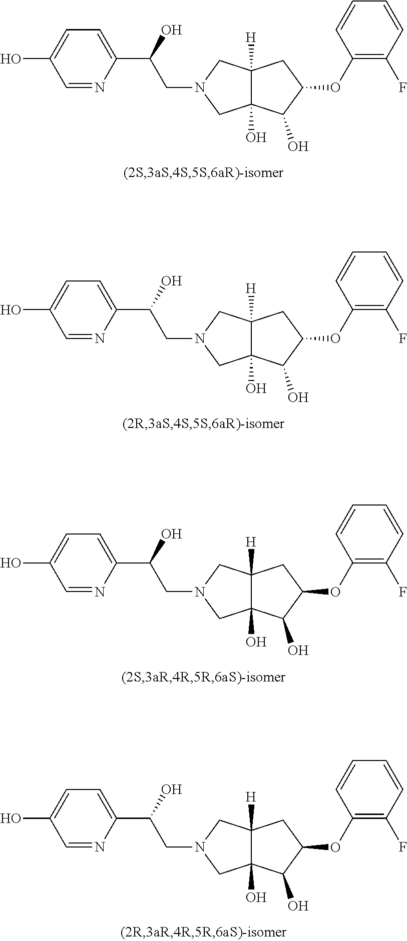 [SMILES: Oc1ccc([C@@H](O)CN2C[C@H]3C[C@H](Oc4ccccc4F)[C@H](O)[C@@]3(O)C2)nc1 ; Oc1ccc([C@H](O)CN2C[C@H]3C[C@H](Oc4ccccc4F)[C@H](O)[C@@]3(O)C2)nc1 ; Oc1ccc([C@@H](O)CN2C[C@@H]3C[C@@H](Oc4ccccc4F)[C@@H](O)[C@]3(O)C2)nc1 ; Oc1ccc([C@H](O)CN2C[C@@H]3C[C@@H](Oc4ccccc4F)[C@@H](O)[C@]3(O)C2)nc1]

### Step 1 (Intermediates 25A and 25B): A Mixture of: (3aS,4S,5S,6aR)-5-(2-fluorophenoxy)-2-((S)-2-hydroxy-2-(5-((4-methoxybenzyl)oxy)pyridin-2-yl)ethyl)hexahydrocyclopenta[c]pyrrole-3a,4(1H)-diol (3aS,4S,5S,6aR)-5-(2-fluorophenoxy)-2-((R)-2-hydroxy-2-(5-((4-methoxybenzyl)oxy)pyridin-2-yl)ethyl)hexahydrocyclopenta[c]pyrrole-3a,4(1H)-diol

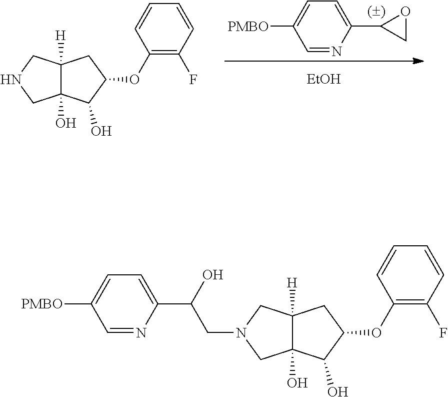

Using the same method as step 1 of Example 1A/1B/1C/1D, starting with Intermediate 8 (125 mg, 0.49 mmol) and Intermediate 14 (151 mg, 0.58 mmol), provided the title intermediates (150 mg).

LCMS: Rt 0.26 min; MS m/z 511.2 [M+H]⁺; Method D.

### Step 2 (Intermediates 25C and 25D): A Mixture of: (3aR,4R,5R,6aS)-5-(2-fluorophenoxy)-2-((S)-2-hydroxy-2-(5-((4-methoxybenzyl)oxy)pyridin-2-yl)ethyl)hexahydrocyclopenta[c]pyrrole-3a,4(1H)-diol (3aR,4R,5R,6aS)-5-(2-fluorophenoxy)-2-((R)-2-hydroxy-2-(5-((4-methoxybenzyl)oxy)pyridin-2-yl)ethyl)hexahydrocyclopenta[c]pyrrole-3a,4(1H)-diol

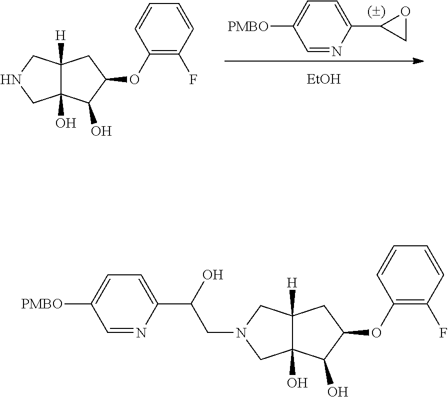

Using the same method as step 1, starting with Intermediate 9 (125 mg, 0.49 mmol) and Intermediate 14 (151 mg, 0.58 mmol), provided the title intermediates (150 mg).

LCMS: Rt 1.64 min; MS m/z 510.9 [M+H]⁺; Method E.

### Step 3 (Examples 2A and 2B)

(3aS,4S,5S,6aR)-5-(2-fluorophenoxy)-2-((S)-2-hydroxy-2-(5-hydroxypyridin-2-yl)ethyl)hexahydrocyclopenta[c]pyrrole-3a,4(1H)-diol
(3aS,4S,5S,6aR)-5-(2-fluorophenoxy)-2-((R)-2-hydroxy-2-(5-hydroxypyridin-2-yl)ethyl)hexahydrocyclopenta[c]pyrrole-3a,4(1H)-diol

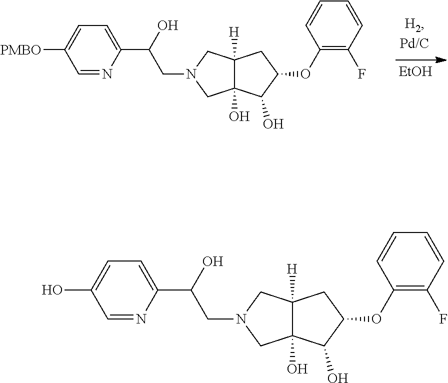

A solution of (3aS,4S,5S,6aR)-5-(2-fluorophenoxy)-2-((S)-2-hydroxy-2-(5-((4-methoxybenzyl)oxy)pyridin-2-yl)ethyl)hexahydrocyclopenta[c]pyrrole-3a,4(1H)-diol and (3aS,4S,5S,6aR)-5-(2-fluorophenoxy)-2-((R)-2-hydroxy-2-(5-((4-methoxybenzyl)oxy)pyridin-2-yl)ethyl)hexahydrocyclopenta[c]pyrrole-3a,4(1H)-diol (Intermediates 25A and 25B) (150 mg, 0.29 mmol) in EtOH (10 mL) was shaken with 10% Pd on carbon (15.0 mg) under a H₂ balloon for 6 h. The reaction was filtered through Celite, concentrated, and purified by the preparative HPLC method below to provide a mixture of Examples 2A and 2B (70 mg).

Column: Gemini-NX-C18 (250 mm×21.20 mm), 5.0 μm, Flow: 18 mL/min

Mobile phase: 0.02% TFA in water (A), Acetonitrile:MeOH 1:1 (B)

LCMS: Rt 0.12 min; MS m/z 391.1 [M+H]⁺; Method D.

The two diastereomers were separated using the following chiral SFC method: Column: Chiralpak IG 21×250 mm, 5 micron, Flow rate: 80 g/min Mobile phase: CO₂ (A), MeOH with 10 mM NH₄OH (B), Isocratic 65:35 (A:B)

Example 2A (preparative chiral SFC Rt 5.4 min): 19 mg.

Analytical chiral SFC: Rt 3.85 min (Column: Chiralpak IG 4.6×100 mm, 5 μm, flow rate 5 mL/min, mobile phase A: CO₂, phase B: Methanol with 10 mM NH₄OH, gradient: 5-55% B).

LCMS: Rt 0.40 min; MS m/z 391.2 [M+H]⁺; Method C.

¹H NMR (400 MHz, Methanol-d₄) δ 8.00 (dd, J=2.8, 0.7 Hz, 1H), 7.39 (d, J=8.6 Hz, 1H), 7.20 (dd, J=8.5, 2.8 Hz, 1H), 7.13-7.02 (m, 3H), 6.99-6.88 (m, 1H), 4.74 (t, J=6.5 Hz, 1H), 4.69 (td, J=5.5, 3.6 Hz, 1H), 3.93 (d, J=3.6 Hz, 1H), 2.96 (d, J=9.6 Hz, 1H), 2.76-2.70 (m, 2H), 2.69-2.57 (m, 2H), 2.45-2.34 (m, 2H), 2.24 (ddd, J=13.2, 9.8, 5.9 Hz, 1H), 1.54 (dt, J=13.1, 5.3 Hz, 1H).

Example 2B (preparative chiral SFC Rt 10.0 min): 16 mg.

Analytical chiral SFC: Rt 4.36 min (Column: Chiralpak IG 4.6×100 mm, 5 μm, flow rate 5 mL/min, mobile phase A: CO₂, phase B: Methanol with 10 mM NH₄OH, gradient: 5-55% B).

LCMS: Rt 0.40 min; MS m/z 391.2 [M+H]⁺; Method C.

¹H NMR (400 MHz, Methanol-d₄) δ 8.01 (dd, J=2.8, 0.7 Hz, 1H), 7.43-7.35 (m, 1H), 7.20 (dd, J=8.5, 2.8 Hz, 1H), 7.14-7.02 (m, 3H), 6.98-6.89 (m, 1H), 4.75 (t, J=6.4 Hz, 1H), 4.69 (td, J=5.4, 3.6 Hz, 1H), 3.90 (d, J=3.6 Hz, 1H), 2.97 (d, J=9.6 Hz, 1H), 2.77-2.65 (m, 3H), 2.61 (dd, J=9.2, 2.1 Hz, 1H), 2.45-2.32 (m, 2H), 2.24 (ddd, J=13.2, 9.8, 5.8 Hz, 1H), 1.55 (dt, J=13.2, 5.3 Hz, 1H).

### Step 4 (Examples 2C and 2D)

(3aR,4R,5R,6aS)-5-(2-fluorophenoxy)-2-((S)-2-hydroxy-2-(5-hydroxypyridin-2-yl)ethyl)hexahydrocyclopenta[c]pyrrole-3a,4(1H)-diol
(3aR,4R,5R,6aS)-5-(2-fluorophenoxy)-2-((R)-2-hydroxy-2-(5-hydroxypyridin-2-yl)ethyl)hexahydrocyclopenta[c]pyrrole-3a,4(1H)-diol

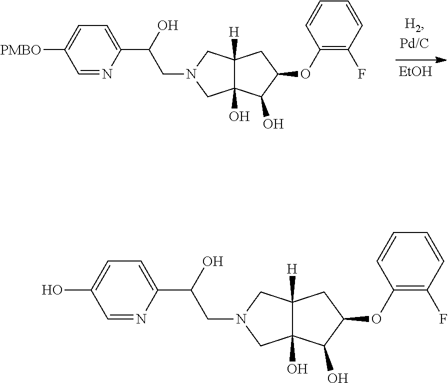

Using the same method as step 3, starting with Intermediates 25C and 25D (150 mg, 0.29 mmol), provided a mixture of Examples 2C and 2D (70 mg).

LCMS: Rt 0.47 min; MS m/z 391.1 [M+H]⁺; Method E.

The two diastereomers were separated using the following chiral SFC method:

Column: Chiralpak IG (10 mm×250 mm, 5 micron), Flow: 10 ml/min

Mobile phase: CO₂ (A), 0.1% DEA in IPA (B), Isocratic 65:35 (A:B)

Example 2C (chiral SFC Rt 5.26 min): 20 mg.

LCMS: Rt 0.39 min; MS m/z 391.1 [M+H]⁺; Method E.

¹H NMR (400 MHz, Methanol-d₄) δ 8.00 (d, J=2.4 Hz, 1H), 7.38 (d, J=8.8 Hz, 1H), 7.20 (dd, J=8.8, 3.2 Hz, 1H), 7.10-7.02 (m, 3H), 6.96-6.90 (m, 1H), 4.75 (t, J=6.4 Hz, 1H), 4.69-4.66 (m, 1H), 3.90 (d, J=4.0 Hz, 1H), 2.97 (d, J=9.6 Hz, 1H), 2.73 (d, J=6.4 Hz, 2H), 2.70-2.66 (m, 1H), 2.61 (d, J=6.0 Hz, 1H), 2.41-2.34 (m, 2H), 2.27-2.20 (m, 1H), 1.58-1.51 (m, 1H).

Example 2D (chiral SFC Rt 7.25 min): 20 mg.

LCMS: Rt 0.40 min; MS m/z 391.0 [M+H]⁺; Method E.

¹H NMR (400 MHz, Methanol-d₄) δ 7.99 (d, J=2.8 Hz, 1H), 7.38 (d, J=8.8 Hz, 1H), 7.20 (dd, J=8.8, 3.2 Hz, 1H), 7.10-7.05 (m, 3H), 6.95-6.92 (m, 1H), 4.74 (t, J=6.4 Hz, 1H), 4.69-4.66 (m, 1H), 3.92 (d, J=3.6 Hz, 1H), 2.95 (d, J=9.6 Hz, 1H), 2.73 (d, J=6.0 Hz, 2H), 2.66-2.60 (m, 2H), 2.39 (d, J=10.0 Hz, 2H), 2.27-2.20 (m, 1H), 1.56-1.51 (m, 1H).

### Examples 3A, 3B, 3C and 3D

(3aS,4S,5S,6aR)-2-((S)-2-(6-fluoro-5-hydroxypyridin-2-yl)-2-hydroxyethyl)-5-phenoxyhexahydrocyclopenta[c]pyrrole-3a,4(1H)-diol
(3aS,4S,5S,6aR)-2-((R)-2-(6-fluoro-5-hydroxypyridin-2-yl)-2-hydroxyethyl)-5-phenoxyhexahydrocyclopenta[c]pyrrole-3a,4(1H)-diol
(3aR,4R,5R,6aS)-2-((S)-2-(6-fluoro-5-hydroxypyridin-2-yl)-2-hydroxyethyl)-5-phenoxyhexahydrocyclopenta[c]pyrrole-3a,4(1H)-diol
(3aR,4R,5R,6aS)-2-((R)-2-(6-fluoro-5-hydroxypyridin-2-yl)-2-hydroxyethyl)-5-phenoxyhexahydrocyclopenta[c]pyrrole-3a,4(1H)-diol

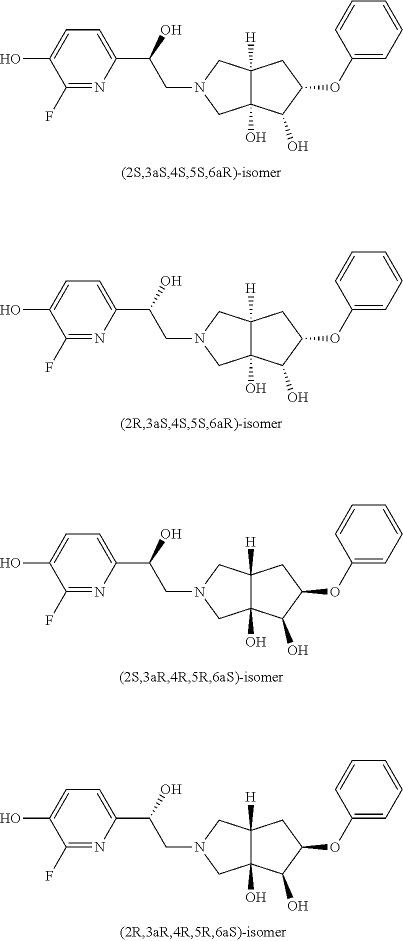 [SMILES: Oc1ccc([C@@H](O)CN2C[C@H]3C[C@H](Oc4ccccc4)[C@H](O)[C@@]3(O)C2)nc1F ; Oc1ccc([C@H](O)CN2C[C@H]3C[C@H](Oc4ccccc4)[C@H](O)[C@@]3(O)C2)nc1F ; Oc1ccc([C@@H](O)CN2C[C@@H]3C[C@@H](Oc4ccccc4)[C@@H](O)[C@]3(O)C2)nc1F ; Oc1ccc([C@H](O)CN2C[C@@H]3C[C@@H](Oc4ccccc4)[C@@H](O)[C@]3(O)C2)nc1F]

### Step 1 (Intermediates 26A, 26B, 260 and 260)

(3aS,4S,5S,6aR)-2-((S)-2-(5-(benzyloxy)-6-fluoropyridin-2-yl)-2-hydroxyethyl)-5-phenoxyhexahydrocyclopenta[c]pyrrole-3a,4(1H)-diol
(3aS,4S,5S,6aR)-2-((R)-2-(5-(benzyloxy)-6-fluoropyridin-2-yl)-2-hydroxyethyl)-5-phenoxyhexahydrocyclopenta[c]pyrrole-3a,4(1H)-diol
(3aR,4R,5R,6aS)-2-((S)-2-(5-(benzyloxy)-6-fluoropyridin-2-yl)-2-hydroxyethyl)-5-phenoxyhexahydrocyclopenta[c]pyrrole-3a,4(1H)-diol
(3aR,4R,5R,6aS)-2-((R)-2-(5-(benzyloxy)-6-fluoropyridin-2-yl)-2-hydroxyethyl)-5-phenoxyhexahydrocyclopenta[c]pyrrole-3a,4(1H)-diol

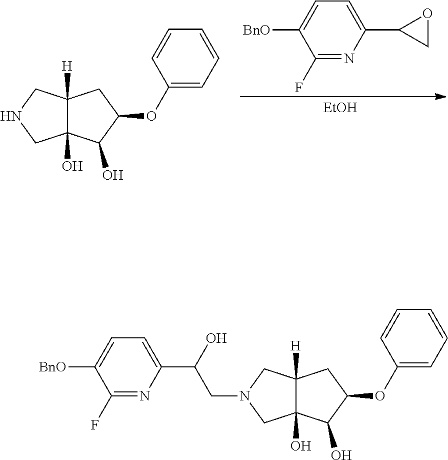 [SMILES: "EtOH" ; O[C@@H]1[C@H](Oc2ccccc2)C[C@H]2CNC[C@]21O ; Fc1nc(C2CO2)ccc1OCc1ccccc1 ; OC(CN1C[C@@H]2C[C@@H](Oc3ccccc3)[C@@H](O)[C@]2(O)C1)c1ccc(OCc2ccccc2)c(F)n1]

Using the same method as step 1 of Example 1A/1B/1C/1D, starting with Intermediate 6 (100 mg, 0.425 mmol) and Intermediate 16 (125 mg, 0.51 mmol), provided Intermediate 26A (130 mg).

LCMS: Rt 1.89 min; MS m/z 481.3 [M+H]⁺; Method E.

Using the same method, starting from Intermediate 6 (100 mg, 0.425 mmol) and Intermediate 17 (125 mg, 0.51 mmol), provided Intermediate 26B (120 mg).

LCMS: Rt 1.90 min; MS m/z 481.3 [M+H]⁺; Method E.

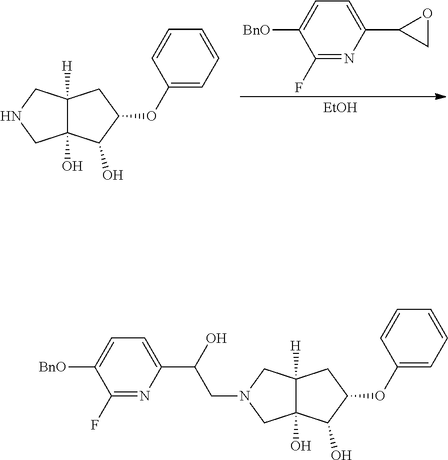 [SMILES: "EtOH" ; O[C@H]1[C@@H](Oc2ccccc2)C[C@@H]2CNC[C@@]21O ; Fc1nc(C2CO2)ccc1OCc1ccccc1 ; OC(CN1C[C@H]2C[C@H](Oc3ccccc3)[C@H](O)[C@@]2(O)C1)c1ccc(OCc2ccccc2)c(F)n1]

Using the same method, starting from Intermediate 5 (100 mg, 0.425 mmol) and Intermediate 16 (125 mg, 0.51 mmol), provided Intermediate 26C (110 mg).

LCMS: Rt 1.34 min; MS m/z 481.6 [M+H]⁺; Method F.

Using the same method, starting from Intermediate 5 (100 mg, 0.425 mmol) and Intermediate 17 (125 mg, 0.51 mmol), provided Intermediate 26D (105 mg).

LCMS: Rt 1.33 min; MS m/z 481.6 [M+H]⁺; Method F.

### Step 2 (Examples 3A, 3B, 3C, and 3D)

(3aS,4S,5S,6aR)-2-((S)-2-(6-fluoro-5-hydroxypyridin-2-yl)-2-hydroxyethyl)-5-phenoxyhexahydrocyclopenta[c]pyrrole-3a,4(1H)-diol
(3aS,4S,5S,6aR)-2-((R)-2-(6-fluoro-5-hydroxypyridin-2-yl)-2-hydroxyethyl)-5-phenoxyhexahydrocyclopenta[c]pyrrole-3a,4(1H)-diol
(3aR,4R,5R,6aS)-2-((S)-2-(6-fluoro-5-hydroxypyridin-2-yl)-2-hydroxyethyl)-5-phenoxyhexahydrocyclopenta[c]pyrrole-3a,4(1H-diol
(3aR,4R,5R,6aS)-2-((R)-2-(6-fluoro-5-hydroxypyridin-2-yl)-2-hydroxyethyl)-5-phenoxyhexahydrocyclopenta[c]pyrrole-3a,4(1H)-diol

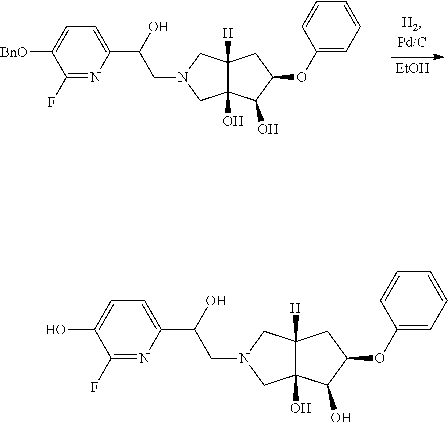 [SMILES: "EtOH" ; OC(CN1C[C@@H]2C[C@@H](Oc3ccccc3)[C@@H](O)[C@]2(O)C1)c1ccc(OCc2ccccc2)c(F)n1 ; Oc1ccc(C(O)CN2C[C@@H]3C[C@@H](Oc4ccccc4)[C@@H](O)[C@]3(O)C2)nc1F]

Example 3A: A solution of Intermediate 26A (120 mg, 0.25 mmol) in EtOH (10 mL) was shaken with 10% Pd on carbon (12 mg) under a H₂ balloon for 6 h. The reaction was filtered through Celite, concentrated, and purified by the preparative HPLC method below to provide the title compound (75 mg).

Column: Waters X Bridge C18 (150 mm×21.2 mm), 5.0 μm Mobile phase: 0.02% NH₄OH in water (A), Acetonitrile (B), Flow: 15.0 mL/min LCMS: Rt 0.11 min; MS m/z 391.2 [M+H]⁺; Method D.

¹H NMR (400 MHz, Methanol-d₄) δ 7.85-7.26 (m, 4H), 6.94-6.92 (m, 3H), 4.72-4.67 (m, 2H), 3.93 (d, J=3.6 Hz, 1H), 3.00 (d, J=10.0 Hz, 1H), 2.83-2.73 (m, 3H), 2.66-2.63 (m, 1H), 2.44 (d, J=10.0 Hz, 1H), 2.42-2.38 (m, 1H), 2.28-2.21 (m, 1H), 1.65-1.60 (m, 1H).

Example 3B: Using the same method, starting from Intermediate 26B (120 mg, 0.25 mmol), provided the title compound (75 mg).

LCMS: Rt 0.45 min; MS m/z 391.3 [M+H]⁺; Method E.

¹H NMR (400 MHz, Methanol-d₄) δ 7.34-7.24 (m, 4H), 6.94-6.91 (m, 3H), 4.73-4.67 (m, 2H), 3.95 (d, J=3.6 Hz, 1H), 3.00 (d, J=10.0 Hz, 1H), 2.86-2.74 (m, 3H), 2.66-2.63 (m, 1H), 2.50 (d, J=10.0 Hz, 1H), 2.44-2.38 (m, 1H), 2.28-2.20 (m, 1H), 1.63-1.57 (m, 1H).

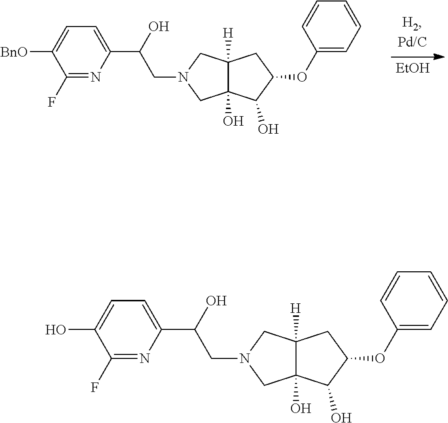 [SMILES: "EtOH" ; OC(CN1C[C@H]2C[C@H](Oc3ccccc3)[C@H](O)[C@@]2(O)C1)c1ccc(OCc2ccccc2)c(F)n1 ; Oc1ccc(C(O)CN2C[C@H]3C[C@H](Oc4ccccc4)[C@H](O)[C@@]3(O)C2)nc1F]

Example 3C: Using the same method, starting from Intermediate 260 (110 mg, 0.23 mmol), provided the title compound (60 mg).

LCMS: Rt 0.45 min; MS m/z 391.3 [M+H]⁺; Method E.

¹H NMR (400 MHz, DMSO-d₆) δ 6.50-6.41 (m, 4H), 6.13-6.10 (m, 3H), 3.91-3.84 (m, 2H), 3.14 (d, J=3.6 Hz, 1H), 2.17 (d, J=9.6 Hz, 1H), 1.99-1.88 (m, 3H), 1.81 (dd, J=9.6, 2.4 Hz, 1H), 1.64 (d, J=10.0 Hz, 1H), 1.60-1.53 (m, 1H), 1.46-1.40 (m, 1H), 0.81-0.75 (in, 1H).

Example 3D: Using the same method, starting from Intermediate 260 (105 mg, 0.21 mmol), provided the title compound (50 mg).

LCMS: Rt 1.25 min; MS m/z 390.8 [M+H]⁺; Method F.

¹H NMR (400 MHz, Methanol-d₄) δ 7.36-7.25 (m, 4H), 6.96-6.92 (m, 3H), 4.75-4.72 (m, 2H), 3.95 (d, J=3.6 Hz, 1H), 3.05 (d, J=10.4 Hz, 1H), 3.04-2.85 (m, 3H), 2.74 (dd, J=9.2, 2.8 Hz, 1H), 2.69-2.66 (m, 1H), 2.50-2.44 (m, 1H), 2.30-2.22 (m, 1H), 1.68-1.63 (m, 1H).

### Examples 4A, 4B, 4C and 4D

(3aS,4S,5S,6aR)-2-((S)-2-(6-chloro-5-hydroxypyridin-2-yl)-2-hydroxyethyl)-5-phenoxyhexahydrocyclopenta[c]pyrrole-3a,4(1H)-diol
(3aS,4S,5S,6aR)-2-((R)-2-(6-chloro-5-hydroxypyridin-2-yl)-2-hydroxyethyl)-5-phenoxyhexahydrocyclopenta[c]pyrrole-3a,4(1H)-diol
(3aR,4R,5R,6aS)-2-((S)-2-(6-chloro-5-hydroxypyridin-2-yl)-2-hydroxyethyl)-5-phenoxyhexahydrocyclopenta[c]pyrrole-3a,4(1H)-diol
(3aR,4R,5R,6aS)-2-((R)-2-(6-chloro-5-hydroxypyridin-2-yl)-2-hydroxyethyl)-5-phenoxyhexahydrocyclopenta[c]pyrrole-3a,4(1H)-diol

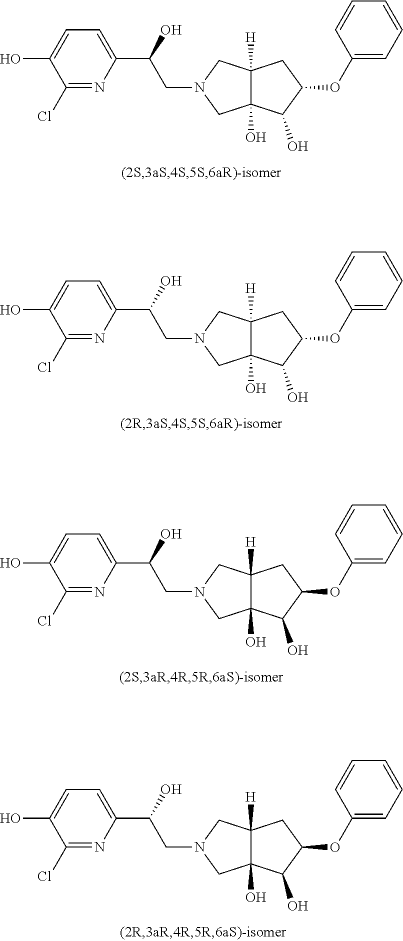 [SMILES: Oc1ccc([C@@H](O)CN2C[C@H]3C[C@H](Oc4ccccc4)[C@H](O)[C@@]3(O)C2)nc1Cl ; Oc1ccc([C@H](O)CN2C[C@H]3C[C@H](Oc4ccccc4)[C@H](O)[C@@]3(O)C2)nc1Cl ; Oc1ccc([C@@H](O)CN2C[C@@H]3C[C@@H](Oc4ccccc4)[C@@H](O)[C@]3(O)C2)nc1Cl ; Oc1ccc([C@H](O)CN2C[C@@H]3C[C@@H](Oc4ccccc4)[C@@H](O)[C@]3(O)C2)nc1Cl]

### Step 1 (Intermediates 27A and 27B): A Mixture of: (3aR,4R,5R,6aS)-2-((S)-2-(5-(benzyloxy)-6-chloropyridin-2-yl)-2-hydroxyethyl)-5-phenoxyhexahydrocyclopenta[c]pyrrole-3a,4(1H)-diol (3aR,4R,5R,6aS)-2-((R)-2-(5-(benzyloxy)-6-chloropyridin-2-yl)-2-hydroxyethyl)-5-phenoxyhexahydrocyclopenta[c]pyrrole-3a,4(1H)-diol

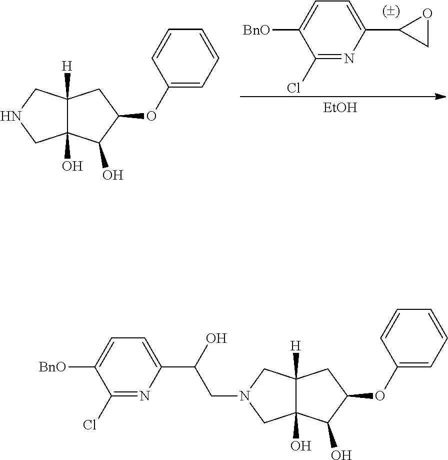 [SMILES: "EtOH" ; O[C@@H]1[C@H](Oc2ccccc2)C[C@H]2CNC[C@]21O ; Clc1nc(C2CO2)ccc1OCc1ccccc1 ; OC(CN1C[C@@H]2C[C@@H](Oc3ccccc3)[C@@H](O)[C@]2(O)C1)c1ccc(OCc2ccccc2)c(Cl)n1]

Using the same method as step 1 of Example 1A/1B/1C/1D, starting with Intermediate 6 (400 mg, 1.7 mmol) and Intermediate 18 (533 mg, 2.0 mmol), provided the title intermediates (450 mg).

LCMS: Rt 0.51 min; MS m/z 497.3 [M+H]⁺; Method D.

### Step 2 (Intermediates 27C and 27D): A Mixture of: (3aS,4S,5S,6aR)-2-((S)-2-(5-(benzyloxy)-6-chloropyridin-2-yl)-2-hydroxyethyl)-5-phenoxyhexahydrocyclopenta[c]pyrrole-3a,4(1H)-diol (3aS,4S,5S,6aR)-2-((R)-2-(5-(benzyloxy)-6-chloropyridin-2-yl)-2-hydroxyethyl)-5-phenoxyhexahydrocyclopenta[c]pyrrole-3a,4(1H)-diol

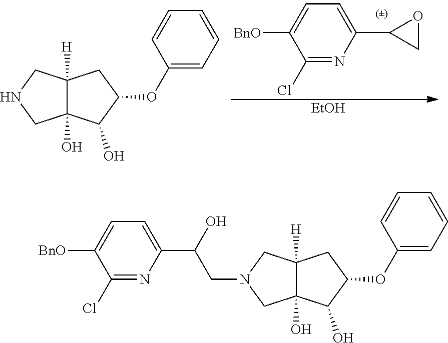

Using the same method as step 1, starting with Intermediate 5 (300 mg, 1.27 mmol) and Intermediate 18 (399 mg, 1.53 mmol), provided the title intermediates (350 mg).

LCMS: Rt 0.40 min; MS m/z 497.3 [M+H]⁺; Method D.

### Step 3 (Examples 4A and 4B)

(3aR,4R,5R,6aS)-2-((S)-2-(6-chloro-5-hydroxypyridin-2-yl)-2-hydroxyethyl)-5-phenoxyhexahydrocyclopenta[c]pyrrole-3a,4(1H)-diol
(3aR,4R,5R,6aS)-2-((R)-2-(6-chloro-5-hydroxypyridin-2-yl)-2-hydroxyethyl)-5-phenoxyhexahydrocyclopenta[c]pyrrole-3a,4(1H)-diol

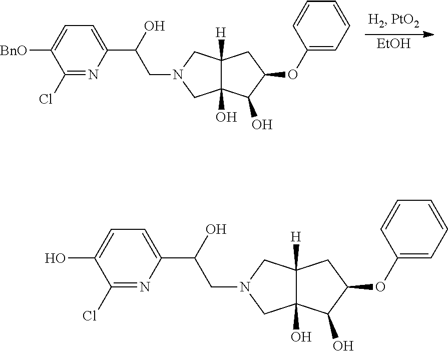 [SMILES: "EtOH" ; OC(CN1C[C@@H]2C[C@@H](Oc3ccccc3)[C@@H](O)[C@]2(O)C1)c1ccc(OCc2ccccc2)c(Cl)n1 ; Oc1ccc(C(O)CN2C[C@@H]3C[C@@H](Oc4ccccc4)[C@@H](O)[C@]3(O)C2)nc1Cl]

A solution of (3aR,4R,5R,6aS)-2-((S)-2-(5-(benzyloxy)-6-chloropyridin-2-yl)-2-hydroxyethyl)-5-phenoxyhexahydrocyclopenta[c]pyrrole-3a,4(1H)-diol and (3aR,4R,5R,6aS)-2-((R)-2-(5-(benzyloxy)-6-chloropyridin-2-yl)-2-hydroxyethyl)-5-phenoxyhexahydrocyclopenta[c]pyrrole-3a,4(1H)-diol (Intermediates 27A and 27B) (425 mg, 0.85 mmol) in EtOH (35 mL) was shaken with PtO₂ (43 mg) under a H₂ balloon for 18 h. The reaction was filtered through Celite, concentrated, and purified by the preparative HPLC method below to provide a mixture of Examples 4A and 4B (90 mg).

Column: Kinetics Evo C18, 21.2 mm×150 mm, Flow: 20 mL/min

Mobile phase: 0.02% NH₄OH in water (A), Acetonitrile (B)

LCMS: Rt 0.14 min; MS m/z 407.3 [M+H]⁺; Method D.

The two diastereomers were separated using the following chiral SFC method:

Column: Chiralpak IG (10 mm×250 mm, 5 micron), Flow: 10 mL/min

Mobile phase: CO₂ (A), 0.02% ammonia in IPA (B), Isocratic 60:40 (A:B)

Example 4A (chiral SFC Rt 7.01 min): 20 mg.

LCMS: Rt 0.11 min; MS m/z 407.2 [M+H]⁺; Method D.

¹H NMR (400 MHz, Methanol-d₄) δ 7.33-7.22 (m, 4H), 6.92-6.90 (m, 3H), 4.72-4.68 (m, 2H), 3.90 (d, J=3.6 Hz, 1H), 2.98 (d, J=9.6 Hz, 1H), 2.77-2.70 (m, 3H), 2.66-2.62 (m, 1H), 2.42 (d, J=9.6 Hz, 1H), 2.40-2.34 (m, 1H), 2.25-2.19 (m, 1H), 1.62-1.58 (m, 1H).

Example 4B (chiral SFC Rt 8.68 min): 22 mg.

LCMS: Rt 0.11 min; MS m/z 407.2 [M+H]⁺; Method D.

¹H NMR (400 MHz, Methanol-d₄) δ 7.33 (d, J=8.0 Hz, 1H), 7.27-7.23 (m, 3H), 6.93-6.90 (m, 3H), 4.72-4.68 (m, 2H), 3.94 (d, J=3.6 Hz, 1H), 2.98 (d, J=10.0 Hz, 1H), 2.79-2.70 (m, 3H), 2.66-2.62 (m, 1H), 2.46 (d, J=9.2 Hz, 1H), 2.51-2.46 (m, 1H), 2.26-2.18 (m, 1H), 1.61-1.57 (m, 1H).

### Step 4 (Examples 4C and 4D)

(3aS,4S,5S,6aR)-2-((S)-2-(6-chloro-5-hydroxypyridin-2-yl)-2-hydroxyethyl)-5-phenoxyhexahydrocyclopenta[c]pyrrole-3a,4(1H)-diol
(3aS,4S,5S,6aR)-2-((R)-2-(6-chloro-5-hydroxypyridin-2-yl)-2-hydroxyethyl)-5-phenoxyhexahydrocyclopenta[c]pyrrole-3a,4(1H)-diol

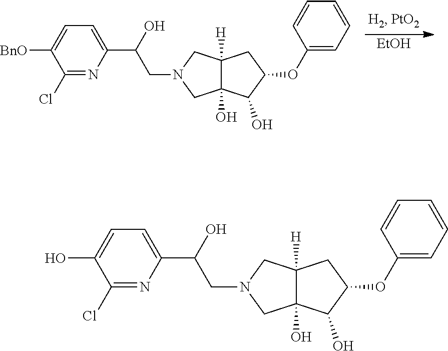 [SMILES: "EtOH" ; OC(CN1C[C@H]2C[C@H](Oc3ccccc3)[C@H](O)[C@@]2(O)C1)c1ccc(OCc2ccccc2)c(Cl)n1 ; Oc1ccc(C(O)CN2C[C@H]3C[C@H](Oc4ccccc4)[C@H](O)[C@@]3(O)C2)nc1Cl]

Using the same method as step 3, starting with Intermediates 27C and 27D (350 mg, 0.70 mmol) and purifying with the preparative HPLC method below, provided a mixture of Examples 4C and 4D (120 mg).

Column: Kinetics Evo C18 21.2 mm×150 mm, Flow: 20 mL/min

Mobile phase: 0.1% TFA in water (A), Acetonitrile (B)

LCMS: Rt 0.14 min; MS m/z 407.2 [M+H]⁺; Method D.

The two diastereomers were separated using the following chiral HPLC method:

Column: Chiralpak IA, Flow: 10 mL/min

Mobile phase: Hexane (A), 0.1% DEA in MeOH:IPA 25:75 (B), Isocratic 85:15 A:B

Example 4C (chiral HPLC Rt 7.14 min).

This compound was further purified by the following chiral SFC method, giving 13 mg of product.

Column: Chiralpak IG (10 mm×250 mm, 5 micron), Flow: 10 mL/min

Mobile phase: CO₂ (A), 0.02% NH₄OH in IPA:EtOH:MeOH (80:10:10) (B), Isocratic 75:25 (A:B)

LCMS: Rt 0.12 min; MS m/z 407.2 [M+H]⁺; Method D.

¹H NMR (400 MHz, Methanol-d₄) δ 7.33-7.18 (m, 4H), 6.95-6.89 (m, 3H), 4.75-4.68 (m, 2H), 3.95 (d, J=4.0 Hz, 1H), 2.98 (d, J=9.6 Hz, 1H), 2.79-2.68 (m, 3H), 2.66-2.59 (m, 1H), 2.46 (d, J=9.6 Hz, 1H), 2.42-2.35 (m, 1H), 2.28-2.19 (m, 1H), 1.64-1.56 (m, 1H).

Example 4D (chiral HPLC Rt 9.02 min): 35 mg.

LCMS: Rt 0.12 min; MS m/z 407.2 [M+H]⁺; Method D.

¹H NMR (400 MHz, Methanol-d₄) δ 7.31-7.16 (m, 4H), 6.94-6.88 (m, 3H), 4.73-4.66 (m, 2H), 3.90 (d, J=4.0 Hz, 1H), 3.00-2.95 (m, 1H), 2.77-2.68 (m, 3H), 2.66-2.60 (m, 1H), 2.43-2.33 (m, 2H), 2.27-2.18 (m, 1H), 1.63-1.57 (m, 1H).

### Examples 5A, 5B, 5C and 5D

(3aS,4S,5S,6aR)-2-((R)-2-(3,5-difluoro-4-hydroxyphenyl)-2-hydroxyethyl)-5-phenoxyhexahydrocyclopenta[c]pyrrole-3a,4(1H)-diol
(3aS,4S,5S,6aR)-2-((S)-2-(3,5-difluoro-4-hydroxyphenyl)-2-hydroxyethyl)-5-phenoxyhexahydrocyclopenta[c]pyrrole-3a,4(1H)-diol
(3aR,4R,5R,6aS)-2-((R)-2-(3,5-difluoro-4-hydroxyphenyl)-2-hydroxyethyl)-5-phenoxyhexahydrocyclopenta[c]pyrrole-3a,4(1H)-diol
(3aR,4R,5R,6aS)-2-((S)-2-(3,5-difluoro-4-hydroxyphenyl)-2-hydroxyethyl)-5-phenoxyhexahydrocyclopenta[c]pyrrole-3a,4(1H)-diol

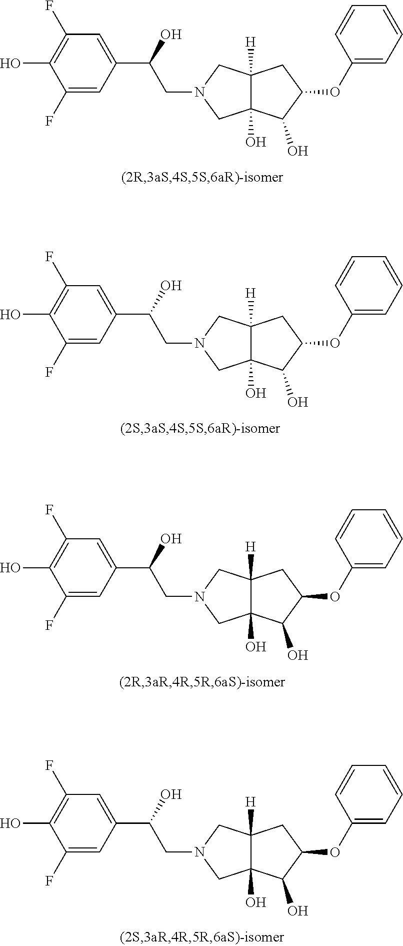 [SMILES: Oc1c(F)cc([C@@H](O)CN2C[C@H]3C[C@H](Oc4ccccc4)[C@H](O)[C@@]3(O)C2)cc1F ; Oc1c(F)cc([C@H](O)CN2C[C@H]3C[C@H](Oc4ccccc4)[C@H](O)[C@@]3(O)C2)cc1F ; Oc1c(F)cc([C@@H](O)CN2C[C@@H]3C[C@@H](Oc4ccccc4)[C@@H](O)[C@]3(O)C2)cc1F ; Oc1c(F)cc([C@H](O)CN2C[C@@H]3C[C@@H](Oc4ccccc4)[C@@H](O)[C@]3(O)C2)cc1F]

### Step 1 (Intermediates 28A and 28B): A Mixture of: (3aS,4S,5S,6aR)-2-((R)-2-(4-(benzyloxy)-3,5-difluorophenyl)-2-hydroxyethyl)-5-phenoxyhexahydrocyclopenta[c]pyrrole-3a,4(1H)-diol (3aS,4S,5S,6aR)-2-((S)-2-(4-(benzyloxy)-3,5-difluorophenyl)-2-hydroxyethyl)-5-phenoxyhexahydrocyclopenta[c]pyrrole-3a,4(1H)-diol

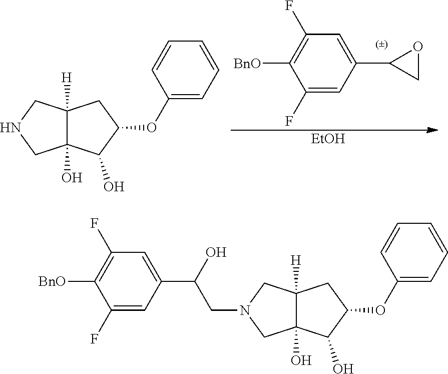

Using the same method as step 1 of Examples 1A/1B/1C/1D, starting with Intermediate 5 (600 mg, 2.55 mmol) and Intermediate 20 (801 mg, 3.06 mmol), provided the title intermediates (600 mg).

LCMS: Rt 1.36 min; MS m/z 497.8 [M+H]⁺; Method F.

### Step 2 (Intermediates 28C and 28D): A Mixture of: (3aR,4R,5R,6aS)-2-((R)-2-(4-(benzyloxy)-3,5-difluorophenyl)-2-hydroxyethyl)-5-phenoxyhexahydrocyclopenta[c]pyrrole-3a,4(1H)-diol (3aR,4R,5R,6aS)-2-((S)-2-(4-(benzyloxy)-3,5-difluorophenyl)-2-hydroxyethyl)-5-phenoxyhexahydrocyclopenta[c]pyrrole-3a,4(1H)-diol

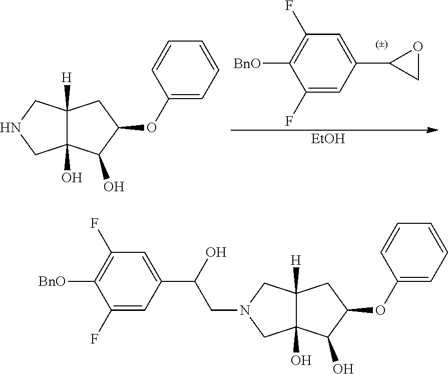

Using the same method as step 1, starting with Intermediate 6 (600 mg, 2.55 mmol) and Intermediate 20 (801 mg, 3.06 mmol), provided the title intermediates (450 mg).

LCMS: Rt 1.91 min; MS m/z 498.1 [M+H]⁺; Method E.

### Step 3 (Examples 5A and 5B)

(3aS,4S,5S,6aR)-2-((R)-2-(3,5-difluoro-4-hydroxyphenyl)-2-hydroxyethyl)-5-phenoxyhexahydrocyclopenta[c]pyrrole-3a,4(1H)-diol
(3aS,4S,5S,6aR)-2-((S)-2-(3,5-difluoro-4-hydroxyphenyl)-2-hydroxyethyl)-5-phenoxyhexahydrocyclopenta[c]pyrrole-3a,4(1H)-diol

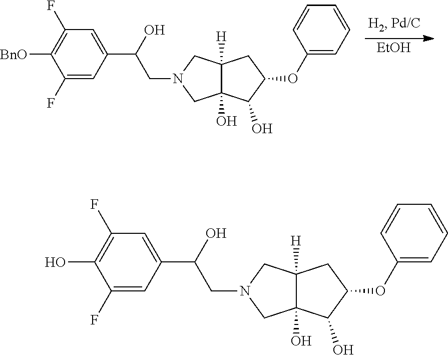 [SMILES: "EtOH" ; OC(CN1C[C@H]2C[C@H](Oc3ccccc3)[C@H](O)[C@@]2(O)C1)c1cc(F)c(OCc2ccccc2)c(F)c1 ; Oc1c(F)cc(C(O)CN2C[C@H]3C[C@H](Oc4ccccc4)[C@H](O)[C@@]3(O)C2)cc1F]

Using the same method as step 3 of Examples 2A/2B, starting with Intermediates 28A and 28B (600 mg, 1.20 mmol), provided a mixture of Examples 5A and 5B (450 mg).

LCMS: Rt 0.47 min; MS m/z 408.2 [M+H]⁺; Method E.

The two diastereomers were separated using the following chiral SFC method:

Column: Chiralpak IG (10 mm×250 mm, 5 micron), Flow: 17 mL/min

Mobile phase: CO₂ (A), 0.1% DEA in EtOH:MeOH 1:1 (B), Isocratic 75:25 (A:B)

Example 5A (chiral SFC Rt 8.55 min): 70 mg.

LCMS: Rt 0.14 min; MS m/z 408.1 [M+H]⁺; Method D.

¹H NMR (400 MHz, Methanol-d₄) δ 7.29-7.25 (m, 2H), 6.95-6.91 (m, 5H), 4.77-4.72 (m, 1H), 4.68-4.64 (m, 1H), 3.97 (d, J=3.6 Hz, 1H), 2.97 (d, J=10.0 Hz, 1H), 2.73-2.67 (m, 2H), 2.63-2.58 (m, 2H), 2.45 (d, J=9.6 Hz, 1H), 2.42-2.38 (m, 1H), 2.29-2.22 (m, 1H), 1.63-1.58 (m, 1H).

Example 5B (chiral SFC Rt 11.14 min).

This compound was further purified by the following preparative HPLC method, providing 55 mg.

Column: Gemini-NX (150 mm×21.2 mm), 5.0 μm, Flow: 20 mL/min

Mobile phase: 0.1% TFA in water (A), Acetonitrile (B), Isocratic 75:25 (A:B)

LCMS: Rt 0.14 min; MS m/z 408.3 [M+H]⁺; Method D.

¹H NMR (400 MHz, Methanol-d₄) δ 7.29-7.25 (m, 2H), 6.97-6.91 (m, 5H), 4.77-4.72 (m, 1H), 4.69-4.66 (m, 1H), 3.94 (d, J=3.6 Hz, 1H), 2.96 (d, J=9.6 Hz, 1H), 2.76-2.59 (m, 4H), 2.41 (d, J=9.6 Hz, 2H), 2.30-2.22 (m, 1H), 1.67-1.61 (m, 1H).

### Step 4 (Examples 5C and 5D)

(3aR,4R,5R,6aS)-2-((R)-2-(3,5-difluoro-4-hydroxyphenyl)-2-hydroxyethyl)-5-phenoxyhexahydrocyclopenta[c]pyrrole-3a,4(1H)-diol
(3aR,4R,5R,6aS)-2-((S)-2-(3,5-difluoro-4-hydroxyphenyl)-2-hydroxyethyl)-5-phenoxyhexahydrocyclopenta[c]pyrrole-3a,4(1H)-diol

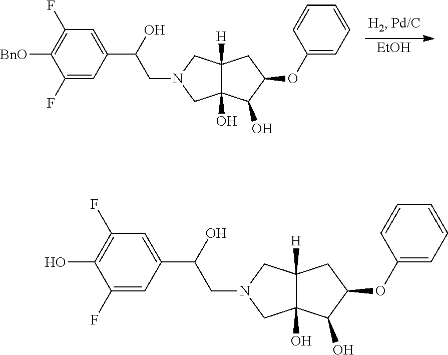 [SMILES: "EtOH" ; OC(CN1C[C@@H]2C[C@@H](Oc3ccccc3)[C@@H](O)[C@]2(O)C1)c1cc(F)c(OCc2ccccc2)c(F)c1 ; Oc1c(F)cc(C(O)CN2C[C@@H]3C[C@@H](Oc4ccccc4)[C@@H](O)[C@]3(O)C2)cc1F]

Using the same method as step 3, starting with Intermediates 28C and 28D (450 mg, 0.90 mmol) and purifying with the preparative HPLC method below, provided a mixture of Examples 5C and 5D (250 mg).

Column: Kinetex, Flow: 20 mL/min

Mobile phase: 0.1% Formic acid in water (A), Acetonitrile (B)

LCMS: Rt 0.14 min; MS m/z 408.1 [M+H]⁺; Method D.

The two diastereomers were separated using the following chiral SFC method:

Column: Chiralpak IG (10 mm×250 mm, 5 micron), Flow: 13 mL/min

Mobile phase: CO₂ (A), 0.02% NH₄OH in IPA (B), Isocratic 60:40 (A:B)

Example 5C (chiral SFC Rt 8.55 min): 65 mg.

LCMS: Rt 0.14 min; MS m/z 408.2 [M+H]⁺; Method D.

¹H NMR (400 MHz, Methanol-d₄) δ 7.29-7.25 (m, 2H), 6.97-6.91 (m, 5H), 4.77-4.72 (m, 1H), 4.69-4.66 (m, 1H), 3.94 (d, J=3.6 Hz, 1H), 2.96 (d, J=9.6 Hz, 1H), 2.76-2.59 (m, 4H), 2.41 (d, J=10.0 Hz, 2H), 2.29-2.25 (m, 1H), 1.67-1.61 (m, 1H).

Example 5D (chiral SFC Rt 11.54 min): 65 mg.

LCMS: Rt 0.14 min; MS m/z 408.2 [M+H]⁺; Method D.

¹H NMR (400 MHz, Methanol-d₄) δ 7.29-7.25 (m, 2H), 6.97-6.91 (m, 5H), 4.77-4.72 (m, 1H), 4.68-4.65 (m, 1H), 3.96 (d, J=3.6 Hz, 1H), 2.97 (d, J=9.2 Hz, 1H), 2.73-2.68 (m, 2H), 2.63-2.59 (m, 2H), 2.46 (d, J=10.0 Hz, 1H), 2.42-2.38 (m, 1H), 2.29-2.22 (m, 1H), 1.65-1.59 (m, 1H).

### Examples 6A, 6B, 6C and 6D

(3aS,4S,5S,6aR)-2-((R)-2-(3,5-difluoro-4-hydroxyphenyl)-2-hydroxyethyl)-5-(2-fluorophenoxy)hexahydrocyclopenta[c]pyrrole-3a,4(1H)-diol
(3aS,4S,5S,6aR)-2-((S)-2-(3,5-difluoro-4-hydroxyphenyl)-2-hydroxyethyl)-5-(2-fluorophenoxy)hexahydrocyclopenta[c]pyrrole-3a,4(1H)-diol
(3aR,4R,5R,6aS)-2-((R)-2-(3,5-difluoro-4-hydroxyphenyl)-2-hydroxyethyl)-5-(2-fluorophenoxy)hexahydrocyclopenta[c]pyrrole-3a,4(1H)-diol
(3aR,4R,5R,6aS)-2-((S)-2-(3,5-difluoro-4-hydroxyphenyl)-2-hydroxyethyl)-5-(2-fluorophenoxy)hexahydrocyclopenta[c]pyrrole-3a,4(1H)-diol

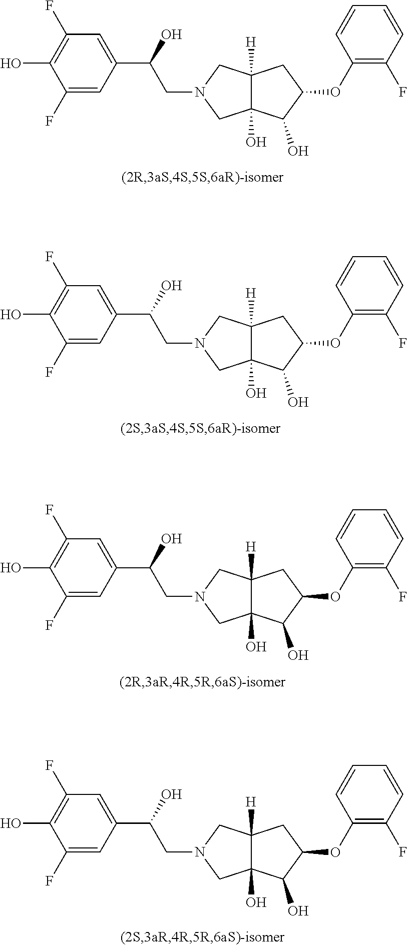 [SMILES: Oc1c(F)cc([C@@H](O)CN2C[C@H]3C[C@H](Oc4ccccc4F)[C@H](O)[C@@]3(O)C2)cc1F ; Oc1c(F)cc([C@H](O)CN2C[C@H]3C[C@H](Oc4ccccc4F)[C@H](O)[C@@]3(O)C2)cc1F ; Oc1c(F)cc([C@@H](O)CN2C[C@@H]3C[C@@H](Oc4ccccc4F)[C@@H](O)[C@]3(O)C2)cc1F ; Oc1c(F)cc([C@H](O)CN2C[C@@H]3C[C@@H](Oc4ccccc4F)[C@@H](O)[C@]3(O)C2)cc1F]

### Step 1: A Mixture of: (3aS,4S,5S,6aR)-2-((R)-2-(4-(benzyloxy)-3,5-difluorophenyl)-2-hydroxyethyl)-5-(2-fluorophenoxy)hexahydrocyclopenta[c]pyrrole-3a,4(1H)-diol (3aS,4S,5S,6aR)-2-((S)-2-(4-(benzyloxy)-3,5-difluorophenyl)-2-hydroxyethyl)-5-(2-fluorophenoxy)hexahydrocyclopenta[c]pyrrole-3a,4(1H)-diol (3aR,4R,5R,6aS)-2-((R)-2-(4-(benzyloxy)-3,5-difluorophenyl)-2-hydroxyethyl)-5-(2-fluorophenoxy)hexahydrocyclopenta[c]pyrrole-3a,4(1H)-diol (3aR,4R,5R,6aS)-2-((S)-2-(4-(benzyloxy)-3,5-difluorophenyl)-2-hydroxyethyl)-5-(2-fluorophenoxy)hexahydrocyclopenta[c]pyrrole-3a,4(1H)-diol

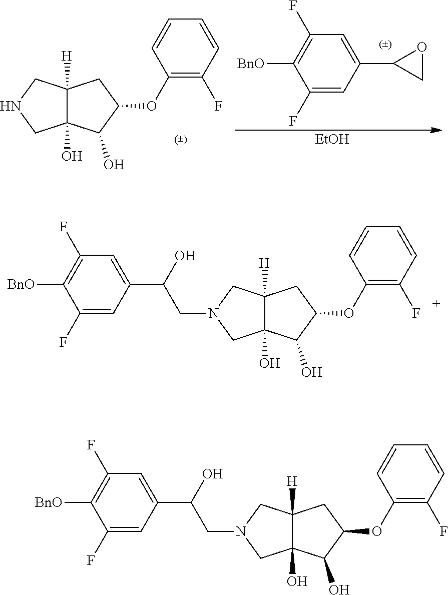

Using the same method as step 1 of Examples 1A/1B/1C/1D, starting from Intermediate 7 (130 mg, 0.52 mmol) and Intermediate 20 (163 mg, 0.62 mmol), provided the title intermediates (140 mg).

LCMS: Rt 0.69 min; MS m/z 515.9 [M+H]⁺; Method D.

### Step 2: A Mixture of Examples 6A, 6B, 6C, and 6D

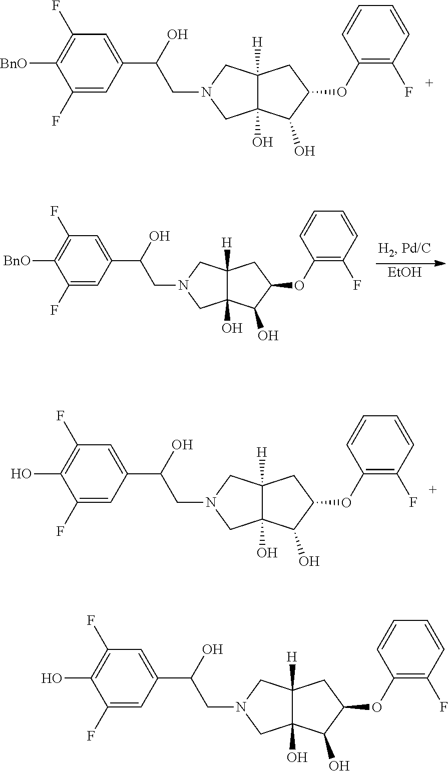 [SMILES: "EtOH" ; OC(CN1C[C@H]2C[C@H](Oc3ccccc3F)[C@H](O)[C@@]2(O)C1)c1cc(F)c(OCc2ccccc2)c(F)c1 ; OC(CN1C[C@@H]2C[C@@H](Oc3ccccc3F)[C@@H](O)[C@]2(O)C1)c1cc(F)c(OCc2ccccc2)c(F)c1 ; Oc1c(F)cc(C(O)CN2C[C@H]3C[C@H](Oc4ccccc4F)[C@H](O)[C@@]3(O)C2)cc1F ; Oc1c(F)cc(C(O)CN2C[C@@H]3C[C@@H](Oc4ccccc4F)[C@@H](O)[C@]3(O)C2)cc1F]

Using the same method as step 3 of Examples 2A/2B, starting with the mixture of intermediates from the previous step (140 mg, 0.27 mmol), provided a mixture of Examples 6A, 6B, 6C and 6D (80 mg).

LCMS: Rt 1.24 min; MS m/z 426.1 [M+H]⁺; Method E.

Step 3: Chiral separation of Examples 6A, 6B, 6C and 6D

The mixture was separated using the following chiral HPLC method:

Column: Chiralpak IA, Flow: 7 mL/min

Mobile phase: Acetonitrile (A), IPA (B), Isocratic 90:10 (A:B)

This method separated example 6A from the other three isomers, which eluted together.

Example 6A (chiral HPLC Rt 17.01 min): 15 mg.

LCMS: Rt 0.12 min; MS m/z 426.1 [M+H]⁺; Method D.

¹H NMR (400 MHz, Methanol-d₄) δ 7.11-7.05 (m, 3H), 6.96-6.91 (m, 3H), 4.77-4.73 (m, 1H), 4.69-4.66 (m, 1H), 3.97 (d, J=3.6 Hz, 1H), 3.00 (d, J=9.6 Hz, 1H), 2.78-2.70 (m, 2H), 2.65 (d, J=7.2 Hz, 2H), 2.60-2.43 (m, 2H), 2.30-2.15 (m, 1H), 1.60-1.55 (m, 1H).

The remaining mixture was separated using the following chiral HPLC method:

Column: Chiralpak IA, Flow: 7 mL/min

Mobile phase: Hexane (A), EtOH (B), Isocratic 85:15 (A:B)

This method separated example 6B from the other two isomers, which eluted together.

Example 6B (chiral HPLC Rt 5.24 min): 15 mg.

LCMS: Rt 0.13 min; MS m/z 426.0 [M+H]⁺; Method D.

¹H NMR (400 MHz, Methanol-d₄) δ 7.10-7.06 (m, 3H), 6.95-6.92 (m, 3H), 4.77-4.72 (m, 1H), 4.68-4.62 (m, 1H), 3.96 (d, J=3.2 Hz, 1H), 2.98 (d, J=10.0 Hz, 1H), 2.76-2.59 (m, 4H), 2.48-2.40 (m, 2H), 2.30-2.20 (m, 1H), 1.60-1.53 (m, 1H).

The remaining mixture was separated using the following chiral SFC method:

Column: Chiralpak IG (10 mm×250 mm, 5 micron), Flow: 14 mL/min

Mobile phase: CO₂ (A), EtOH:MeOH 1:1 (B), Isocratic 65:35 (A:B)

Example 6C (chiral SFC Rt 6.83 min): 10 mg.

LCMS: Rt 0.61 min; MS m/z 426.0 [M+H]⁺; Method E.

¹H NMR (400 MHz, Methanol-d₄) δ 7.10-7.05 (m, 3H), 6.95-6.90 (m, 3H), 4.76-4.72 (m, 1H), 4.65-4.62 (m, 1H), 3.93 (d, J=3.6 Hz, 1H), 2.95 (d, J=9.6 Hz, 1H), 2.70-2.54 (m, 4H), 2.45-2.39 (m, 1H), 2.35 (d, J=9.6 Hz, 1H), 2.30-2.20 (m, 1H), 1.61-1.55 (m, 1H).

Example 6D (chiral SFC Rt 9.41 min): 10 mg.

LCMS: Rt 1.28 min; MS m/z 426.0 [M+H]⁺; Method F.

¹H NMR (400 MHz, Methanol-d₄) δ 7.10-7.05 (m, 3H), 6.96-6.90 (m, 3H), 4.76-4.72 (m, 1H), 4.65-4.62 (m, 1H), 3.93 (d, J=3.6 Hz, 1H), 2.95 (d, J=10.0 Hz, 1H), 2.72-2.54 (m, 4H), 2.45-2.39 (m, 1H), 2.35 (d, J=9.6 Hz, 1H), 2.29-2.22 (m, 1H), 1.61-1.55 (m, 1H).

### Examples 7A, 7B, 7C and 7D

(3aS,5S,6aR)-2-((S)-2-hydroxy-2-(5-hydroxypyridin-2-yl)ethyl)-5-phenoxyhexahydrocyclopenta[c]pyrrol-3a(1H)-ol
(3aS,5S,6aR)-2-((R)-2-hydroxy-2-(5-hydroxypyridin-2-yl)ethyl)-5-phenoxyhexahydrocyclopenta[c]pyrrol-3a(1H)-ol
(3aR,5R,6aS)-2-((S)-2-hydroxy-2-(5-hydroxypyridin-2-yl)ethyl)-5-phenoxyhexahydrocyclopenta[c]pyrrol-3a(1H)-ol
(3aR,5R,6aS)-2-((R)-2-hydroxy-2-(5-hydroxypyridin-2-yl)ethyl)-5-phenoxyhexahydrocyclopenta[c]pyrrol-3a(1H)-ol

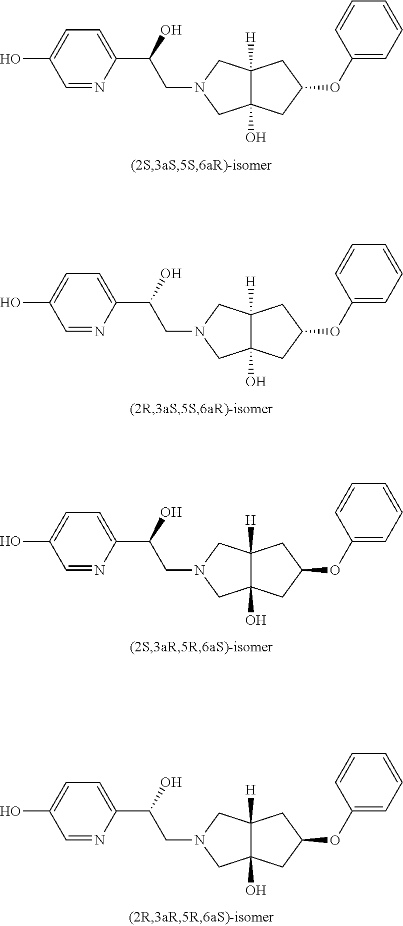 [SMILES: Oc1ccc([C@@H](O)CN2C[C@H]3C[C@H](Oc4ccccc4)C[C@@]3(O)C2)nc1 ; Oc1ccc([C@H](O)CN2C[C@H]3C[C@H](Oc4ccccc4)C[C@@]3(O)C2)nc1 ; Oc1ccc([C@@H](O)CN2C[C@@H]3C[C@@H](Oc4ccccc4)C[C@]3(O)C2)nc1 ; Oc1ccc([C@H](O)CN2C[C@@H]3C[C@@H](Oc4ccccc4)C[C@]3(O)C2)nc1]

Using the same methods as Examples 6A/6B/6C/6D, starting from Intermediate 1 and Intermediate 14, provided a mixture of Examples 7A/7B/7C/7D. The mixture was separated using the following chiral SFC method:

Column: Chiralpak ID 21×250 mm, Flow rate: 80 g per minute

Mobile phase: CO₂ (A), MeOH with 10 mM NH₄OH (B), Isocratic 65:35 (A:B)

This gave first a mixture of examples 7A and 7B (Rt=2.1 min), followed by example 7C (Rt=3.1 min), then example 7D (Rt=5.4 min).

Example 7C: 11 mg.

Analytical chiral SFC: Rt 3.29 min (Column: Chiralpak ID 4.6×100 mm, 5 μm, flow rate 5 mL/min, mobile phase A: CO₂, phase B: methanol with 10 mM NH₄OH, gradient: 5-55% B).

LCMS: Rt 0.41 min; MS m/z 357.1 [M+H]⁺; Method C.

¹H NMR (400 MHz, Methanol-d₄) δ 8.07-7.95 (m, 1H), 7.41 (d, J=8.6 Hz, 1H), 7.31-7.17 (m, 3H), 6.96-6.79 (m, 3H), 4.81-4.70 (m, 2H), 2.93-2.61 (m, 5H), 2.57-2.36 (m, 2H), 2.26 (dd, J=13.3, 5.2 Hz, 1H), 2.22-1.93 (m, 2H), 1.80 (dt, J=12.6, 5.1 Hz, 1H).

Example 7D: 7.6 mg.

Analytical chiral SFC: Rt 3.79 min (Column: Chiralpak ID 4.6×100 mm, 5 μm, flow rate 5 mL/min, mobile phase A: CO₂, phase B: methanol with 10 mM NH₄OH, gradient: 5-55% B).

LCMS: Rt 0.41 min; MS m/z 357.0 [M+H]⁺; Method C.

¹H NMR (400 MHz, Methanol-d₄) δ 8.02 (d, J=2.8 Hz, 1H), 7.40 (d, J=8.5 Hz, 1H), 7.30-7.18 (m, 3H), 6.94-6.80 (m, 3H), 4.82-4.71 (m, 2H), 2.95-2.86 (m, 1H), 2.82 (d, J=9.5 Hz, 1H), 2.78-2.67 (m, 2H), 2.57 (d, J=9.4 Hz, 1H), 2.52-2.40 (m, 2H), 2.26-2.10 (m, 2H), 2.00 (dd, J=13.4, 5.9 Hz, 1H), 1.82 (dt, J=12.8, 5.1 Hz, 1H).

Examples 7A and 7B were separated using the following chiral SFC method:

Column: Chiralpak IG 21×250 mm, Flow rate: 80 g per minute

Mobile phase: CO₂ (A), IPA with 10 mM NH₄OH (B), Isocratic 65:35 (A:B)

Example 7A (preparative chiral SFC Rt 2.3 min): 8 mg.

Analytical chiral SFC: Rt 3.07 min (Column: Chiralpak IG 4.6×100 mm, 5 μm, flow rate 5 mL/min, mobile phase A: CO₂, phase B: IPA with 10 mM NH₄OH, gradient: 5-55% B).

LCMS: Rt 0.42 min; MS m/z 357.1 [M+H]⁺; Method C.

¹H NMR (400 MHz, Methanol-d₄) δ 8.02 (dd, J=2.8, 0.7 Hz, 1H), 7.40 (d, J=8.6 Hz, 1H), 7.30-7.18 (m, 3H), 6.92-6.82 (m, 3H), 4.82-4.70 (m, 2H), 2.95-2.87 (m, 1H), 2.82 (d, J=9.4 Hz, 1H), 2.78-2.66 (m, 2H), 2.57 (d, J=9.4 Hz, 1H), 2.53-2.40 (m, 2H), 2.27-2.09 (m, 2H), 2.00 (ddd, J=13.4, 6.0, 1.4 Hz, 1H), 1.88-1.77 (m, 1H).

Example 7B (preparative chiral SFC Rt 3.9 min): 9.4 mg.

Analytical chiral SFC: Rt 3.42 min (Column: Chiralpak IG 4.6×100 mm, 5 μm, flow rate 5 mL/min, mobile phase A: CO₂, phase B: IPA with 10 mM NH₄OH, gradient: 5-55% B).

LCMS: Rt 0.45 min; MS m/z 357.3 [M+H]⁺; Method C.

¹H NMR (400 MHz, Methanol-d₄) δ 8.03 (dd, J=2.8, 0.6 Hz, 1H), 7.41 (d, J=8.5 Hz, 1H), 7.30-7.18 (m, 3H), 6.94-6.81 (m, 3H), 4.83-4.72 (m, 2H), 3.00-2.89 (m, 1H), 2.89-2.68 (m, 4H), 2.60-2.43 (m, 2H), 2.28 (dd, J=13.4, 5.3 Hz, 1H), 2.22-2.10 (m, 1H), 2.07-1.97 (m, 1H), 1.88-1.77 (m, 1H).

### Examples 8A, 8B, 8C and 8D

(3aS,5S,6aR)-2-((R)-2-(3,5-difluoro-4-hydroxyphenyl)-2-hydroxyethyl)-5-(2-fluorophenoxy)hexahydrocyclopenta[c]pyrrol-3a(1H)-ol
(3aS,5S,6aR)-2-((S)-2-(3,5-difluoro-4-hydroxyphenyl)-2-hydroxyethyl)-5-(2-fluorophenoxy)hexahydrocyclopenta[c]pyrrol-3a(1H)-ol
(3aR,5R,6aS)-2-((R)-2-(3,5-difluoro-4-hydroxyphenyl)-2-hydroxyethyl)-5-(2-fluorophenoxy)hexahydrocyclopenta[c]pyrrol-3a(1H)-ol
(3aR,5R,6aS)-2-((S)-2-(3,5-difluoro-4-hydroxyphenyl)-2-hydroxyethyl)-5-(2-fluorophenoxy)hexahydrocyclopenta[c]pyrrol-3a(1H)-ol

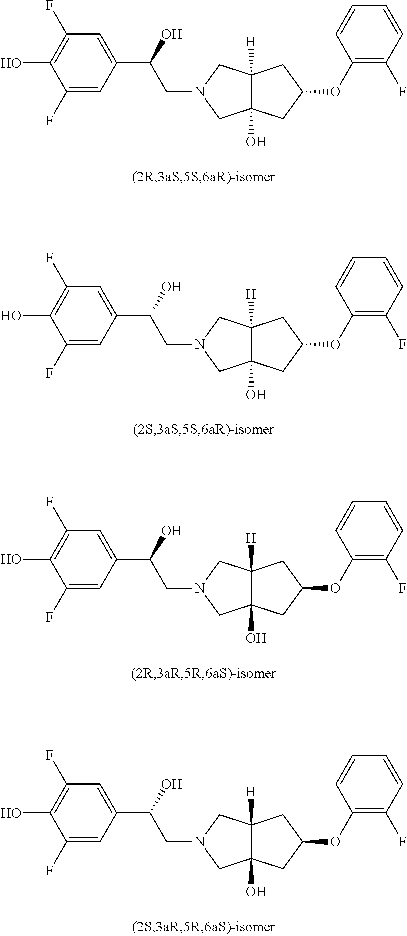 [SMILES: Oc1c(F)cc([C@@H](O)CN2C[C@H]3C[C@H](Oc4ccccc4F)C[C@@]3(O)C2)cc1F ; Oc1c(F)cc([C@H](O)CN2C[C@H]3C[C@H](Oc4ccccc4F)C[C@@]3(O)C2)cc1F ; Oc1c(F)cc([C@@H](O)CN2C[C@@H]3C[C@@H](Oc4ccccc4F)C[C@]3(O)C2)cc1F ; Oc1c(F)cc([C@H](O)CN2C[C@@H]3C[C@@H](Oc4ccccc4F)C[C@]3(O)C2)cc1F]

Using the same methods as Examples 6A/6B/6C/6D, starting from Intermediate 3 and Intermediate 20, provided a mixture of Examples 8A/8B/8C/8D. The mixture was separated using the following chiral HPLC method:

Column: Chiralpak IA (20 mm×250 mm, 5 micron), Flow: 18 mL/min

Mobile phase: Hexane (A), EtOH (B), Isocratic: 70:30 (A:B)

Example 8A (chiral HPLC Rt 3.31 min): 32 mg.

LCMS: Rt 0.17 min; MS m/z 410.0 [M+H]⁺; Method D.

¹H NMR (400 MHz, Methanol-d₄) δ 7.09-7.00 (m, 3H), 6.94-6.88 (m, 3H), 4.87-4.81 (m, 1H), 4.68-4.62 (m, 1H), 2.90 (t, J=9.6 Hz, 1H), 2.80 (d, J=9.6 Hz, 1H), 2.70-2.63 (m, 1H), 2.58-2.46 (m, 4H), 2.28-2.18 (m, 2H), 2.06 (dd, J=13.6, 6.0 Hz, 1H), 1.87-1.81 (m, 1H).

Example 8B (chiral HPLC Rt 3.02 min): 18 mg.

LCMS: Rt 0.17 min; MS m/z 410.0 [M+H]⁺; Method D.

¹H NMR (400 MHz, Methanol-d₄) δ 7.09-7.04 (m, 3H), 6.96-6.90 (m, 3H), 4.87-4.83 (m, 1H), 4.70-4.67 (m, 1H), 3.02-2.97 (m, 1H), 2.87 (d, J=9.6 Hz, 1H), 2.79-2.74 (m, 2H), 2.66-2.62 (m, 1H), 2.58-2.52 (m, 2H), 2.30 (dd, J=13.2, 5.2 Hz, 1H), 2.25-2.18 (m, 1H), 2.08 (dd, J=13.6, 5.6 Hz, 1H), 1.87-1.81 (m, 1H).

Example 8C (chiral HPLC Rt 4.16 min): 41 mg.

LCMS: Rt 0.17 min; MS m/z 410.0 [M+H]⁺; Method D.

¹H NMR (400 MHz, Methanol-d₄) δ 7.09-7.01 (m, 3H), 6.95-6.88 (m, 3H), 4.87-4.81 (m, 1H), 4.68-4.62 (m, 1H), 2.90-2.80 (m, 2H), 2.72-2.65 (m, 2H), 2.58-2.48 (m, 3H), 2.31-2.17 (m, 2H), 2.10-2.03 (m, 1H), 1.85-1.79 (m, 1H).

Example 8D (chiral HPLC Rt 6.48 min): 44 mg.

LCMS: Rt 0.17 min; MS m/z 409.9 [M+H]⁺; Method D.

¹H NMR (400 MHz, Methanol-d₄) δ 7.08-7.01 (m, 3H), 6.94-6.89 (m, 3H), 4.85-4.81 (m, 1H), 4.67-4.64 (m, 1H), 2.95-2.90 (m, 1H), 2.82 (d, J=9.2 Hz, 1H), 2.70-2.65 (m, 1H), 2.60-2.55 (m, 2H), 2.52-2.48 (m, 2H), 2.28-2.18 (m, 2H), 2.06 (dd, J=13.6, 6.0 Hz, 1H), 1.87-1.81 (m, 1H).

### Examples 9A, 9B, 9C and 9D

(3aS,5S,6aR)-2-((S)-2-(6-fluoro-5-hydroxypyridin-2-yl)-2-hydroxyethyl)-5-phenoxyhexahydrocyclopenta[c]pyrrol-3a(1H)-ol
(3aS,5S,6aR)-2-((R)-2-(6-fluoro-5-hydroxypyridin-2-yl)-2-hydroxyethyl)-5-phenoxyhexahydrocyclopenta[c]pyrrol-3a(1H)-ol
(3aR,5R,6aS)-2-((S)-2-(6-fluoro-5-hydroxypyridin-2-yl)-2-hydroxyethyl)-5-phenoxyhexahydrocyclopenta[c]pyrrol-3a(1H)-ol
(3aR,5R,6aS)-2-((R)-2-(6-fluoro-5-hydroxypyridin-2-yl)-2-hydroxyethyl)-5-phenoxyhexahydrocyclopenta[c]pyrrol-3a(1H)-ol

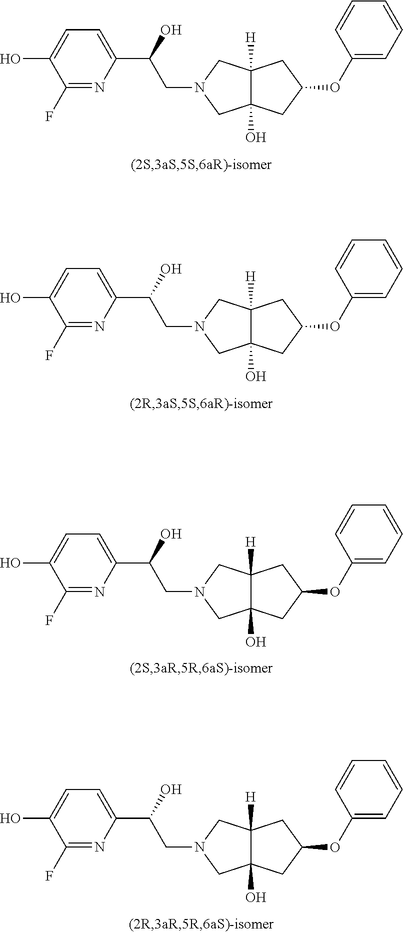 [SMILES: Oc1ccc([C@@H](O)CN2C[C@H]3C[C@H](Oc4ccccc4)C[C@@]3(O)C2)nc1F ; Oc1ccc([C@H](O)CN2C[C@H]3C[C@H](Oc4ccccc4)C[C@@]3(O)C2)nc1F ; Oc1ccc([C@@H](O)CN2C[C@@H]3C[C@@H](Oc4ccccc4)C[C@]3(O)C2)nc1F ; Oc1ccc([C@H](O)CN2C[C@@H]3C[C@@H](Oc4ccccc4)C[C@]3(O)C2)nc1F]

Using the same methods as Examples 6A/6B/6C/6D, starting from Intermediate 1 and Intermediate 15, provided a mixture of Examples 9A/9B/9C/9D. The mixture was separated using the following chiral HPLC method:

Column: Chiralpak IA (20 mm×250 mm, 5 micron), Flow: 16 mL/min

Mobile phase: Hexane (A), IPA:MeOH 1:1 (B), Isocratic 75:25 (A:B)

Example 9A (chiral HPLC Rt 5.52 min): 12 mg.

LCMS: Rt 0.11 min; MS m/z 375.5 [M+H]⁺; Method D.

¹H NMR (400 MHz, Methanol-d₄) δ 7.31-7.21 (m, 4H), 6.90-6.84 (m, 3H), 4.80-4.76 (m, 1H), 4.67-4.64 (m, 1H), 2.92 (t, J=8.0 Hz, 1H), 2.81 (d, J=9.2 Hz, 1H), 2.75-2.72 (m, 2H), 2.59 (d, J=10.0 Hz, 1H), 2.51-2.44 (m, 2H), 2.22 (dd, J=13.2, 4.8 Hz, 1H), 2.19-2.12 (m, 1H), 2.03 (dd, J=13.2, 5.6 Hz, 1H), 1.84-1.80 (m, 1H).

Example 9B (chiral HPLC Rt 11.83 min): 9 mg.

LCMS: Rt 0.12 min; MS m/z 375.4 [M+H]⁺; Method D.

¹H NMR (400 MHz, Methanol-d₄) δ 7.32-7.21 (m, 4H), 6.90-6.84 (m, 3H), 4.81-4.76 (m, 1H), 4.66 (t, J=6.4 Hz, 1H), 2.88 (t, J=8.0 Hz, 1H), 2.82 (d, J=9.2 Hz, 1H), 2.75 (d, J=6.4 Hz, 2H), 2.67 (d, J=10.0 Hz, 1H), 2.53-2.43 (m, 2H), 2.26 (dd, J=13.2, 5.2 Hz, 1H), 2.18-2.11 (m, 1H), 2.03 (dd, J=13.2, 6.4 Hz, 1H), 1.83-1.78 (m, 1H).

Example 9C (chiral HPLC Rt 12.69 min): 10 mg.

LCMS: Rt 0.12 min; MS m/z 375.4 [M+H]⁺; Method D.

¹H NMR (400 MHz, Methanol-d₄) δ 7.30-7.20 (m, 4H), 6.90-6.84 (m, 3H), 4.81-4.76 (m, 1H), 4.65 (t, J=5.6 Hz, 1H), 2.88-2.84 (m, 1H), 2.80 (d, J=9.2 Hz, 1H), 2.74-2.72 (m, 2H), 2.64 (d, J=9.2 Hz, 1H), 2.51-2.44 (m, 2H), 2.26 (dd, J=13.2, 5.2 Hz, 1H), 2.18-2.11 (m, 1H), 2.00 (dd, J=13.6, 6.0 Hz, 1H), 1.83-1.78 (m, 1H).

Example 9D (chiral HPLC Rt 20.66 min): 10 mg.

LCMS: Rt 0.12 min; MS m/z 375.3 [M+H]⁺; Method D.

¹H NMR (400 MHz, Methanol-d₄) δ 7.32-7.19 (m, 4H), 6.91-6.83 (m, 3H), 4.81-4.75 (m, 1H), 4.67-4.62 (m, 1H), 2.94-2.87 (m, 1H), 2.81 (d, J=9.2 Hz, 1H), 2.76-2.70 (m, 2H), 2.58 (d, J=10.0 Hz, 1H), 2.51-2.41 (m, 2H), 2.26-2.10 (m, 2H), 2.04-1.95 (m, 1H), 1.86-1.79 (m, 1H).

### Examples 10A, 10B, 10C and 10D

(3aS,5S,6aR)-2-((R)-2-(3,5-difluoro-4-hydroxyphenyl)-2-hydroxyethyl)-5-phenoxyhexahydrocyclopenta[c]pyrrol-3a(1H)-ol
(3aS,5S,6aR)-2-((S)-2-(3,5-difluoro-4-hydroxyphenyl)-2-hydroxyethyl)-5-phenoxyhexahydrocyclopenta[c]pyrrol-3a(1H)-ol
(3aR,5R,6aS)-2-((R)-2-(3,5-difluoro-4-hydroxyphenyl)-2-hydroxyethyl)-5-phenoxyhexahydrocyclopenta[c]pyrrol-3a(1H)-ol
(3aR,5R,6aS)-2-((S)-2-(3,5-difluoro-4-hydroxyphenyl)-2-hydroxyethyl)-5-phenoxyhexahydrocyclopenta[c]pyrrol-3a(1H)-ol

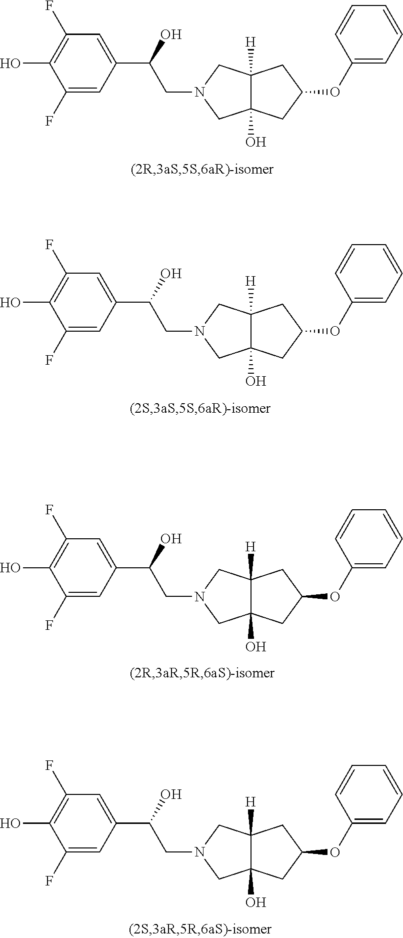 [SMILES: Oc1c(F)cc([C@@H](O)CN2C[C@H]3C[C@H](Oc4ccccc4)C[C@@]3(O)C2)cc1F ; Oc1c(F)cc([C@H](O)CN2C[C@H]3C[C@H](Oc4ccccc4)C[C@@]3(O)C2)cc1F ; Oc1c(F)cc([C@@H](O)CN2C[C@@H]3C[C@@H](Oc4ccccc4)C[C@]3(O)C2)cc1F ; Oc1c(F)cc([C@H](O)CN2C[C@@H]3C[C@@H](Oc4ccccc4)C[C@]3(O)C2)cc1F]

Using the same methods as Examples 6A/6B/6C/6D, starting from Intermediate 1 and Intermediate 20, provided a mixture of Examples 10A/10B/10C/10D. The mixture was separated using the following chiral HPLC method:

Column: Chiralpak IA (20 mm×250 mm, 5 micron), Flow: 18 mL/min

Mobile phase: Hexane (A), 0.1% DEA in EtOH (B), Isocratic 65:35 (A:B)

This gave first a mixture of examples 10A and 10B, followed by example 10C, then example 10D.

Example 10C (chiral HPLC Rt 4.84 min): 37 mg.

LCMS: Rt 0.16 min; MS m/z 392.0 [M+H]⁺; Method D.

¹H NMR (400 MHz, Methanol-d₄) δ 7.25-7.21 (m, 2H), 6.94-6.86 (m, 5H), 4.85-4.80 (m, 1H), 4.67-4.64 (m, 1H), 2.86 (t, J=9.6 Hz, 1H), 2.79 (d, J=9.6 Hz, 1H), 2.71-2.64 (m, 2H), 2.56-2.47 (m, 3H), 2.27 (dd, J=13.6, 5.6 Hz, 1H), 2.20-2.11 (m, 1H), 2.02 (dd, J=13.2, 5.2 Hz, 1H), 1.85-1.80 (m, 1H).

Example 10D (chiral HPLC Rt 7.11 min): 46 mg.

LCMS: Rt 0.16 min; MS m/z 391.8 [M+H]⁺; Method D.

¹H NMR (400 MHz, Methanol-d₄) δ 7.28-7.19 (m, 2H), 6.96-6.84 (m, 5H), 4.84-4.78 (m, 1H), 4.66 (dd, J=8.2, 4.8 Hz, 1H), 2.91 (t, J=9.3 Hz, 1H), 2.81 (d, J=9.5 Hz, 1H), 2.67 (dd, J=12.5, 8.2 Hz, 1H), 2.62-2.54 (m, 2H), 2.52-2.43 (m, 2H), 2.29-2.12 (m, 2H), 2.02 (dd, J=13.4, 5.6 Hz, 1H), 1.84 (dt, J=13.1, 5.1 Hz, 1H).

X-ray structure of Example 10D:

The mixture of 10A and 10B was separated using the method below:

Column: Chiralpak IA (20 mm×250 mm, 5 micron), Flow: 18 mL/min

Mobile phase: Hexane (A), 0.1% DEA in EtOH:MeOH, 1:1 (B), Isocratic 85:15 (A:B)

After separation, both 10A and 10B were further purified by the following preparative HPLC method:

Column: Waters X Bridge C18 (150 mm×21.2 mm), 5.0 μm, Flow: 15 mL/min

Mobile phase: 0.02% NH₄OH in water (A), Acetonitrile (B)

Example 10A (chiral HPLC Rt 5.53 min): 21 mg.

LCMS: Rt 0.14 min; MS m/z 392.0 [M+H]⁺; Method D.

¹H NMR (400 MHz, Methanol-d₄) δ 7.25-7.21 (m, 2H), 6.94-6.86 (m, 5H), 4.84-4.79 (m, 1H), 4.67-4.63 (m, 1H), 2.91 (t, J=9.6 Hz, 1H), 2.80 (d, J=9.6 Hz, 1H), 2.69-2.64 (m, 1H), 2.59-2.54 (m, 2H), 2.49-2.44 (m, 2H), 2.26-2.15 (m, 2H), 2.01 (dd, J=12.8, 5.2 Hz, 1H), 1.87-1.81 (m, 1H).

Example 10B (chiral HPLC Rt 6.31 min): 28 mg.

LCMS: Rt 0.15 min; MS m/z 392.0 [M+H]⁺; Method D.

¹H NMR (400 MHz, Methanol-d₄) δ 7.25-7.21 (m, 2H), 6.89-6.86 (m, 3H), 6.81-6.78 (m, 2H), 4.86-4.81 (m, 1H), 4.60-4.57 (m, 1H), 2.82 (t, J=9.6 Hz, 1H), 2.74-2.63 (m, 3H), 2.50-2.42 (m, 3H), 2.27 (dd, J=13.6, 5.2 Hz, 1H), 2.18-2.11 (m, 1H), 2.01 (dd, J=13.2, 5.2 Hz, 1H), 1.85-1.80 (m, 1H).

### Examples 11A and 11B

(3aS,5S,6aR)-2-((S)-2-(6-chloro-5-hydroxypyridin-2-yl)-2-hydroxyethyl)-5-phenoxyhexahydrocyclopenta[c]pyrrol-3a(1H)-ol
(3aS,5S,6aR)-2-((R)-2-(6-chloro-5-hydroxypyridin-2-yl)-2-hydroxyethyl)-5-phenoxyhexahydrocyclopenta[c]pyrrol-3a(1H)-ol

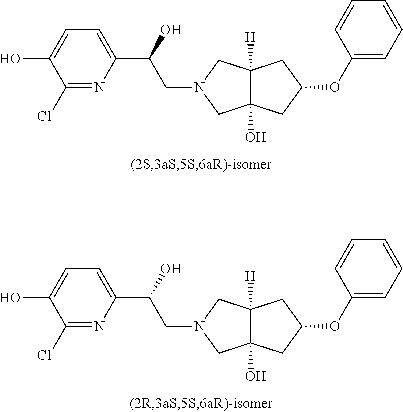 [SMILES: Oc1ccc([C@@H](O)CN2C[C@H]3C[C@H](Oc4ccccc4)C[C@@]3(O)C2)nc1Cl ; Oc1ccc([C@H](O)CN2C[C@H]3C[C@H](Oc4ccccc4)C[C@@]3(O)C2)nc1Cl]

Using the same methods as Examples 6A/6B/6C/6D, starting from Intermediate 2 and Intermediate 19, provided a mixture of Examples 11A and 11B. The mixture was separated using the chiral SFC method below:

Column: Chiralpak IG (10 mm×250 mm, 5 micron), Flow: 12 mL/min

Mobile phase: CO₂ (A), 0.1% DEA in EtOH:MeOH, 1:1 (B), Isocratic 60:40 (A:B)

Example 11A (chiral HPLC Rt 5.80 min): 30 mg.

LCMS: Rt 1.53 min; MS m/z 391.0 [M+H]⁺; Method E.

¹H NMR (400 MHz, Methanol-d₄) δ 7.30-7.18 (m, 4H), 6.90-6.85 (m, 3H), 4.81-4.77 (m, 1H), 4.70-4.67 (m, 1H), 2.88-2.80 (m, 2H), 2.75-2.64 (m, 3H), 2.50-2.44 (m, 2H), 2.29-2.24 (m, 1H), 2.18-2.11 (m, 1H), 2.03-1.98 (m, 1H), 1.82-1.78 (m, 1H).

Example 11B (chiral HPLC Rt 9.52 min): 30 mg.

LCMS: Rt 1.31 min; MS m/z 390.9 [M+H]⁺; Method E.

¹H NMR (400 MHz, Methanol-d₄) δ 7.31-7.20 (m, 4H), 6.90-6.84 (m, 3H), 4.81-4.77 (m, 1H), 4.71-4.67 (m, 1H), 2.94-2.90 (m, 1H), 2.83 (d, J=9.2 Hz, 1H), 2.74 (d, J=6.4 Hz, 2H), 2.60 (d, J=9.2 Hz, 1H), 2.51-2.44 (m, 2H), 2.24-2.11 (m, 2H), 2.02-1.97 (m, 1H), 1.86-1.80 (m, 1H).

### Example 12

A mixture of:
(3aS,5S,6aR)-2-((R)-2-(3,5-difluoro-4-hydroxyphenyl)-2-hydroxyethyl)-5-(2,4-difluorophenoxy)hexahydrocyclopenta[c]pyrrol-3a(1H)-ol
(3aS,5S,6aR)-2-((S)-2-(3,5-difluoro-4-hydroxyphenyl)-2-hydroxyethyl)-5-(2,4-difluorophenoxy)hexahydrocyclopenta[c]pyrrol-3a(1H)-ol
(3aR,5R,6aS)-2-((R)-2-(3,5-difluoro-4-hydroxyphenyl)-2-hydroxyethyl)-5-(2,4-difluorophenoxy)hexahydrocyclopenta[c]pyrrol-3a(1H)-ol
(3aR,5R,6aS)-2-((S)-2-(3,5-difluoro-4-hydroxyphenyl)-2-hydroxyethyl)-5-(2,4-difluorophenoxy)hexahydrocyclopenta[c]pyrrol-3a(1H)-ol

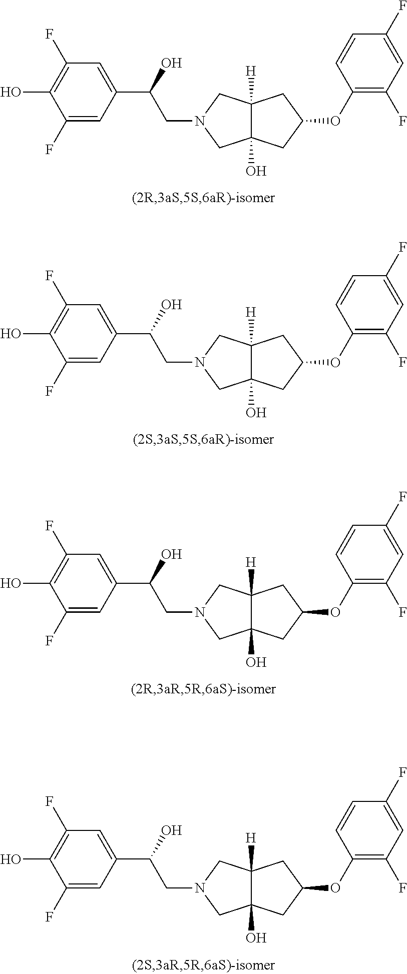 [SMILES: Oc1c(F)cc([C@@H](O)CN2C[C@H]3C[C@H](Oc4ccc(F)cc4F)C[C@@]3(O)C2)cc1F ; Oc1c(F)cc([C@H](O)CN2C[C@H]3C[C@H](Oc4ccc(F)cc4F)C[C@@]3(O)C2)cc1F ; Oc1c(F)cc([C@@H](O)CN2C[C@@H]3C[C@@H](Oc4ccc(F)cc4F)C[C@]3(O)C2)cc1F ; Oc1c(F)cc([C@H](O)CN2C[C@@H]3C[C@@H](Oc4ccc(F)cc4F)C[C@]3(O)C2)cc1F]

### Step 1: A Racemic Mixture of: 1-(3,5-difluoro-4-hydroxyphenyl)-2-((3aS,5S,6aR)-5-(2,4-difluorophenoxy)-3a-hydroxyhexahydrocyclopenta[c]pyrrol-2(1H)-yl)ethan-1-one 1-(3,5-difluoro-4-hydroxyphenyl)-2-((3aR,5R,6aS)-5-(2,4-difluorophenoxy)-3a-hydroxyhexahydrocyclopenta[c]pyrrol-2(1H)-yl)ethan-1-one

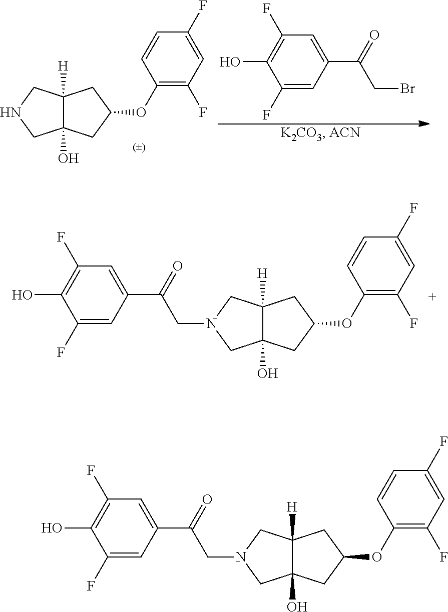

To a solution of Intermediate 10 (74 mg, 0.29 mmol) in Acetonitrile (2 mL) was added K₂CO₃ (120 mg, 0.87 mmol), followed by Intermediate 22 (76 mg, 0.30 mmol), and the reaction was stirred for 4 h at RT. The reaction was filtered and the filtrate was concentrated to provide the title intermediate (123 mg) which was used without further purification.

LCMS: Rt 0.75 min; MS m/z 426.3 [M+H]⁺; Method J.

### Step 2: A Mixture of: (3aS,5S,6aR)-2-((R)-2-(3,5-difluoro-4-hydroxyphenyl)-2-hydroxyethyl)-5-(2,4-difluorophenoxy)hexahydrocyclopenta[c]pyrrol-3a(1H)-ol (3aS,5S,6aR)-2-((S)-2-(3,5-difluoro-4-hydroxyphenyl)-2-hydroxyethyl)-5-(2,4-difluorophenoxy)hexahydrocyclopenta[c]pyrrol-3a(1H)-ol (3aR,5R,6aS)-2-((R)-2-(3,5-difluoro-4-hydroxyphenyl)-2-hydroxyethyl)-5-(2,4-difluorophenoxy)hexahydrocyclopenta[c]pyrrol-3a(1H)-ol (3aR,5R,6aS)-2-((S)-2-(3,5-difluoro-4-hydroxyphenyl)-2-hydroxyethyl)-5-(2,4-difluorophenoxy)hexahydrocyclopenta[c]pyrrol-3a(1H)-ol

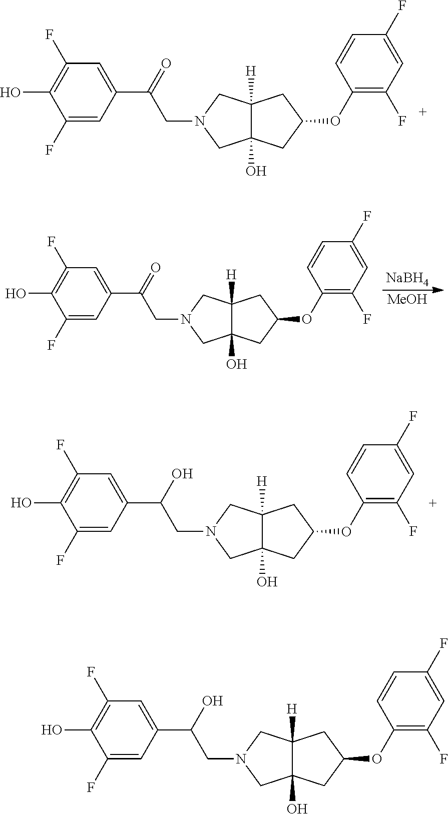

To a solution of 1-(3,5-difluoro-4-hydroxyphenyl)-2-((3aS,5S,6aR)-5-(2,4-difluorophenoxy)-3a-hydroxyhexahydrocyclopenta[c]pyrrol-2(1H)-yl)ethan-1-one and 1-(3,5-difluoro-4-hydroxyphenyl)-2-((3aR,5R,6aS)-5-(2,4-difluorophenoxy)-3a-hydroxyhexahydrocyclopenta[c]pyrrol-2(1H)-yl)ethan-1-one (123 mg, 0.29 mmol) in MeOH (2 mL) was added NaBH₄ (32 mg, 0.87 mmol). The reaction was stirred at RT for 2 h, then concentrated and purified using the following preparative HPLC conditions to provide

Example 12 as a mixture of four diastereomers.

Column: Waters X Bridge C18 (30×50 mm), 5.0 μm

Mobile phase: 10 mM NH₄OH in water (A), Acetonitrile (B); Gradient: 10-30% B.

LCMS: Rt 0.47 min; MS m/z 428.2 [M+H]⁺; Method C.

¹H NMR (400 MHz, Methanol-d₄) δ 7.14-7.05 (m, 1H), 6.99-6.81 (m, 2H), 6.76-6.62 (m, 2H), 4.82-4.76 (m, 1H), 4.58-4.50 (m, 1H), 2.93-2.58 (m, 4H), 2.55-2.38 (m, 3H), 2.31-2.13 (m, 2H), 2.05 (dd, J=13.4, 5.7 Hz, 1H), 1.87-1.74 (m, 1H).

### Example 13

A mixture of:
(3aS,5S,6aR)-5-(2,4-difluorophenoxy)-2-((R)-2-hydroxy-2-(4-hydroxyphenyl)ethyl)hexahydrocyclopenta[c]pyrrol-3a(1H)-ol
(3aS,5S,6aR)-5-(2,4-difluorophenoxy)-2-((S)-2-hydroxy-2-(4-hydroxyphenyl)ethyl)hexahydrocyclopenta[c]pyrrol-3a(1H)-ol
(3aR,5R,6aS)-5-(2,4-difluorophenoxy)-2-((R)-2-hydroxy-2-(4-hydroxyphenyl)ethyl)hexahydrocyclopenta[c]pyrrol-3a(1H)-ol
(3aR,5R,6aS)-5-(2,4-difluorophenoxy)-2-((S)-2-hydroxy-2-(4-hydroxyphenyl)ethyl)hexahydrocyclopenta[c]pyrrol-3a(1H)-ol

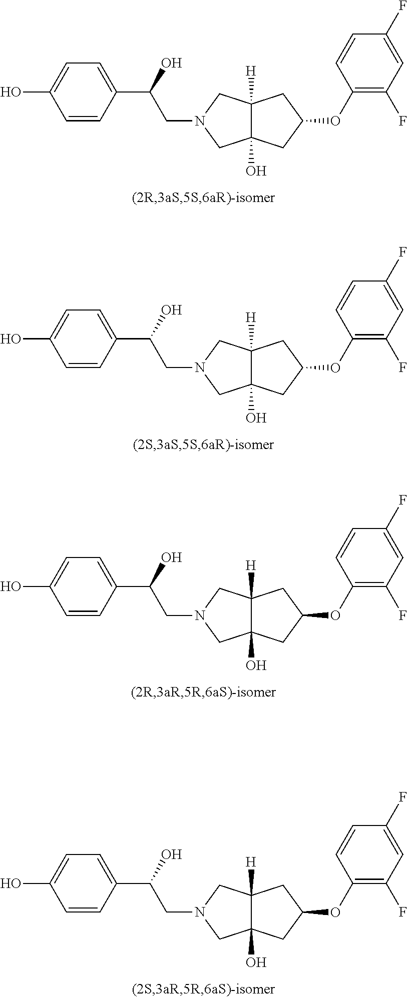 [SMILES: Oc1ccc([C@@H](O)CN2C[C@H]3C[C@H](Oc4ccc(F)cc4F)C[C@@]3(O)C2)cc1 ; Oc1ccc([C@H](O)CN2C[C@H]3C[C@H](Oc4ccc(F)cc4F)C[C@@]3(O)C2)cc1 ; Oc1ccc([C@@H](O)CN2C[C@@H]3C[C@@H](Oc4ccc(F)cc4F)C[C@]3(O)C2)cc1 ; Oc1ccc([C@H](O)CN2C[C@@H]3C[C@@H](Oc4ccc(F)cc4F)C[C@]3(O)C2)cc1]

Using the same method as example 12, starting from Intermediate 10 and Intermediate 21, a mixture of four diastereomers was obtained.

LCMS: Rt 0.63 min; MS m/z 392.1 [M+H]⁺; Method J.

¹H NMR (400 MHz, Methanol-d₄) δ 7.34-7.14 (m, 3H), 7.02-6.94 (m, 1H), 6.92-6.76 (m, 3H), 4.16-3.96 (m, 1H), 3.94-3.32 (m, 4H), 3.15-2.85 (m, 3H), 2.61-2.42 (m, 1H), 2.35-2.17 (m, 2H), 2.14-1.90 (m, 2H).

### Examples 14A and 14B

(3aS,5S,6aR)-2-(3,5-difluoro-4-hydroxyphenethyl)-5-(2-fluorophenoxy)hexahydrocyclopenta[c]pyrrol-3a(1H)-ol
(3aR,5R,6aS)-2-(3,5-difluoro-4-hydroxyphenethyl)-5-(2-fluorophenoxy)hexahydrocyclopenta[c]pyrrol-3a(1H)-ol

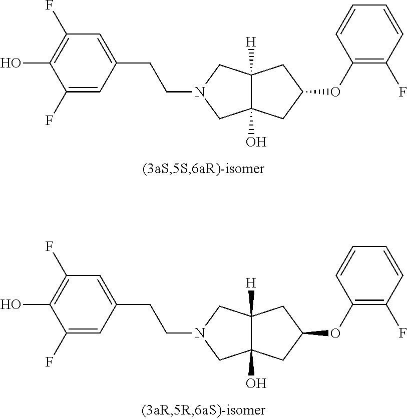 [SMILES: Oc1c(F)cc(CCN2C[C@H]3C[C@H](Oc4ccccc4F)C[C@@]3(O)C2)cc1F ; Oc1c(F)cc(CCN2C[C@@H]3C[C@@H](Oc4ccccc4F)C[C@]3(O)C2)cc1F]

### Step 1: A Racemic Mixture of: (3aS,5S,6aR)-2-(4-(benzyloxy)-3,5-difluorophenethyl)-5-(2-fluorophenoxy)hexahydrocyclopenta[c]pyrrol-3a(1H)-ol (3aR,5R,6aS)-2-(4-(benzyloxy)-3,5-difluorophenethyl)-5-(2-fluorophenoxy)hexahydrocyclopenta[c]pyrrol-3a(1H)-ol

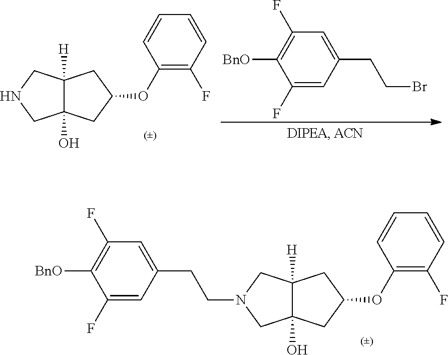

To a solution of Intermediate 3 (100 mg, 0.42 mmol) and Intermediate 23 (151 mg, 0.46 mmol) in acetonitrile (10 mL) was added DIPEA (162 mg, 1.26 mmol) and the reaction was stirred for 5 h at 50° C. The reaction was concentrated and purified by FCC (3% MeOH:DCM) to provide the title intermediate (110 mg).

LCMS: Rt 1.37 min; MS m/z 484.2 [M+H]⁺; Method D.

### Step 2: A Racemic Mixture of: (3aS,5S,6aR)-2-(3,5-difluoro-4-hydroxyphenethyl)-5-(2-fluorophenoxy)hexahydrocyclopenta[c]pyrrol-3a(1H)-ol (3aR,5R,6aS)-2-(3,5-difluoro-4-hydroxyphenethyl)-5-(2-fluorophenoxy)hexahydrocyclopenta[c]pyrrol-3a(1H-ol

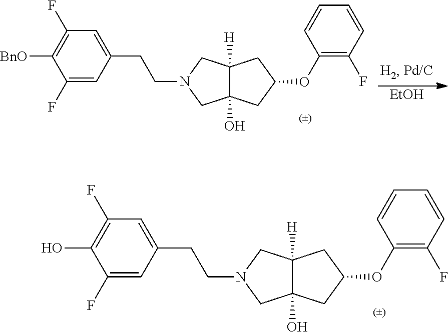 [SMILES: "EtOH" ; O[C@@]12C[C@@H](Oc3ccccc3F)C[C@@H]1CN(CCc1cc(F)c(OCc3ccccc3)c(F)c1)C2 ; Oc1c(F)cc(CCN2C[C@H]3C[C@H](Oc4ccccc4F)C[C@@]3(O)C2)cc1F]

Using the same method as step 3 of Examples 2A/2B, starting with a racemic mixture of (3aS,5S,6aR)-2-(4-(benzyloxy)-3,5-difluorophenethyl)-5-(2-fluorophenoxy)hexahydrocyclopenta[c]pyrrol-3a(1H)-ol and (3aR,5R,6aS)-2-(4-(benzyloxy)-3,5-difluorophenethyl)-5-(2-fluorophenoxy)hexahydrocyclopenta[c]pyrrol-3a(1H)-ol (110 mg), provided a racemic mixture of Examples 14A and 14B (50 mg).

LCMS: Rt 0.23 min; MS m/z 394.2 [M+H]⁺; Method D.

### Step 3: Chiral Separation of: (3aS,5S,6aR)-2-(3,5-difluoro-4-hydroxyphenethyl)-5-(2-fluorophenoxy)hexahydrocyclopenta[c]pyrrol-3a(1H)-ol (3aR,5R,6aS)-2-(3,5-difluoro-4-hydroxyphenethyl)-5-(2-fluorophenoxy)hexahydrocyclopenta[c]pyrrol-3a(1H)-ol

The two enantiomers were separated using the chiral HPLC method below:

Column: Chiralpak IA (20 mm×250 mm, 5 micron), Flow: 20 mL/min

Mobile phase: Hexane (A), MeOH:IPA 1:1 (B), Isocratic 90:10 (A:B)

Example 14A (chiral HPLC Rt 6.46 min): 15 mg.

LCMS: Rt 0.22 min; MS m/z 394.0 [M+H]⁺; Method D.

¹H NMR (400 MHz, Methanol-d₄) δ 7.09-7.00 (m, 3H), 6.93-6.88 (m, 1H), 6.82-6.76 (m, 2H), 4.87-4.82 (m, 1H), 2.83-2.76 (m, 2H), 2.72-2.61 (m, 4H), 2.54-2.47 (m, 3H), 2.27-2.18 (m, 2H), 2.08 (dd, J=13.6, 6.0 Hz, 1H), 1.84-1.78 (m, 1H).

Example 14B (chiral HPLC Rt 5.90 min): 12 mg.

LCMS: Rt 0.21 min; MS m/z 394.0 [M+H]⁺; Method D.

¹H NMR (400 MHz, Methanol-d₄) δ 7.09-7.00 (m, 3H), 6.93-6.88 (m, 1H), 6.85-6.75 (m, 2H), 4.87-4.81 (m, 1H), 2.83-2.75 (m, 2H), 2.72-2.60 (m, 4H), 2.53-2.47 (m, 3H), 2.27-2.16 (m, 2H), 2.08 (dd, J=13.2, 5.6 Hz, 1H), 1.84-1.78 (m, 1H).

### Examples 15A and 15B

(3aS,5S,6aR)-2-(3,5-difluoro-4-hydroxyphenethyl)-5-(2,4-difluorophenoxy)hexahydrocyclopenta[c]pyrrol-3a(1H)-ol
(3aR,5R,6aS)-2-(3,5-difluoro-4-hydroxyphenethyl)-5-(2,4-difluorophenoxy)hexahydrocyclopenta[c]pyrrol-3a(1H)-ol

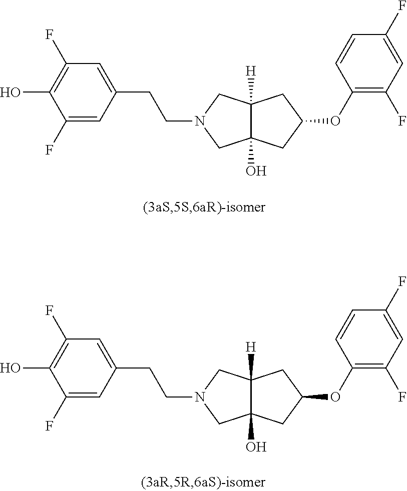 [SMILES: Oc1c(F)cc(CCN2C[C@H]3C[C@H](Oc4ccc(F)cc4F)C[C@@]3(O)C2)cc1F ; Oc1c(F)cc(CCN2C[C@@H]3C[C@@H](Oc4ccc(F)cc4F)C[C@]3(O)C2)cc1F]

Using the same method as examples 14A/14B, starting from Intermediate 10 and Intermediate 23, provided a racemic mixture of 15A and 15B. The material was first purified using the preparative HPLC method below:

Column: Kinetex (150 mm×21.2 mm), 5.0 μm, Flow: 20 mL/min

Mobile phase: 10 mM ammonium acetate in water (A), Acetonitrile (B)

Gradient Program:

| Time (min) | Mobile phase (% B) |
|---|---|
| 0 | 10 |
| 2 | 20 |
| 7 | 40 |

The solvent was removed, water was added and the solution was extracted with 10% MeOH in CHCl₃ (3×10 mL), dried with Na₂SO₄, and concentrated to give a mixture of 15A and 15B (30 mg). The mixture was separated using the chiral HPLC method below:

Column: Chiralpak IA (20 mm×250 mm, 5 micron), Flow: 18 mL/min

Mobile phase: Hexane (A), MeOH:EtOH 1:1 (B), Isocratic 85:15 (A:B)

Example 15A (chiral HPLC Rt 5.26 min): 10 mg.

LCMS: Rt 0.26 min; MS m/z 412.0 [M+H]⁺; Method D.

¹H NMR (400 MHz, Methanol-d₄) δ 7.06-6.99 (m, 1H), 6.98-6.90 (m, 1H), 6.89-6.74 (m, 3H), 4.80-4.73 (m, 1H), 2.81 (d, J=9.2 Hz, 1H), 2.78-2.56 (m, 5H), 2.54-2.45 (m, 3H), 2.25-2.14 (m, 2H), 2.07 (dd, J=13.6, 5.6 Hz, 1H), 1.83-1.74 (m, 1H).

Example 15B (chiral HPLC Rt 6.22 min): 10 mg.

LCMS: Rt 0.25 min; MS m/z 412.1 [M+H]⁺; Method D.

¹H NMR (400 MHz, Methanol-d₄) δ 7.06-6.98 (m, 1H), 6.98-6.90 (m, 1H), 6.89-6.74 (m, 3H), 4.80-4.73 (m, 1H), 2.81 (d, J=9.2 Hz, 1H), 2.78-2.56 (m, 5H), 2.54-2.45 (m, 3H), 2.25-2.14 (m, 2H), 2.07 (dd, J=13.6, 5.6 Hz, 1H), 1.83-1.74 (m, 1H).

### Examples 16A and 16B

(3aS,5S,6aR)-2-(3,5-difluoro-4-hydroxyphenethyl)-5-phenoxyhexahydrocyclopenta[c]pyrrol-3a(1H)-ol
(3aR,5R,6aS)-2-(3,5-difluoro-4-hydroxyphenethyl)-5-phenoxyhexahydrocyclopenta[c]pyrrol-3a(1H)-ol

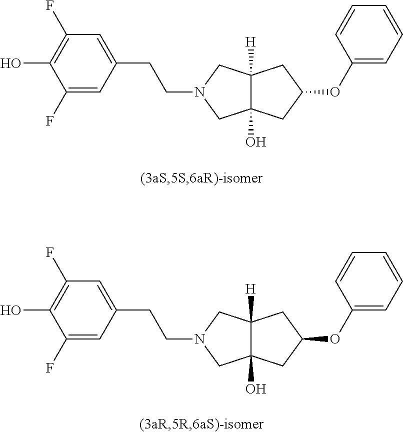 [SMILES: Oc1c(F)cc(CCN2C[C@H]3C[C@H](Oc4ccccc4)C[C@@]3(O)C2)cc1F ; Oc1c(F)cc(CCN2C[C@@H]3C[C@@H](Oc4ccccc4)C[C@]3(O)C2)cc1F]

Using the same method as examples 14A/14B, starting from Intermediate 1 and Intermediate 23, provided a racemic mixture of 16A and 16B. The mixture was separated using the chiral HPLC method below:

Column: Chiralpak IA (20 mm×250 mm, 5 micron), Flow: 15 mL/min

Mobile phase: Hexane (A), 0.1% TFA in IPA:MeOH 4:6 (B), Isocratic 90:10 (A:B)

Example 16A (chiral HPLC Rt 7.15 min): 26 mg.

LCMS: Rt 0.17 min; MS m/z 375.8 [M+H]⁺; Method D.

¹H NMR (400 MHz, Methanol-d₄) δ 7.25-7.21 (m, 2H), 6.90-6.85 (m, 3H), 6.79 (d, J=8.8 Hz, 2H), 4.85-4.80 (m, 1H), 2.83-2.79 (m, 2H), 2.71-2.62 (m, 4H), 2.55-2.49 (m, 3H), 2.27-2.18 (m, 2H), 2.04 (dd, J=12.8, 5.2 Hz, 1H), 1.86-1.80 (m, 1H).

Example 16B (chiral HPLC Rt 8.58 min): 27 mg.

LCMS: Rt 0.12 min; MS m/z 375.9 [M+H]⁺; Method D.

¹H NMR (400 MHz, Methanol-d₄) δ 7.25-7.21 (m, 2H), 6.90-6.85 (m, 3H), 6.79 (d, J=9.2 Hz, 2H), 4.85-4.80 (m, 1H), 2.83-2.78 (m, 2H), 2.71-2.62 (m, 4H), 2.55-2.49 (m, 3H), 2.27-2.16 (m, 2H), 2.04 (dd, J=12.8, 5.2 Hz, 1H), 1.86-1.80 (m, 1H).

### Examples 17A and 17B

(3aS,5S,6aR)-5-((3-fluoropyridin-2-yl)oxy)-2-(4-hydroxyphenethyl)hexahydrocyclopenta[c]pyrrol-3a(1H)-ol
(3aR,5R,6aS)-5-((3-fluoropyridin-2-yl)oxy)-2-(4-hydroxyphenethyl)hexahydrocyclopenta[c]pyrrol-3a(1H)-ol

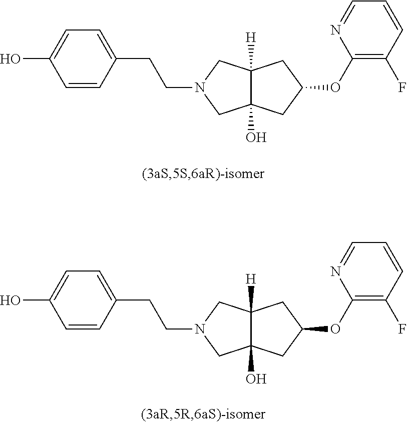 [SMILES: Oc1ccc(CCN2C[C@H]3C[C@H](Oc4ncccc4F)C[C@@]3(O)C2)cc1 ; Oc1ccc(CCN2C[C@@H]3C[C@@H](Oc4ncccc4F)C[C@]3(O)C2)cc1]

Using the same method as examples 14A/14B, starting from Intermediate 11 and Intermediate 24, provided a racemic mixture of 17A and 17B. The mixture was separated using the chiral HPLC method below:

Column: Chiralpak IG (4.6 mm×250 mm, 5 micron), Flow: 3 mL/min

Mobile phase: Hexane (A), EtOH (B), Isocratic 85:15 (A:B)

Example 17A (chiral HPLC Rt 11.07 min): 5 mg.

LCMS: Rt 0.12 min; MS m/z 359.4 [M+H]⁺; Method D.

¹H NMR (400 MHz, Methanol-d₄) δ 7.90 (dd, J=4.8, 1.2 Hz, 1H), 7.46-7.41 (m, 1H), 7.04 (d, J=8.4 Hz, 2H), 6.93-6.89 (m, 1H), 6.69 (d, J=8.8 Hz, 2H), 5.51-5.47 (m, 1H), 2.94 (t, J=8.4 Hz, 1H), 2.75-2.68 (m, 4H), 2.67-2.60 (m, 2H), 2.57-2.50 (m, 1H), 2.44-2.35 (m, 2H), 2.24-2.17 (m, 1H), 2.08-2.03 (m, 1H), 1.99-1.94 (m, 1H).

Example 17B (chiral HPLC Rt 21.48 min): 5 mg.

LCMS: Rt 0.12 min; MS m/z 359.4 [M+H]⁺; Method D.

¹H NMR (400 MHz, Methanol-d₄) δ 7.90-7.89 (m, 1H), 7.46-7.41 (m, 1H), 7.04 (d, J=8.4 Hz, 2H), 6.93-6.89 (m, 1H), 6.69 (d, J=8.4 Hz, 2H), 5.51-5.47 (m, 1H), 2.97 (t, J=8.8 Hz, 1H), 2.76-2.62 (m, 6H), 2.59-2.52 (m, 1H), 2.46-2.35 (m, 2H), 2.25-2.17 (m, 1H), 2.06 (dd, J=12.8, 6.0 Hz, 1H), 2.00-1.94 (m, 1H).

### Examples 18A and 18B

(3aS,5S,6aR)-5-((2-fluoropyridin-3-yl)oxy)-2-(4-hydroxyphenethyl)hexahydrocyclopenta[c]pyrrol-3a(1H)-ol
(3aR,5R,6aS)-5-((2-fluoropyridin-3-yl)oxy)-2-(4-hydroxyphenethyl)hexahydrocyclopenta[c]pyrrol-3a(1H)-ol

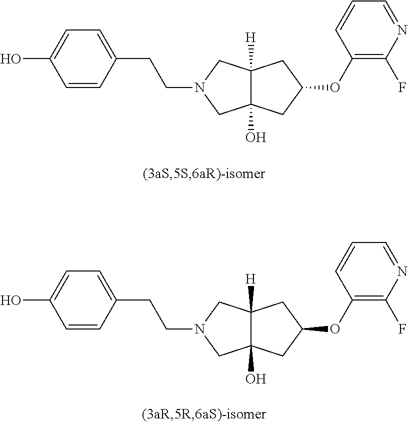 [SMILES: Oc1ccc(CCN2C[C@H]3C[C@H](Oc4cccnc4F)C[C@@]3(O)C2)cc1 ; Oc1ccc(CCN2C[C@@H]3C[C@@H](Oc4cccnc4F)C[C@]3(O)C2)cc1]

Using the same method as examples 14A/14B, starting from Intermediate 12 and Intermediate 24, provided a racemic mixture of 18A and 18B. The mixture was separated using the chiral HPLC method below:

Column: Chiralpak IA (20 mm×250 mm, 5 micron), Flow: 18 mL/min

Mobile phase: Hexane (A), IPA:MeOH 1:1 (B), Isocratic 85:15 (A:B)

Example 18A (chiral HPLC Rt 5.13 min): 12 mg.

LCMS: Rt 0.11 min; MS m/z 359.3 [M+H]⁺; Method D.

¹H NMR (400 MHz, Methanol-d₄) δ 7.67 (d, J=4.8 Hz, 1H), 7.46 (t, J=8.8 Hz, 1H), 7.24-7.20 (m, 1H), 7.03 (d, J=8.4 Hz, 2H), 6.68 (d, J=8.4 Hz, 2H), 2.86 (d, J=9.2 Hz, 1H), 2.76-2.68 (m, 3H), 2.66-2.59 (m, 2H), 2.56-2.46 (m, 3H), 2.29-2.19 (m, 2H), 2.08 (dd, J=13.6, 5.6 Hz, 1H), 1.90-1.84 (m, 1H). 1H under solvent peak.

Example 18B (chiral HPLC Rt 6.20 min): 15 mg.

LCMS: Rt 0.11 min; MS m/z 359.5 [M+H]⁺; Method D.

¹H NMR (400 MHz, Methanol-d₄) δ 7.69 (d, J=4.8 Hz, 1H), 7.48 (t, J=9.6 Hz, 1H), 7.26-7.23 (m, 1H), 7.05 (d, J=8.4 Hz, 2H), 6.70 (d, J=8.4 Hz, 2H), 2.88 (d, J=9.6 Hz, 1H), 2.78-2.70 (m, 3H), 2.66-2.61 (m, 2H), 2.58-2.49 (m, 3H), 2.32-2.21 (m, 2H), 2.11 (dd, J=13.2, 5.6 Hz, 1H), 1.92-1.87 (m, 1H). 1H under solvent peak.

### Example 19

A racemic mixture of:
(3aS,5S,6aR)-5-(2,4-difluorophenoxy)-2-(2-(5-hydroxypyridin-2-yl)ethyl)hexahydrocyclopenta[c]pyrrol-3a(1H)-ol
(3aR,5R,6aS)-5-(2,4-difluorophenoxy)-2-(2-(5-hydroxypyridin-2-yl)ethyl)hexahydrocyclopenta[c]pyrrol-3a(1H)-ol

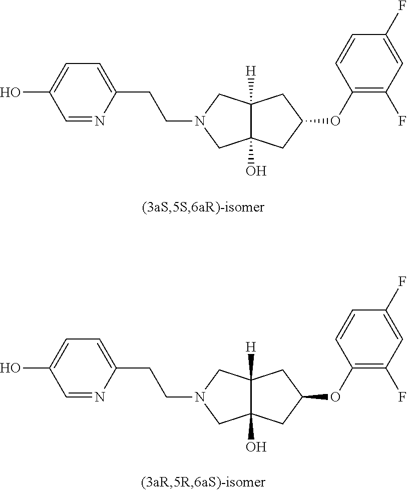 [SMILES: Oc1ccc(CCN2C[C@H]3C[C@H](Oc4ccc(F)cc4F)C[C@@]3(O)C2)nc1 ; Oc1ccc(CCN2C[C@@H]3C[C@@H](Oc4ccc(F)cc4F)C[C@]3(O)C2)nc1]

### Step 1: A Racemic Mixture of: (3aS,5S,6aR)-5-(2,4-difluorophenoxy)-2-(2-(5-((4-methoxybenzyl)oxy)pyridin-2-yl)ethyl)hexahydrocyclopenta[c]pyrrol-3a(1H)-ol (3aR,5R,6aS)-5-(2,4-difluorophenoxy)-2-(2-(5-((4-methoxybenzyl)oxy)pyridin-2-yl)ethyl)hexahydrocyclopenta[c]pyrrol-3a(1H)-ol

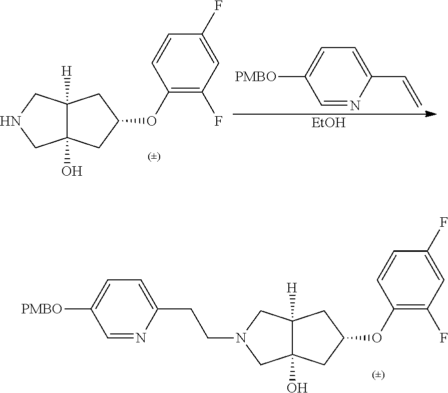

To a solution of Intermediate 13 (71 mg, 0.30 mmol) in EtOH (0.2 mL) was added Intermediate 10 (50 mg, 0.20 mmol). The reaction was stirred for 96 h at 90° C., then concentrated and purified by preparative TLC (10% MeOH:EtOAc) to provide the title intermediate (50 mg) as a yellow oil.

LCMS: Rt 0.76 min; MS m/z 497.2 [M+H]⁺; Method J.

### Step 2: A Racemic Mixture of: (3aS,5S,6aR)-5-(2,4-difluorophenoxy)-2-(2-(5-hydroxypyridin-2-yl)ethyl)hexahydrocyclopenta[c]pyrrol-3a(1H)-ol (3aR,5R,6aS)-5-(2,4-difluorophenoxy)-2-(2-(5-hydroxypyridin-2-yl)ethyl)hexahydrocyclopenta[c]pyrrol-3a(1H-ol

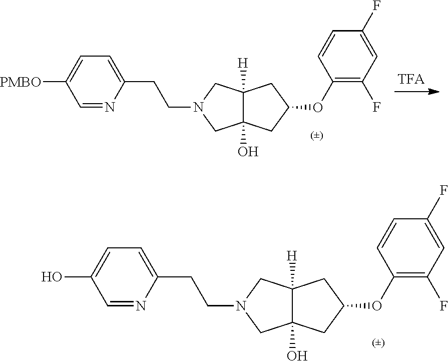

Using the same method as step 2 of Example 1A/1B/1C/1D, starting with a mixture of (3aS,5S,6aR)-5-(2,4-difluorophenoxy)-2-(2-(5-((4-methoxybenzyl)oxy)pyridin-2-yl)ethyl)hexahydrocyclopenta[c]pyrrol-3a(1H)-ol and (3aR,5R,6aS)-5-(2,4-difluorophenoxy)-2-(2-(5-((4-methoxybenzyl)oxy)pyridin-2-yl)ethyl)hexahydrocyclopenta[c]pyrrol-3a(1H)-ol (50 mg), provided the title compounds (8.0 mg).

LCMS: Rt 0.79 min; MS m/z 377.2 [M+H]⁺; Method I.

¹H NMR (400 MHz, Methanol-d₄) δ 8.28 (d, J=2.1 Hz, 1H), 7.99-7.86 (m, 2H), 7.20 (dt, J=9.1, 5.5 Hz, 1H), 7.04-6.95 (m, 1H), 6.94-6.84 (m, 1H), 5.02-4.95 (m, 3H), 3.98 (br s, 1H), 3.76-3.44 (m, 5H), 3.04-2.93 (m, 1H), 2.56 (dd, J=14.3, 4.9 Hz, 1H), 2.37-2.27 (m, 1H), 2.19-2.00 (m, 2H).

### Example 20

A racemic mixture of:
(3aS,5S,6aR)-5-((2-fluoropyridin-3-yl)oxy)-2-(2-(5-hydroxypyridin-2-yl)ethyl)hexahydrocyclopenta[c]pyrrol-3a(1H)-ol
(3aR,5R,6aS)-5-((2-fluoropyridin-3-yl)oxy)-2-(2-(5-hydroxypyridin-2-yl)ethyl)hexahydrocyclopenta[c]pyrrol-3a(1H-ol

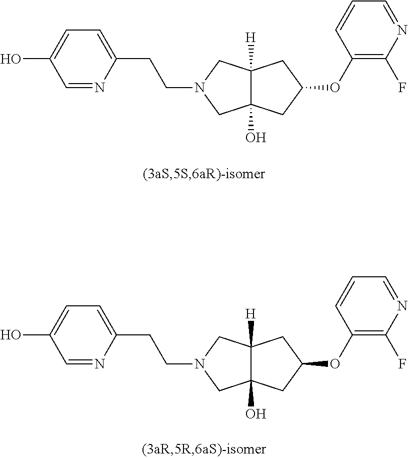 [SMILES: Oc1ccc(CCN2C[C@H]3C[C@H](Oc4cccnc4F)C[C@@]3(O)C2)nc1 ; Oc1ccc(CCN2C[C@@H]3C[C@@H](Oc4cccnc4F)C[C@]3(O)C2)nc1]

Using the same method as example 19, starting from Intermediate 12 and Intermediate 13, provided the title compounds (12 mg).

LCMS: Rt 0.10 min; MS m/z 360.4 [M+H]⁺; Method D.

¹H NMR (400 MHz, Methanol-d₄) δ 7.96 (d, J=2.4 Hz, 1H), 7.67-7.65 (m, 1H), 7.45-7.40 (m, 1H), 7.22-7.12 (m, 3H), 4.86-4.81 (m, 1H), 2.89-2.84 (m, 3H), 2.76-2.70 (m, 3H), 2.56 (dd, J=9.2, 2.8 Hz, 1H), 2.51-2.44 (m, 2H), 2.25-2.19 (m, 2H), 2.04 (dd, J=13.6, 5.6 Hz, 1H), 1.86-1.81 (m, 1H).

Biological Assays and Data

The activity of a compound according to the present disclosure can be assessed by the following in vitro & in vivo methods.

### Example 1: NR2B Rat Cortical Neuron Calcium Influx Assay Protocol

Embryonic day 18 timed pregnant Sprague Dawley rats were euthanized according to Institutional Animal Care and Use Committee (IACUC) protocol. After cutting medially through the skin and exposing the uterus and embryos, fetuses were removed and placed in cold Hibernate medium. Each embryo's brain was extracted and cerebral cortices were isolated by removing the midbrain and meninges. The dissected cortices were then dissociated into the neurons using papain dissociation system (Worthington Biochemical Corporation) according the manufacturer's protocol.

Dissociated neurons were counted and plated into 384-well poly-D-lysine coated plates (Corning® BioCoat™) at a density of 20,000 cells/well in 30 μL of Neurobasal/B27 complete medium. Neurons were cultured at 37° C. for 2 days. On the assay day, medium was removed and cells were incubated with 20 μL/well of calcium dye (Calcium 6 Assay Kit, Molecular Devices) suspended in HBSS with 1.8 mM Ca²⁺ (Ca-HBSS) according to the manufacturer's instruction.

Compounds of interest from 10 mM stock were serially diluted into 3× of desired concentrations in 1.8 mM Ca-HBSS, and 10 μL were added to the wells. Compound and the neurons were incubated at 37° C. for 2 hours in the dark.

On FDSS7000EX (Hamamatsu Photonics), a fluorescence measuring instrument, 10 μL of 4× ligand solution containing glutamate and glycine made in 1.8 mM Ca²⁺-HBSS were added to each well. The fluorescent signals were collected before and after the addition of ligands for a total of 2 minutes. The data were converted to a ratio of the peak fluorescence to the fluorescence at the beginning of the measurement.

Each data point was measured in duplicates. Dose response curves were used to identify IC₅₀ and maximal inhibition values. IC₅₀ represents the concentration in μM of compound at which there is a half-maximal compound effect. Maximal inhibition of a compound is expressed as a percent of the highest inhibition of activity over a no compound control.

TABLE 1
NR2B rat cortical neuron calcium influx assay data.

| Example | IC₅₀ (μM) |
|---|---|
| 1A | 0.074 |
| 1B | 0.00074 |
| 1C | 0.0011 |
| 1D | 0.011 |
| 2A | 0.011 |
| 2B | 0.0022 |
| 2C | 0.77 |
| 2D | 0.95 |
| 3A | >10 |
| 3B | >10 |
| 3C | 0.14 |
| 3D | 0.024 |
| 4A | >10 |
| 4B | >10 |
| 4C | 0.031 |
| 4D | 0.035 |
| 5A | 0.24 |
| 5B | 0.028 |
| 5C | 0.9 |
| 5D | 3.7 |
| 6A | 1.5 |
| 6B | 0.083 |
| 6C | 0.0055 |
| 6D | 0.94 |
| 7A | 0.04 |
| 7B | 1.6 |
| 7C | 0.0024 |
| 7D | 0.0032 |
| 8A | 0.14 |
| 8B | 3.6 |
| 8C | 0.0081 |
| 8D | 0.0028 |
| 9A | 0.59 |
| 9B | >10 |
| 9C | 0.013 |
| 9D | 0.0089 |
| 10A | 0.15 |
| 10B | 1.7 |
| 10C | 0.014 |
| 10D | 0.006 |
| 11A | 0.0035 |
| 11B | 0.014 |
| 12 | 0.15 |
| 13 | 0.041 |
| 14A | 0.009 |
| 14B | 0.21 |
| 15A | 0.019 |
| 15B | 1.4 |
| 16A | 0.041 |
| 16B | 0.53 |
| 17A | 0.053 |
| 17B | 1.2 |
| 18A | 0.57 |
| 18B | 4.0 |
| 19 | 0.018 |
| 20 | 0.82 |

### Example 2. Microsome and Hepatocyte Assay Protocols

Microsome Incubations: The experiments were performed in 96-well format with shaking incubation at 37° C. on an automated platform. Test compounds, at a concentration of 10 mM in DMSO, were diluted 1:5000 into a 100 mM potassium phosphate, pH 7.4 (KPi) solution containing cofactor (2 mM NADPH, 4 mM MgCl₂) to a concentration of 2 μM. The reaction was initiated by adding equal volume to rat or human liver microsomal protein (1 mg/mL) suspended in 100 mM KPi buffer. At specific reaction time points (0, 5, 15, and 30 minutes), reaction aliquots were removed and reactions were terminated by the addition of three volumes of acetonitrile containing the analytical internal standard (0.4 μM glyburide). The samples were then centrifuged at 4000×g at 4° C. for 10 minutes, and the supernatants were analyzed by LC/MS/MS for quantification of the remaining test compound. The percentage of test compound remaining, relative to time zero minute incubation, was used to estimate the in vitro elimination-rate constant (k_{mic}), which was subsequently used to calculate the in vitro metabolic clearance rates.

Hepatocyte Incubations: The experiments were performed in 96-well format with shaking incubation at 37° C. on an automated platform. Test compounds, at a concentration of 10 mM in DMSO, were diluted 1:5000 into a Leibovitz's L15 medium (L-15) solution to a concentration of 2 μM. The reaction was initiated by adding equal volume to suspended rat or human hepatocytes at 2 million cells/mL in L-15 media solution. At specific reaction time points (0, 10, 20, 40, 60, and 80 minutes), reaction aliquots were removed and reactions were terminated by the addition of three volumes of acetonitrile containing the analytical internal standard (0.4 μM glyburide). The samples were then centrifuged at 4000×g at 4° C. for 10 minutes, and the supernatants were analyzed by LC/MS/MS for quantification of the remaining test compound. The percentage of test compound remaining, relative to time zero minute incubation, was used to estimate the in vitro elimination-rate constant (k_{mic}), which was subsequently used to calculate the in vitro metabolic clearance rates.

LC/MS/MS Analysis: Samples were analyzed on a high performance liquid chromatography (HPLC)-tandem mass spectrometry (LC/MS/MS) system consisting of Shimadzu 30 series autosampler and HPLC pump coupled to an AB Sciex API6500. Compound specific parameters (precursor ion, product ion, declustering potential, and collision energy for single reaction monitoring) were obtained by automatic tuning using the Multiquant software V3.0. Samples were loaded onto an ACE 3 C18, 2.1 mm×30 mm, 3 μm column by means of the Shimadzu 30 series autosampler. The components were eluted with a gradient of 0.1% formic acid in water (mobile phase A) and 0.1% formic acid in acetonitrile (mobile phase B) at a flow of 700 μL/min using the following gradient: 0 min 2% B; 0.25 min 2% B; 1.00 min 98% B; 1.55 min 98% B; 1.95 min 2% B; 2.00 min 2% B. The analyte concentration was calculated from the chromatographic peak area ratio of analyte to internal standard (glyburide, m/z 494→169), using Multiquant software V3.0 (Sciex, Framingham, MA).

### Example 3. hERG Qpatch Assay Protocol

This assay was performed by the method described in Skepper et al., J. Med. Chem. 2020, 63, 7773-7816:

hERG expressing cell lines were produced in-house at Novartis using CHO-K1 T-Rex inducible plasmid system (Invitrogen) as described previously (Cao et al., Assay Drug Dev. Technol. 2010, 8, 766-780). Cell lines were maintained in Ham's F12 nutrient mixture containing 10% FBS, blasticidin (10 mg/mL; InvivoGen), hygromycin B (200 mg/mL; InvivoGen), Zeocin (200 mg/mL, Invitrogen), and neomycin (200 mg/mL, Invitrogen) using SelecT automated cell culture system (TAP Biosystems, Cambridge, U.K.). hERG and hCav1.2 channels expression was induced with tetracycline (0.25-1 μg/mL, Invitrogen) at least 24 h prior to the experiment.

hERG currents were recorded using the Qpatch automated patch clamp systems (Sophion Bioscience Inc., North Brunswick, NJ) in the whole (single) cell configuration. hERG expressing CHO-K1 cells were harvested with Detachin (Genlantis) and stored in the modified serum-free SFM-2 media (Life Technologies) at room temperature. The extracellular solution contained (in mM) NaCl (145), KCl (4), MgCl₂ (1), CaCl₂ (2), and HEPES (10), pH 7.4, with NaOH. The intracellular solution contained KCl (135), MgCl₂ (1.75), CaCl₂ (5.4), EGTA (10), K2-ATP (4), and HEPES (10), pH 7.2, with KOH. After whole cell configuration was achieved, the cell was held at −90 mV, and a 0.1 s pulse to −50 mV was delivered to measure the leaking current, which was subtracted from the tail current online. Then the cell was depolarized to +20 mV for 4 s (prepulse), followed by a 4 s test pulse to −50 mV to reveal the hERG tail current. To monitor changes in the current amplitude, this voltage protocol was repeatedly applied every 20 s. Test compounds were first diluted in DMSO for six dose-response experiments and then dissolved in the extracellular solution using Freedom EVO liquid handling robotic system (Tecan, Männedorf, Switzerland). The final DMSO concentration in samples was 0.3% v/v. Amitriptyline (Sigma) was tested as a positive control. Data were analyzed using in-house developed MatLab-based program (MathWorks, Natick, MA).

TABLE 2
Comparison of in vitro ADME and hERG Qpatch data between matched pairs
containing the hydroxy core (present diclosure) vs. des-hydroxy cores
(comparative compounds).

| | |
|---|---|
| Structure | Example |

| | | | | |
|---|---|---|---|---|
| Rat | Human | Rat | Human | |
| Microsome | Microsome | Hepatocyte | Hepatocyte | hERG Qpatch |
| CLᵢₙₜ | CLᵢₙₜ | CLᵢₙₜ | CLᵢₙₜ | IC₅₀ (μM) |

| | |
|---|---|
| 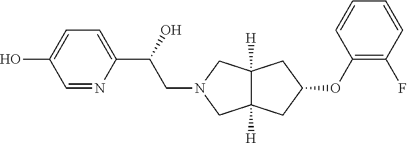 [SMILES: Oc1ccc([C@H](O)CN2C[C@H]3C[C@H](Oc4ccccc4F)C[C@H]3C2)nc1] | Example 11 from WO 2016/049165 |

| | | | | |
|---|---|---|---|---|
| 229 | 141 | 188 | 56 | 2.4 |

| | |
|---|---|
| 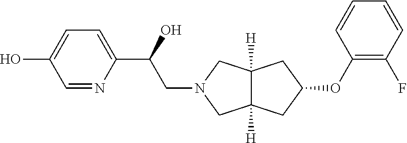 [SMILES: Oc1ccc([C@@H](O)CN2C[C@H]3C[C@H](Oc4ccccc4F)C[C@H]3C2)nc1] | Example 10 from WO 2016/049165 |

| | | | | |
|---|---|---|---|---|
| 248 | 152 | 155 | 49 | 2.3 |

| | |
|---|---|
| 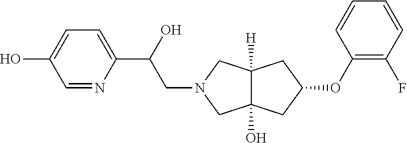 [SMILES: Oc1ccc(C(O)CN2C[C@H]3C[C@H](Oc4ccccc4F)C[C@@]3(O)C2)nc1] | Example 1B |

| | | | | |
|---|---|---|---|---|
| 241 | 41 | 80 | 7 | 14 |

| | |
|---|---|
| 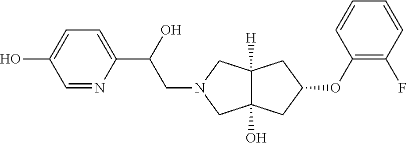 [SMILES: Oc1ccc(C(O)CN2C[C@H]3C[C@H](Oc4ccccc4F)C[C@@]3(O)C2)nc1] | Example 1C |

| | | | | |
|---|---|---|---|---|
| 155 | 37 | 157 | 11 | 22 |

| | |
|---|---|
| 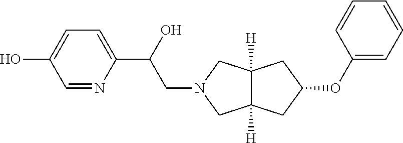 [SMILES: Oc1ccc(C(O)CN2C[C@H]3C[C@H](Oc4ccccc4)C[C@H]3C2)nc1] | Table 3 from WO 2016/049165 (isomer 1) |

| | | | | |
|---|---|---|---|---|
| 266 | 106 | 134 | 30 | 2.0 |

| | |
|---|---|
| 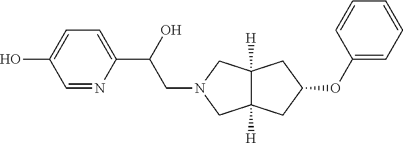 [SMILES: Oc1ccc(C(O)CN2C[C@H]3C[C@H](Oc4ccccc4)C[C@H]3C2)nc1] | Table 3 of WO 2016/049165 (isomer 2) |

| | | | | |
|---|---|---|---|---|
| >700 | 179 | 82 | 93 | 2.7 |

| | |
|---|---|
| 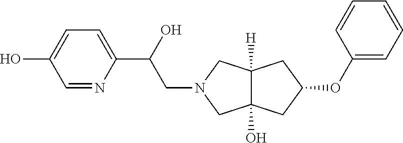 [SMILES: Oc1ccc(C(O)CN2C[C@H]3C[C@H](Oc4ccccc4)C[C@@]3(O)C2)nc1] | Example 7C |

| | | | | |
|---|---|---|---|---|
| 66 | <25 | 155 | 11 | 28 |

| | |
|---|---|
| 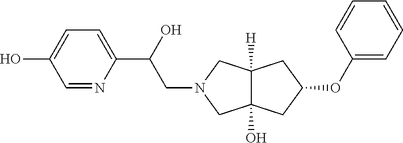 [SMILES: Oc1ccc(C(O)CN2C[C@H]3C[C@H](Oc4ccccc4)C[C@@]3(O)C2)nc1] | Example 7D |

| | | | | |
|---|---|---|---|---|
| 75 | 44 | 163 | ND | 19 |

| | |
|---|---|
| 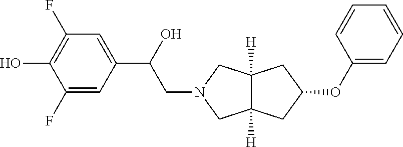 [SMILES: Oc1c(F)cc(C(O)CN2C[C@H]3C[C@H](Oc4ccccc4)C[C@H]3C2)cc1F] | Mixture of two isomers at benzylic position |

| | | | | |
|---|---|---|---|---|
| 210 | 105 | 68 | 150 | 2.8 |

| | |
|---|---|
| 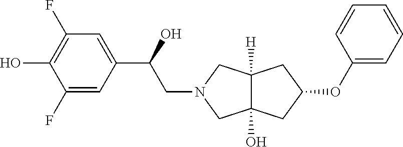 [SMILES: Oc1c(F)cc([C@@H](O)CN2C[C@H]3C[C@H](Oc4ccccc4)C[C@@]3(O)C2)cc1F] | Example 10C |

| | | | | |
|---|---|---|---|---|
| 106 | 26 | 104 | 37 | 22 |

| | |
|---|---|
| 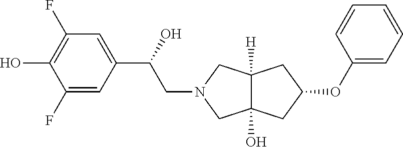 [SMILES: Oc1c(F)cc([C@H](O)CN2C[C@H]3C[C@H](Oc4ccccc4)C[C@@]3(O)C2)cc1F] | Example 10D |

| | | | | |
|---|---|---|---|---|
| 306 | 30 | 136 | 22 | 25 |

As seen in Table 2, compounds from the present disclosure have improved properties compared to comparative compounds lacking the core hydroxy group. The compounds of the present disclosure generally have lower clearance in microsomes and hepatocytes, which is believed to be associated with a more desirable pharmacokinetic profile. Furthermore, the compounds of the present disclosure have less activity in the hERG Qpatch assay, which is believed to be associated with an improved cardiosafety profile.

1. A compound of Formula I (II: 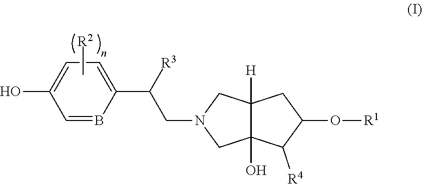
or a pharmaceutically acceptable salt thereof, wherein:
R¹ is a C₃₋₈cycloalkyl, C₃₋₇heterocyclyl, phenyl, naphthyl, or heteroaryl, each of which is optionally substituted with one or more R⁵;
R² is OH, CN, halogen, OR⁶, SH, SR⁶, C₁₋₆alkyl, haloC₁₋₆alkyl, NH₂, NHR⁶, hydroxyC₁₋₆alkyl, N(R⁶)(R^{6′}), NHS(O)₂R⁶, or NHCOR⁶;
R³ is H, O, or OH;
R⁴ is H or OH;
R⁵ is halogen, OH, C₁₋₆alkyl, haloC₁₋₆alkyl, OR⁶, CN, NH₂, NHR⁶, N(R⁶)(R^{6′}), SH, SR⁶, SOR⁶, SO₂R⁶, SO₂NHR⁶, SO₂N(R⁶)(R^{6′}), CONH₂, CONHR⁶, or CON(R⁶)(R^{6′});
each R⁶ and R^{6′} is independently selected from the group consisting of H, O—C₁₋₆alkyl, C₁₋₆alkyl, and haloC₁₋₆alkyl;
B is N or CRx;
each Rx is independently H, C₁₋₃alkyl, or halogen; and
each n is independently 0, 1, or 2.

2. A compound according to claim 1 of Formula (II): 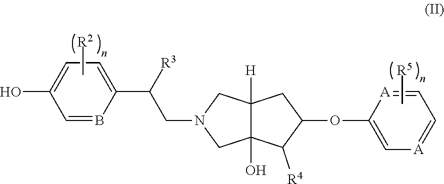
or a pharmaceutically acceptable salt thereof, wherein:
R² is OH, CN, halogen, OR⁶, SH, SR⁶, C₁₋₆ alkyl, haloC₁₋₆alkyl, NH₂, NHR⁶, hydroxyC₁₋₆ alkyl, N(R⁶)(R^{6′}), NHS(O)₂R⁶, or NHCOR⁶;
R³ is H, O, or OH;
R⁴ is H or OH;
R⁵ is halogen, OH, C₁₋₆ alkyl, haloC₁₋₆alkyl, OR⁶, CN, NH₂, NHR⁶, N(R⁶)(R^{6′}), SH, SR⁶, SOR⁶, SO₂R⁶, SO₂NHR⁶, SO₂N(R⁶)(R^{6′}), CONH₂, CONHR⁶, or CON(R⁶)(R^{6′});
each R⁶ and R^{6′} is independently selected from the group consisting of H, O—C₁₋₆ alkyl, C₁₋₆ alkyl, and haloC₁₋₆alkyl;
B is N or CRx;
each Rx is independently H, C₁₋₃ alkyl, or halogen; and
each n is independently 0, 1, or 2.

3. A compound according to claim 1 of Formula (III); 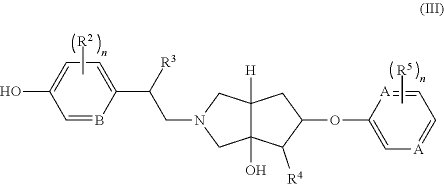
or a pharmaceutically acceptable salt thereof, wherein:
R² is halogen;
R³ is H or OH;
R⁴ is H or OH;
R⁵ is halogen;
each A is independently N or CH, provided that when one A is N the other is CH;
B is N or CH; and
each n is independently 0, 1, or 2.

4. The compound according to claim 3 of Formula (IIIa): 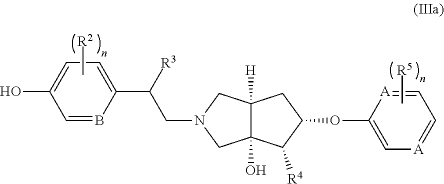
or a pharmaceutically acceptable salt thereof.

5. The compound according to claim 3 of Formula (IIIb): 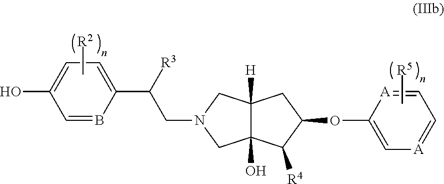
or a pharmaceutically acceptable salt thereof.

6. The compound according to claim 3 of Formula (IIIc): 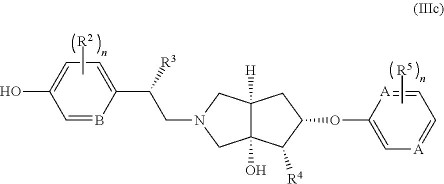
or a pharmaceutically acceptable salt thereof.

7. The compound according to claim 3 of Formula (IIId): 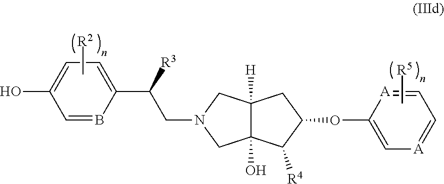
or a pharmaceutically acceptable salt thereof.

8. The compound according to claim 3 of Formula (IIIe): 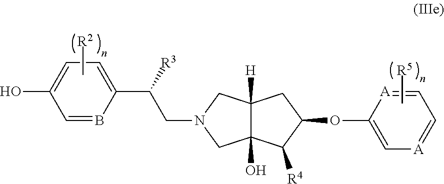
or a pharmaceutically acceptable salt thereof.

9. The compound according to claim 3 of Formula (IIIf): 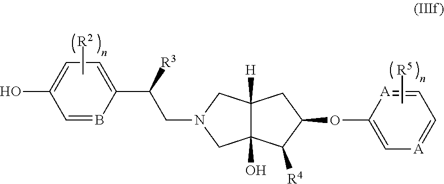
or a pharmaceutically acceptable salt thereof.

10. The compound of Formula (I) according to claim 1, wherein R⁵ is F.

11. The compound of Formula (I) according to claim 1, wherein R² is F or Cl.

12. The compound of Formula (I) according to claim 1, wherein R³ is H.

13. The compound of Formula (I) according to claim 1, wherein R³ is OH.

14. The compound of Formula (I) according to claim 1, wherein R⁴ is H.

15. The compound of Formula (I) according to claim 1, wherein R⁴ is OH.

16. The compound of Formula (I) according to claim 1, wherein B is N.

17. The compound of Formula (I) according to claim 1, wherein B is CH.

18. The compound of Formula (I) according to claim 1, wherein A is N, provided that when one A is N the other A is CH.

19. The compound of Formula (I) according to claim 1, wherein A is CH.

20. The compound of Formula (I) according to claim 1, wherein R² is halogen, C₁₋₆alkyl, haloC₁₋₆alkyl, or hydroxyC₁₋₆alkyl.

21. The compound of Formula (I) according to claim 1, wherein R² is halogen, C₁₋₆alkyl, or haloC₁₋₆alkyl.

22. The compound of Formula (I) according to claim 1, wherein R⁵ is halogen, OH, C₁₋₆alkyl, OR⁶, CN, SH, or SR⁶.

23. The compound of Formula (I) according to claim 1, wherein R⁵ is halogen, OH, C₁₋₆alkyl, or OR⁶.

24. The compound of Formula (I) according to claim 1, wherein R⁵ is halogen, OH, or C₁₋₆alkyl.

25. A compound which is:
(3aS,5S,6aR)-5-(2-fluorophenoxy)-2-((S)-2-hydroxy-2-(5-hydroxypyridin-2-yl)ethyl)hexahydrocyclopenta[c]pyrrol-3a(1H)-ol;
(3aS,5S,6aR)-5-(2-fluorophenoxy)-2-((R)-2-hydroxy-2-(5-hydroxypyridin-2-yl)ethyl)hexahydrocyclopenta[c]pyrrol-3a(1H)-ol;
(3aR,5R,6aS)-5-(2-fluorophenoxy)-2-((S)-2-hydroxy-2-(5-hydroxypyridin-2-yl)ethyl)hexahydrocyclopenta[c]pyrrol-3a(1H)-ol;
(3aR,5R,6aS)-5-(2-fluorophenoxy)-2-((R)-2-hydroxy-2-(5-hydroxypyridin-2-yl)ethyl)hexahydrocyclopenta[c]pyrrol-3a(1H)-ol;
(3aS,4S,5S,6aR)-5-(2-fluorophenoxy)-2-((S)-2-hydroxy-2-(5-hydroxypyridin-2-yl)ethyl)hexahydrocyclopenta[c]pyrrole-3a,4(1H)-diol;
(3aS,4S,5S,6aR)-5-(2-fluorophenoxy)-2-((R)-2-hydroxy-2-(5-hydroxypyridin-2-yl)ethyl)hexahydrocyclopenta[c]pyrrole-3a,4(1H)-diol;
(3aR,4R,5R,6aS)-5-(2-fluorophenoxy)-2-((S)-2-hydroxy-2-(5-hydroxypyridin-2-yl)ethyl)hexahydrocyclopenta[c]pyrrole-3a,4(1H)-diol;
(3aR,4R,5R,6aS)-5-(2-fluorophenoxy)-2-((R)-2-hydroxy-2-(5-hydroxypyridin-2-yl)ethyl)hexahydrocyclopenta[c]pyrrole-3a,4(1H)-diol;
(3aS,4S,5S,6aR)-2-((S)-2-(6-fluoro-5-hydroxypyridin-2-yl)-2-hydroxyethyl)-5-phenoxyhexahydrocyclopenta[c]pyrrole-3a,4(1H)-diol;
(3aS,4S,5S,6aR)-2-((R)-2-(6-fluoro-5-hydroxypyridin-2-yl)-2-hydroxyethyl)-5-phenoxyhexahydrocyclopenta[c]pyrrole-3a,4(1H)-diol;
(3aR,4R,5R,6aS)-2-((S)-2-(6-fluoro-5-hydroxypyridin-2-yl)-2-hydroxyethyl)-5-phenoxyhexahydrocyclopenta[c]pyrrole-3a,4(1H)-diol;
(3aR,4R,5R,6aS)-2-((R)-2-(6-fluoro-5-hydroxypyridin-2-yl)-2-hydroxyethyl)-5-phenoxyhexahydrocyclopenta[c]pyrrole-3a,4(1H)-diol;
(3aS,4S,5S,6aR)-2-((S)-2-(6-chloro-5-hydroxypyridin-2-yl)-2-hydroxyethyl)-5-phenoxyhexahydrocyclopenta[c]pyrrole-3a,4(1H)-diol;
(3aS,4S,5S,6aR)-2-((R)-2-(6-chloro-5-hydroxypyridin-2-yl)-2-hydroxyethyl)-5-phenoxyhexahydrocyclopenta[c]pyrrole-3a,4(1H)-diol;
(3aR,4R,5R,6aS)-2-((S)-2-(6-chloro-5-hydroxypyridin-2-yl)-2-hydroxyethyl)-5-phenoxyhexahydrocyclopenta[c]pyrrole-3a,4(1H)-diol;
(3aR,4R,5R,6aS)-2-((R)-2-(6-chloro-5-hydroxypyridin-2-yl)-2-hydroxyethyl)-5-phenoxyhexahydrocyclopenta[c]pyrrole-3a,4(1H)-diol;
(3aS,4S,5S,6aR)-2-((R)-2-(3,5-difluoro-4-hydroxyphenyl)-2-hydroxyethyl)-5-phenoxyhexahydrocyclopenta[c]pyrrole-3a,4(1H)-diol;
(3aS,4S,5S,6aR)-2-((S)-2-(3,5-difluoro-4-hydroxyphenyl)-2-hydroxyethyl)-5-phenoxyhexahydrocyclopenta[c]pyrrole-3a,4(1H)-diol;
(3aR,4R,5R,6aS)-2-((R)-2-(3,5-difluoro-4-hydroxyphenyl)-2-hydroxyethyl)-5-phenoxyhexahydrocyclopenta[c]pyrrole-3a,4(1H)-diol;
(3aR,4R,5R,6aS)-2-((S)-2-(3,5-difluoro-4-hydroxyphenyl)-2-hydroxyethyl)-5-phenoxyhexahydrocyclopenta[c]pyrrole-3a,4(1H)-diol;
(3aS,4S,5S,6aR)-2-((R)-2-(3,5-difluoro-4-hydroxyphenyl)-2-hydroxyethyl)-5-(2-fluorophenoxy)hexahydrocyclopenta[c]pyrrole-3a,4(1H)-diol;
(3aS,4S,5S,6aR)-2-((S)-2-(3,5-difluoro-4-hydroxyphenyl)-2-hydroxyethyl)-5-(2-fluorophenoxy)hexahydrocyclopenta[c]pyrrole-3a,4(1H)-diol;
(3aR,4R,5R,6aS)-2-((R)-2-(3,5-difluoro-4-hydroxyphenyl)-2-hydroxyethyl)-5-(2-fluorophenoxy)hexahydrocyclopenta[c]pyrrole-3a,4(1H)-diol;
(3aR,4R,5R,6aS)-2-((S)-2-(3,5-difluoro-4-hydroxyphenyl)-2-hydroxyethyl)-5-(2-fluorophenoxy)hexahydrocyclopenta[c]pyrrole-3a,4(1H)-diol;
(3aS,5S,6aR)-2-((S)-2-hydroxy-2-(5-hydroxypyridin-2-yl)ethyl)-5-phenoxyhexahydrocyclopenta[c]pyrrol-3a(1H)-ol;
(3aS,5S,6aR)-2-((R)-2-hydroxy-2-(5-hydroxypyridin-2-yl)ethyl)-5-phenoxyhexahydrocyclopenta[c]pyrrol-3a(1H)-ol;
(3aR,5R,6aS)-2-((S)-2-hydroxy-2-(5-hydroxypyridin-2-yl)ethyl)-5-phenoxyhexahydrocyclopenta[c]pyrrol-3a(1H)-ol;
(3aR,5R,6aS)-2-((S)-2-hydroxy-2-(5-hydroxypyridin-2-yl)ethyl)-5-phenoxyhexahydrocyclopenta[c]pyrrol-3a(1H)-ol;
(3aS,5S,6aR)-2-((R)-2-(3,5-difluoro-4-hydroxyphenyl)-2-hydroxyethyl)-5-(2-fluorophenoxy)hexahydrocyclopenta[c]pyrrol-3a(1H)-ol;
(3aS,5S,6aR)-2-((S)-2-(3,5-difluoro-4-hydroxyphenyl)-2-hydroxyethyl)-5-(2-fluorophenoxy)hexahydrocyclopenta[c]pyrrol-3a(1H)-ol;
(3aR,5R,6aS)-2-((R)-2-(3,5-difluoro-4-hydroxyphenyl)-2-hydroxyethyl)-5-(2-fluorophenoxy)hexahydrocyclopenta[c]pyrrol-3a(1H)-ol;
(3aR,5R,6aS)-2-((S)-2-(3,5-difluoro-4-hydroxyphenyl)-2-hydroxyethyl)-5-(2-fluorophenoxy)hexahydrocyclopenta[c]pyrrol-3a(1H)-ol;
(3aS,5S,6aR)-2-((S)-2-(6-fluoro-5-hydroxypyridin-2-yl)-2-hydroxyethyl)-5-phenoxyhexahydrocyclopenta[c]pyrrol-3a(1H)-ol;
(3aS,5S,6aR)-2-((R)-2-(6-fluoro-5-hydroxypyridin-2-yl)-2-hydroxyethyl)-5-phenoxyhexahydrocyclopenta[c]pyrrol-3a(1H)-ol;
(3aR,5R,6aS)-2-((S)-2-(6-fluoro-5-hydroxypyridin-2-yl)-2-hydroxyethyl)-5-phenoxyhexahydrocyclopenta[c]pyrrol-3a(1H)-ol;
(3aR,5R,6aS)-2-((R)-2-(6-fluoro-5-hydroxypyridin-2-yl)-2-hydroxyethyl)-5-phenoxyhexahydrocyclopenta[c]pyrrol-3a(1H)-ol;
(3aS,5S,6aR)-2-((R)-2-(3,5-difluoro-4-hydroxyphenyl)-2-hydroxyethyl)-5-phenoxyhexahydrocyclopenta[c]pyrrol-3a(1H)-ol;
(3aS,5S,6aR)-2-((S)-2-(3,5-difluoro-4-hydroxyphenyl)-2-hydroxyethyl)-5-phenoxyhexahydrocyclopenta[c]pyrrol-3a(1H)-ol;
(3aR,5R,6aS)-2-((R)-2-(3,5-difluoro-4-hydroxyphenyl)-2-hydroxyethyl)-5-phenoxyhexahydrocyclopenta[c]pyrrol-3a(1H)-ol;
(3aR,5R,6aS)-2-((S)-2-(3,5-difluoro-4-hydroxyphenyl)-2-hydroxyethyl)-5-phenoxyhexahydrocyclopenta[c]pyrrol-3a(1H)-ol;
(3aS,5S,6aR)-2-((S)-2-(6-chloro-5-hydroxypyridin-2-yl)-2-hydroxyethyl)-5-phenoxyhexahydrocyclopenta[c]pyrrol-3a(1H)-ol;
(3aS,5S,6aR)-2-((R)-2-(6-chloro-5-hydroxypyridin-2-yl)-2-hydroxyethyl)-5-phenoxyhexahydrocyclopenta[c]pyrrol-3a(1H)-ol;
(3aS,5S,6aR)-2-((R)-2-(3,5-difluoro-4-hydroxyphenyl)-2-hydroxyethyl)-5-(2,4-difluorophenoxy)hexahydrocyclopenta[c]pyrrol-3a(1H)-ol;
(3aS,5S,6aR)-2-((S)-2-(3,5-difluoro-4-hydroxyphenyl)-2-hydroxyethyl)-5-(2,4-difluorophenoxy)hexahydrocyclopenta[c]pyrrol-3a(1H)-ol;
(3aR,5R,6aS)-2-((R)-2-(3,5-difluoro-4-hydroxyphenyl)-2-hydroxyethyl)-5-(2,4-difluorophenoxy)hexahydrocyclopenta[c]pyrrol-3a(1H)-ol;
(3aR,5R,6aS)-2-((S)-2-(3,5-difluoro-4-hydroxyphenyl)-2-hydroxyethyl)-5-(2,4-difluorophenoxy)hexahydrocyclopenta[c]pyrrol-3a(1H)-ol;
(3aS,5S,6aR)-5-(2,4-difluorophenoxy)-2-((R)-2-hydroxy-2-(4-hydroxyphenyl)ethyl)hexahydrocyclopenta[c]pyrrol-3a(1H)-ol;
(3aS,5S,6aR)-5-(2,4-difluorophenoxy)-2-((S)-2-hydroxy-2-(4-hydroxyphenyl)ethyl)hexahydrocyclopenta[c]pyrrol-3a(1H)-ol;
(3aR,5R,6aS)-5-(2,4-difluorophenoxy)-2-((R)-2-hydroxy-2-(4-hydroxyphenyl)ethyl)hexahydrocyclopenta[c]pyrrol-3a(1H)-ol;
(3aR,5R,6aS)-5-(2,4-difluorophenoxy)-2-((S)-2-hydroxy-2-(4-hydroxyphenyl)ethyl)hexahydrocyclopenta[c]pyrrol-3a(1H)-ol;
(3aS,5S,6aR)-2-(3,5-difluoro-4-hydroxyphenethyl)-5-(2-fluorophenoxy)hexahydrocyclopenta[c]pyrrol-3a(1H)-ol;
(3aR,5R,6aS)-2-(3,5-difluoro-4-hydroxyphenethyl)-5-(2-fluorophenoxy)hexahydrocyclopenta[c]pyrrol-3a(1H)-ol;
(3aS,5S,6aR)-2-(3,5-difluoro-4-hydroxyphenethyl)-5-(2,4-difluorophenoxy)hexahydrocyclopenta[c]pyrrol-3a(1H)-ol;
(3aR,5R,6aS)-2-(3,5-difluoro-4-hydroxyphenethyl)-5-(2,4-difluorophenoxy)hexahydrocyclopenta[c]pyrrol-3a(1H)-ol;
(3aS,5S,6aR)-2-(3,5-difluoro-4-hydroxyphenethyl)-5-phenoxyhexahydrocyclopenta[c]pyrrol-3a(1H)-ol;
(3aR,5R,6aS)-2-(3,5-difluoro-4-hydroxyphenethyl)-5-phenoxyhexahydrocyclopenta[c]pyrrol-3a(1H)-ol;
(3aS,5S,6aR)-5-((3-fluoropyridin-2-yl)oxy)-2-(4-hydroxyphenethyl)hexahydrocyclopenta[c]pyrrol-3a(1H)-ol;
(3aR,5R,6aS)-5-((3-fluoropyridin-2-yl)oxy)-2-(4-hydroxyphenethyl)hexahydrocyclopenta[c]pyrrol-3a(1H)-ol;
(3aS,5S,6aR)-5-((2-fluoropyridin-3-yl)oxy)-2-(4-hydroxyphenethyl)hexahydrocyclopenta[c]pyrrol-3a(1H)-ol;
(3aR,5R,6aS)-5-((2-fluoropyridin-3-yl)oxy)-2-(4-hydroxyphenethyl)hexahydrocyclopenta[c]pyrrol-3a(1H)-ol;
(3aS,5S,6aR)-5-(2,4-difluorophenoxy)-2-(2-(5-hydroxypyridin-2-yl)ethyl)hexahydrocyclopenta[c]pyrrol-3a(1H)-ol;
(3aR,5R,6aS)-5-(2,4-difluorophenoxy)-2-(2-(5-hydroxypyridin-2-yl)ethyl)hexahydrocyclopenta[c]pyrrol-3a(1H)-ol;
(3aS,5S,6aR)-5-((2-fluoropyridin-3-yl)oxy)-2-(2-(5-hydroxypyridin-2-yl)ethyl)hexahydrocyclopenta[c]pyrrol-3a(1H)-ol; and
(3aR,5R,6aS)-5-((2-fluoropyridin-3-yl)oxy)-2-(2-(5-hydroxypyridin-2-yl)ethyl)hexahydrocyclopenta[c]pyrrol-3a(1H)-ol, or a pharmaceutically acceptable salt thereof.

26. A pharmaceutical composition comprising a compound according to claim 1, or a pharmaceutically acceptable salt thereof.

27. A method for the treatment of Parkinson's disease, Huntington's disease, Rett syndrome, amyotrophic lateral sclerosis, multiple sclerosis, seizure disorders, autism, autism spectrum disorders, Fragile X syndrome, tuberous sclerosis, Down's syndrome, pain, migraine, tinnitus, bipolar disorder, obsessive-compulsive disorder, anxiety disorder, post-traumatic stress disorder (PTSD), cocaine use disorder, major depressive disorder, refractory or treatment resistant depression, or suicidality comprising administering a therapeutically effective amount of a compound according to claim 1, or a pharmaceutically acceptable salt thereof to a patient in need of treatment thereof.

28. The method of claim 27, comprising administering a pharmaceutical composition comprising the compound, or a pharmaceutically acceptable salt thereof.
